(19) United States
(12) Patent Application Publication
(10) Pub. No.: US 2023/0110689 A1
(43) Pub. Date: Apr. 13, 2023

(54) AGONIST COMBINATION

(71) Applicant: Zealand Pharma A/S, Søborg (DK)

(72) Inventors: Jonathan Griffin, Søborg (DK); Per-Olof Eriksson, Søborg (DK); Wayne Russell, Søborg (DK); Mark Berner-Hansen, Søborg (DK)

(21) Appl. No.: 17/906,871

(22) PCT Filed: Mar. 29, 2021
(86) PCT No.: PCT/EP2021/058188

§ 371 Date: Sep. 21, 2022

(30) Foreign Application Priority Data
Mar. 30, 2020 (EP) 20166862.1

(51) Int. Cl. A61K 38/26; A61P 1/00

(52) U.S. Cl. CPC A61K 38/26; A61P 1/00

(57) ABSTRACT

The present invention relates to an agonist combination comprising a GLP-1 agonist and a GLP-2 agonist, for use in the treatment of a patient who has undergone surgical resection of the bowel.

### FIELD OF THE INVENTION

The present invention relates to an agonist combination comprising a GLP-1 agonist and a GLP-2 agonist, for use in the treatment of a patient who has undergone surgical resection of the bowel.

### BACKGROUND OF THE INVENTION

Bowel resections are performed to treat and prevent diseases and conditions that affect the bowel. There are various clinical situations in which a bowel resection may be advisable, for example to treat cancer in the small intestine, colon, rectum or anus, to treat or relieve symptoms of cancer that has spread to the intestine, to remove a blockage in the intestine (called a bowel obstruction), to remove as much cancer as possible (called debulking), to remove precancerous conditions before they become cancer (called prophylactic surgery), to remove parts of the colon that are damaged by an inflammatory bowel disease or diverticulitis (for example Crohns disease), or to fix a tear or hole in the intestine (called a bowel perforation).

For example, colorectal cancer was the fourth most commonly diagnosed cancer in the United States in 2013. According to the 2010-2012 National Cancer Institute cancer fact sheet, approximately 4.5% of the US population will be diagnosed with colorectal cancer at some point during their lifetime. Surgery is the most common treatment for resectable colorectal cancer.

However, whilst bowel resection is needed in order to treat and prevent bowel-related diseases, the resection itself may result in malabsorption of nutrients in the patient, which may lead to secondary complications.

There is a need in the art for therapeutic options for patients who have undergone surgical resection of the bowel which can facilitate improved recovery and adaptation of the bowel.

### SUMMARY OF THE INVENTION

The present inventors have surprisingly found that administering a combination of a GLP-1 agonist and a GLP-2 agonist, in particular a GLP-1/GLP-2 dual agonist, to a patient soon after bowel resection leads to improved recovery and adaptation of the bowel.

Broadly, the present invention relates to compounds which have agonist activity at the GLP-1 (glucagon-like peptide 1) and/or GLP-2 (glucagon-like peptide 2) receptors for use in the treatment of patients who have undergone bowel resection.

In one aspect the invention provides an agonist combination for use in the treatment of a patient who has undergone surgical resection of the bowel, wherein said agonist combination is administered to said patient within about 7 days of said surgical resection, and wherein said agonist combination comprises a GLP-1 agonist and a GLP-2 agonist.

The GLP-1 agonist and said GLP-2 agonist may be administered in the same or different compositions.

In one aspect the agonist combination is a dual GLP-1/GLP-2 agonist.

In one aspect the invention provides a composition comprising a GLP-1/GLP-2 dual agonist for use in the treatment of a patient who has undergone surgical resection of the bowel, wherein said composition is administered to said patient within 7 days of said surgical resection.

In one aspect the agonist combination is administered to said patient within about 6, 5, 4, 3, 2 or 1 day of said surgical resection.

In one aspect the agonist combination is administered to said patient within about 48, 44, 40, 36, 32, 28, 24, 20, 16, 12, 8 or 4 hours of said surgical resection, preferably within 24 hours of said resection.

In one aspect said patient has short bowel syndrome.

In one aspect the GLP-1 agonist, GLP-2 agonist, and/or dual agonist, is a peptide.

The GLP-2 agonist may be selected from native GLP-2, a GLP-2 analog, such as Teduglutide (WO1997039031, Shire NPS Pharmaceuticals, Takeda), Glepaglutide (WO2006117565, Zealand Pharma A/S), BC-GLP-2 (WO2019086559, Adocia), HM15912 (WO2019066586, Hanmi Pharmaceuticals), NB1002 (Naia Pharmaceuticals) or Apraglutide (WO2011050174, Vectivbio), a synthetic GLP-2 and a GLP-2 peptibody such as e.g. SHP681 (WO2019040399, Shire NPS Pharmaceuticals, Takeda).

The GLP-1 agonist may be selected from native GLP-1, a GLP-1 analog, such as Liraglutide (EP1687019, Novo Nordisk A/S), Lixisenatide, Dulaglutide, Semaglutide or Exenatide (Byetta/Bydureon, AstraZeneca/Amylin Pharmaceuticals Inc), and a synthetic GLP-1. In one aspect the GLP-1 agonist may be selected from Bydureon (Exenatide) AstraZeneca—taken once weekly, Byetta (Exenatide) AstraZeneca—taken twice daily, Lyxumia (lixisenatideLixisenatide) Sanofi—taken once daily, Trulicity (Dulaglutide) Eli Lilly—taken once weekly, Victoza (Liraglutide) Novo Nordisk—taken once daily, and Rybelsus (Semaglutide) Novo Nordisk—taken once weekly.

In one aspect of the invention, the combination of a GLP-1 agonist and a GLP-2 agonist comprises a combination of Teduglutide and Exenatide Teduglutide and Liraglutide, Glepaglutide and Semaglutide, or a combination of Glepaglutide and Exenatide.

As described herein, in one aspect of the invention the agonist combination is a GLP-1/GLP-2 dual agonist. In a further aspect the GLP-1/GLP-2 dual agonist is a compound represented by the formula:

R¹—X*-U-R²

wherein:

R¹ is hydrogen (Hy), C₁₋₄ alkyl (e.g. methyl), acetyl, formyl, benzoyl or trifluoroacetyl;

R² is NH₂ or OH;

X* is a peptide of formula I:

(I)
H-X2-EG-X5-F-X7-X8-E-X10-X11-TIL-X15-X16-X17-A-
X19-X20-X21-FI-X24-WL-X27-X28-X29-KIT-X33

wherein:

X2 is Aib or G

X5 is T or S;

X7 is T or S;

X8 is S, E or D;

X10 is L, M, V or Ψ;

X11 is A, N or S;

X15 is D or E;

X16 is G, E, A or Ψ;

X17 is Q, E, K, L or Ψ;

X19 is A, V or S;

X20 is R, K or Ψ;

X21 is D, L or E;

X24 is A, N or S;

X27 is I, Q, K, H or Y;

X28 is Q, E, A, H, Y, L, K, R or S;

X29 is H, Y, K or Q;

X33 is D or E;

U is absent or a sequence of 1-15 residues each independently selected from K, k, E, A, T, I, L and Ψ);

the molecule contains one and only one Ψ, wherein Ψ is a residue of K, k, R, Orn, Dap or Dab in which the side chain is conjugated to a substituent having the formula Z¹— or Z¹—Z²—, wherein

Z¹— is CH₃—(CH₂)₁₀₋₂₂—(CO)— or HOOC—(CH₂)₁₀₋₂₂—(CO)—; and

—Z²— is selected from —Z^{S1}—, —Z^{S1}—Z^{S2}—, —Z^{S2}—Z^{S1}, —Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, —Z^{S2}Z^{S3}Z^{S2}— wherein

Z^{S1} is isoGlu, β-Ala, isoLys, or 4-aminobutanoyl;

Z^{S2} is -(Peg3)ₘ- where m is 1, 2, or 3; and

—Z^{s3}— is a peptide sequence of 1-6 amino acid units independently selected from the group consisting of A, L, S, T, Y, Q, D, E, K, k, R, H, F and G;

and wherein at least one of X5 and X7 is T;

or a pharmaceutically acceptable salt or solvate thereof.

The various amino acid positions in peptide X* of the formulae provided here are numbered according to their linear position from N- to C-terminus in the amino acid chain.

In the present context, β-Ala and 3-Aminopropanoyl are used interchangeably.

Dual agonists having aspartic acid (Asp, D) at position 3 and glycine (Gly) in position 4 can be very potent agonists at the GLP-1 and GLP-2 receptors. However, this combination of substitutions results in compounds which are unstable and may not be suitable for long term storage in aqueous solution. Without wishing to be bound by theory, it is believed that the Asp at position 3 may isomerise to iso-Asp via a cyclic intermediate formed between the carboxylic acid functional group of its side chain and the backbone nitrogen atom of the residue at position 4.

It has now been found that molecules having glutamic acid (Glu, E) at position 3 instead of Asp are much less susceptible to such reactions and hence may be considerably more stable when stored in aqueous solution. However, replacement of Asp with Glu at position 3 in molecules having a lipophilic substituent in the middle portion of the peptide (e.g. at or near to positions 16 and 17) tends to reduce the potency at one or both of the GLP-2 receptor and the GLP-1 receptor, even though Glu is present at position 3 of the native GLP-1 molecule. Simultaneously incorporating a Thr residue at one or both of positions 5 and 7 appears to compensate for some or all of the lost potency. It is believed that further improvements in potency are also provided by incorporation of His (H), Tyr (Y), Lys (K) or Gln (Q) at position 29 instead of the Gly (G) and Thr (T) residues present in wild type human GLP-1 and 2 respectively.

In some embodiments of formula I:

X2 is Aib or G

X5 is T or S;

X7 is T or S;

X8 is S;

X10 is L or Ψ;

X11 is A or S;

X15 is D or E;

X16 is G, E, A or Ψ;

X17 is Q, E, K, L or Ψ;

X19 is A or S;

X20 is R or Ψ;

X21 is D, L or E;

X24 is A;

X27 is I, Q, K, or Y;

X28 is Q, E, A, H, Y, L, K, R or S;

X29 is H, Y or Q; and

X33 is D or E.

Where Ψ is not at X16 or X17, it may be desirable that X16 is E and X17 is Q.

In some embodiments, X11 is A and X15 is D. In other embodiments, X11 is S and X15 is E. In further embodiments, X11 is A and X15 is E.

In some embodiments, X27 is I.

In some embodiments, X29 is H. In certain of these embodiments, X28 is A and X29 is H, or X28 is E and X29 is H.

In some embodiments, X29 is Q and optionally X27 is Q.

In some embodiments, the residues at X27-X29 have a sequence selected from:

IQH;

IEH

IAH;

IHH;

IYH;

ILH;

IKH;

IRH;

ISH;

QQH;

YQH;

KQH;

IQQ;

IQY;

IQT; and

IAY.

In some embodiments, X* is a peptide of formula II:

(II)
H-X2-EG-X5-F-X7-SELATILD-X16-X17-AAR-X21-FIAWLI-
X28-X29-KITD

wherein:

X2 is Aib or G

X5 is T or S;

X7 is T or S;

X16 is G or Ψ;

X17 is Q, E, K, L or Ψ;

X21 is D or L;

X28 is Q, E, A, H, Y, L, K, R or S;

X29 is H, Y or Q;

In some embodiments of Formula I or Formula II, X16 is Ψ and X17 is Q, E, K or L. For example, X17 may be Q, or X17 may be selected from E, K and L. In other embodiments, X16 is G and X17 is Ψ.

It may be desirable that X21 is D.

X28 may be selected from Q, E and A, e.g. it may be Q or E. In some residue combinations, Q may be preferred. In others, E may be preferred, including but not limited to when X16 is G and X17 is Ψ. Alternatively, X28 may be selected from A, H, Y, L, K, R and S.

X* may be a peptide of formula III:

(III)
H[Aib]EG-X5-F-X7-SE-X10-ATILD-X16-X17-AA-X20-X21-
FIAWLI-X28-X29-KITD

wherein:

X5 is T or S;

X7 is T or S;

X10 is L or Ψ;

X16 is G, E, A or Ψ;

X17 is Q, E, K, L or Ψ;

X20 is R or Ψ;

X21 is D or L;

X28 is E, A or Q;

X29 is H, Y or Q;

and at least one of X5 and X7 is T.

X* may be a peptide of formula IV:

(IV)
H[Aib]EG-X5-F-X7-SELATILD-X16-X17-AAR-X21-FIAWLI-
X28-X29-KITD

wherein:

X5 is T or S;

X7 is T or S;

X16 is G or Ψ;

X17 is E, K, L or Ψ;

X21 is D or L;

X28 is E or A;

X29 is H, Y or Q;

and at least one of X5 and X7 is T.

In some embodiments of any of formulae I to IV, X16 is Ψ and X17 is E, K or L.

In other embodiments of formula I to IV, X16 is G and X17 is Ψ.

In either case, the following combinations of residues may also be included:

X21 is D and X28 is E;

X21 is D and X28 is A;

X21 is Land X28 is E;

X21 is Land X28 is A.

X* may be a peptide of formula V:

(V)
H[Aib]EG-X5-F-X7-SELATILD-Ψ-QAARDFIAWLI-X28-X29-
KITD

wherein

X5 is T or S;

X7 is T or S;

X28 is Q, E, A, H, Y, L, K, R or S, e.g. Q, E, A, H, Y or L;

X29 is H, Y or Q;

and at least one of X5 and X7 is T.

In some embodiments of formula III, X28 is Q or E. In some embodiments of formula III, X28 is Q. In other embodiments, X28 is A, H, Y, L, K, R or S, e.g. A, H, Y or L.

In any of the formulae or embodiments described above, the dual agonist contains one of the following combinations of residues:

X5 is S and X7 is T;

X5 is T and X7 is S;

X5 is T and X7 is T.

It may be preferred that X5 is S and X7 is T, or X5 is T and X7 is T.

In any of the formulae or embodiments described above, it may be desirable that X29 is H.

In some embodiments, Ψ is a Lys residue whose side chain is conjugated to the substituent Z¹— or Z¹—Z²—.

In some embodiments, Z¹—, alone or in combination with —Z²—, is dodecanoyl, tetradecanoyl, hexadecanoyl, octadecanoyl or eicosanoyl.

In some embodiments, Z¹—, alone or in combination with —Z²—, is:

13-carboxytridecanoyl, i.e. HOOC—(CH₂)₁₂—(CO)—;

15-carboxypentadecanoyl, i.e. HOOC—(CH₂)₁₄—(CO)—;

17-carboxyheptadecanoyl, i.e. HOOC—(CH₂)₁₆—(CO)—;

19-carboxynonadecanoyl, i.e. HOOC—(CH₂)₁₈—(CO)—; or

21-carboxyheneicosanoyl, i.e. HOOC—(CH₂)₂₀—(CO)—.

In some embodiments Z² is absent.

In some embodiments, Z² comprises Z^{S1} alone or in combination with Z^{S2} and/or Z^{S3}.

In such embodiments:

—Z^{S1}— is isoGlu, β-Ala, isoLys, or 4-aminobutanoyl;

—Z^{S2}—, when present, is -(Peg3)ₘ- where m is 1, 2, or 3; and

—Z^{S3}— is a peptide sequence of 1-6 amino acid units independently selected from the group consisting of A, L, S, T, Y, Q, D, E, K, k, R, H, F and G, such as the peptide sequence KEK.

Z² may have the formula —Z^{S1}—Z^{S3}—Z^{S2}—, where Z^{S1} is bonded to Z¹ and Z^{S2} is bonded to the side chain of the amino acid component of Ψ.

Thus, in some embodiments, —Z²— is:

isoGlu(Peg 3)₀₋₃;

β-Ala(Peg 3)₀₋₃;

isoLys(Peg3)₀₋₃; or

4-aminobutanoyl(Peg3)₀₋₃.

In further embodiments, —Z²— is:

isoGlu-KEK-(Peg3)₀₋₃.

Specific examples of the substituent Z¹—Z²— are set out below. In some embodiments, Z¹—Z²— is [17-carboxy-heptadecanoyl]-isoGlu. For example, Ψ may be K([17-carboxy-heptadecanoyl]-isoGlu). In some embodiments, Z¹—Z²— is:

[17-Carboxy-heptadecanoyl]-isoGlu-KEK-Peg3-;

[17-carboxy-heptadecanoyl]-isoGlu-Peg3-;

[19-Carboxy-nonadecanoyl]-isoGlu-;

[19-Carboxy-nonadecanoyl]-isoGlu-KEK-;

[19-Carboxy-nonadecanoyl]-isoGlu-KEK-Peg3-;

[19-carboxy-nonadecanoyl]-isoGlu-KEK-Peg3-Peg3-;

[19-carboxy-nonadecanoyl]-isoGlu-Peg3-Peg3-;

[19-carboxy-nonadecanoyl]-isoLys-Peg3-Peg3-Peg3-;

[Hexadecanoyl]-βAla-;

[Hexadecanoyl]-isoGlu-; or

Octadecanoyl-.

For example, Ψ may be:

K([17-Carboxy-heptadecanoyl]-isoGlu-KEK-Peg3);

K([17-carboxy-heptadecanoyl]-isoGlu-Peg3);

K([19-Carboxy-nonadecanoyl]-isoGlu);

K([19-Carboxy-nonadecanoyl]-isoGlu-KEK);

K([19-Carboxy-nonadecanoyl]-isoGlu-KEK-Peg3);

K([19-carboxy-nonadecanoyl]-isoGlu-KEK-Peg3-Peg3);

K([19-carboxy-nonadecanoyl]-isoGlu-Peg3-Peg3);

K([19-carboxy-nonadecanoyl]-isoLys-Peg3-Peg3-Peg3);

K([Hexadecanoyl]-βAla-;

K([Hexadecanoyl]-isoGlu); or

K(Octadecanoyl).

When present, U represents a peptide sequence of 1-15 residues each independently selected from K (i.e. L-lysine), k (i.e. D-lysine) E (Glu), A (Ala), T (Thr), I (Ile), L (Leu) and Ψ. For example, U may be 1-10 amino acids in length, 1-7 amino acids in length, 3-7 amino acids in length, 1-6 amino acids in length, or 3-6 amino acids in length.

Typically U includes at least one charged amino acid (K, k or E) and preferably two or more charged amino acids. In some embodiments it includes at least 2 positively charged amino acids (K or k), or at least 1 positively charged amino acid (K or k) and at least one negatively charged amino acid (E). In some embodiments, all amino acid residues of U (except for Ψ, if present) are charged. For example, U may be a chain of alternately positively and negatively charged amino acids.

In certain embodiments, U comprises residues selected only from K, k, E and Ψ.

In certain embodiments, U comprises residues selected only from K, k, and Ψ.

When U comprises only lysine residues (whether K or k), all residues may have an L-configuration or all may have a D-configuration. Examples include K₁₋₁₅, K₁₋₁₀ and K₁₋₇, e.g., K₃, K₄, K₅, K₆, K₇, especially K₅ and K₆. Further examples include K₁₋₁₅, K₁₋₁₀ and K₁₋₇, e.g. k₃, k₄, k₅, k₆ and k₇, especially k₅ and k₆.

Further examples of peptide sequences U include KEK, EKEKEK, EkEkEk, AKAAEK, AKEKEK and ATILEK.

In any case, one of those residues may be exchanged for Ψ. Where the sequence U contains a residue Ψ, it may be desirable that the C-terminal residue of U is Ψ. Thus, further examples of sequences U include K₁₋₁₄-Ψ, K₁₋₉-Ψ and K₁₋₆-Ψ, e.g., K₂-Ψ, K₃-Ψ, K₄-Ψ, K₅-Ψ and K₆-Ψ, especially K₄-Ψ and K₅-Ψ. Yet further examples include K₁₋₁₄-Ψ, K₁₋₉-Ψ and K₁₋₆-Ψ, e.g. K₂-Ψ, K₃-Ψ, K₄-Ψ, K₅-Ψ and K₆-Ψ, especially K₄-Ψ and K₅-Ψ. Yet further examples include KELΨ, EKEKEΨ, EkEkEΨ AKAAEΨ, AKEKEΨ and ATILEΨ.

In some embodiments, U is absent.

In some embodiments, R¹ is Hy and/or R² is OH.

The peptide X* or the peptide X*-U may have the sequence:

H[Aib]EGTFSSELATILDΨEAARDFIAWLIEHKITD;
H[Aib]EGSFTSELATILDΨEAARDFIAWLIEHKITD;
H[Aib]EGTFTSELATILDΨEAARDFIAWLIEHKITD;
H[Aib]EGTFSSELATILDΨKAARDFIAWLIEHKITD;
H[Aib]EGSFTSELATILDΨKAARDFIAWLIEHKITD;
H[Aib]EGTFTSELATILDΨKAARDFIAWLIEHKITD;
H[Aib]EGTFSSELATILDΨWAARDFIAWLIEHKITD;
H[Aib]EGSFTSELATILDΨWAARDFIAWLIEHKITD;
H[Aib]EGTFTSELATILDΨWAARDFIAWLIEHKITD;
H[Aib]EGTFSSELATILDΨLAARDFIAWLIEHKITD;
H[Aib]EGSFTSELATILDΨLAARDFIAWLIEHKITD;
H[Aib]EGTFTSELATILDΨLAARDFIAWLIEHKITD;
H[Aib]EGTFSSELATILDΨLAARDFIAWLIAHKITD;
H[Aib]EGSFTSELATILDΨLAARDFIAWLIAHKITD;
H[Aib]EGTFTSELATILDΨLAARDFIAWLIAHKITD;
H[Aib]EGTFTSELATILDΨEAARLFIAWLIEHKITD;
H[Aib]EGTFSSELATILDΨQAARDFIAWLIQHKITD;
H[Aib]EGSFTSELATILDΨQAARDFIAWLIQHKITD;
H[Aib]EGTFTSELATILDΨQAARDFIAWLIQHKITD;
H[Aib]EGTFSSELATILDΨQAARDFIAWLIEHKITD;
H[Aib]EGTFSSELATILDΨQAARDFIAWLIAHKITD;
H[Aib]EGSFTSELATILDΨQAARDFIAWLIAHKITD;
H[Aib]EGTFTSELATILDΨQAARDFIAWLIAHKITD;
H[Aib]EGSFTSELATILDΨQAARDFIAWLIEHKITD;
H[Aib]EGTFTSELATILDΨQAARDFIAWLIEHKITD;
H[Aib]EGSFTSELATILDΨQAARDFIAWLIHHKITD;
H[Aib]EGSFTSELATILDΨQAARDFIAWLIYHKITD;
H[Aib]EGSFTSELATILDΨQAARDFIAWLILHKITD;
H[Aib]EGSFTSELATILDΨQAARDFIAWLIKHKITD;
H[Aib]EGSFTSELATILDΨQAARDFIAWLIRHKITD;
H[Aib]EGSFTSELATILDΨQAARDFIAWLISHKITD
H[Aib]EGSFTSELATILDΨQAARDFIAWLQQHKITD;
H[Aib]EGSFTSELATILDΨQAARDFIAWLYQHKITD;
H[Aib]EGSFTSELATILDΨQAARDFIAWLKQHKITD;
H[Aib]EGSFTSELATILDΨQAARDFIAWLIQQKITD;
H[Aib]EGSFTSELATILDΨQAARDFIAWLIQYKITD;
H[Aib]EGTFSSELSTILEΨQASREFIAWLIAYKITE;
H[Aib]EGTFSSELATILDEQAARDFIAWLIAHKITDkkkkkΨ;
H[Aib]EGTFTSELATILDEQAARDFIAWLIAHKITDkkkkkΨ;
H[Aib]EGSFTSELATILDEQAARDFIAWLIEHKITDkkkkkΨ;
H[Aib]EGSFTSEΨATILDEQAARDFIAWLIEHKITD;
H[Aib]EGSFTSELATILEGΨAARDFIAWLIEHKITD;
H[Aib]EGSFTSELATILDEQAAΨDFIAWLIEHKITD;
H[Aib]EGTFTSELATILDEQAAΨDFIAWLIEHKITD;
H[Aib]EGTFTSEΨATILDEQAARDFIAWLIEHKITD;
H[Aib]EGSFTSELATILDAΨAARDFIAWLIEHKITD;
or
H[Aib]EGSFTSELATILDAKAAΨDFIAWLIEHKITD.

The peptide X* or the peptide X*-U may have the sequence:

H[Aib]EGTFSSELATILD[K*]EAARDFIAWLIEHKITD;
H[Aib]EGSFTSELATILD[K*]EAARDFIAWLIEHKITD;
H[Aib]EGTFTSELATILD[K*]EAARDFIAWLIEHKITD;
H[Aib]EGTFSSELATILD[K*]KAARDFIAWLIEHKITD;
H[Aib]EGSFTSELATILD[K*]KAARDFIAWLIEHKITD;
H[Aib]EGTFTSELATILD[K*]KAARDFIAWLIEHKITD;
H[Aib]EGTFSSELATILDG[K*]AARDFIAWLIEHKITD;
H[Aib]EGSFTSELATILDG[K*]AARDFIAWLIEHKITD;
H[Aib]EGTFTSELATILDG[K*]AARDFIAWLIEHKITD;
H[Aib]EGTFSSELATILD[K*]LAARDFIAWLIEHKITD;
H[Aib]EGSFTSELATILD[K*]LAARDFIAWLIEHKITD;
H[Aib]EGTFTSELATILD[K*]LAARDFIAWLIEHKITD;
H[Aib]EGTFSSELATILD[K*]LAARDFIAWLIAHKITD;
H[Aib]EGSFTSELATILD[K*]LAARDFIAWLIAHKITD;
H[Aib]EGTFTSELATILD[K*]LAARDFIAWLIAHKITD;
H[Aib]EGTFTSELATILD[K*]EAARLFIAWLIEHKITD;
H[Aib]EGTFSSELATILD[K*]QAARDFIAWLIQHKITD;
H[Aib]EGSFTSELATILD[K*]QAARDFIAWLIQHKITD;
H[Aib]EGTFTSELATILD[K*]QAARDFIAWLIQHKITD;
H[Aib]EGTFSSELATILD[K*]QAARDFIAWLIEHKITD;
H[Aib]EGTFSSELATILD[K*]QAARDFIAWLIAHKITD;
H[Aib]EGSFTSELATILD[K*]QAARDFIAWLIAHKITD;
H[Aib]EGTFTSELATILD[K*]QAARDFIAWLIAHKITD;
H[Aib]EGSFTSELATILD[K*]QAARDFIAWLIEHKITD;
H[Aib]EGTFTSELATILD[K*]QAARDFIAWLIEHKITD;
H[Aib]EGSFTSELATILD[K*]QAARDFIAWLIHHKITD;
H[Aib]EGSFTSELATILD[K*]QAARDFIAWLIYHKITD;
H[Aib]EGSFTSELATILD[K*]QAARDFIAWLILHKITD;
H[Aib]EGSFTSELATILD[K*]QAARDFIAWLIKHKITD;
H[Aib]EGSFTSELATILD[K*]QAARDFIAWLIRHKITD;
H[Aib]EGSFTSELATILD[K*]QAARDFIAWLISHKITD;
H[Aib]EGSFTSELATILD[K*]QAARDFIAWLQQHKITD;
H[Aib]EGSFTSELATILD[K*]QAARDFIAWLYQHKITD;
H[Aib]EGSFTSELATILD[K*]QAARDFIAWLKQHKITD;
H[Aib]EGSFTSELATILD[K*]QAARDFIAWLIQQKITD;
H[Aib]EGSFTSELATILD[K*]QAARDFIAWLIQYKITD;
H[Aib]EGTFSSELSTILE[K*]QASREFIAWLIAYKITE;
H[Aib]EGTFSSELATILDEQAARDFIAWLIAHKITDkkkkk[k*]
H[Aib]EGTFTSELATILDEQAARDFIAWLIAHKITDkkkkk[k*]
H[AIb]EGSFTSELATILDEQAARDFIAWLIEHKITDkkkkk[k*]
H[Aib]EGSFTSE[K*]ATILDEQAARDFIAWLIEHKITD;
H[AIb]EGSFTSELATILEG[K*]AARDFIAWLIEHKITD;
H[Aib]EGSFTSELATILDEQAA[K*]DFIAWLIEHKITD;
H[Aib]EGTFTSELATILDEQAA[K*]DFIAWLIEHKITD;
H[Aib]EGTFTSE[K*]ATILDEQAARDFIAWLIEHKITD;
H[Aib]EGSFTSELATILDA[K*]AARDFIAWLIEHKITD;
or
H[Aib]EGSFTSELATILDAKAA[K*]DFIAWLIEHKITD;

wherein K* or k* indicates an L or D lysine residue respectively in which the side chain is conjugated to the substituent Z¹— or Z¹Z²—.

For example, the peptide X* or the peptide X*-U may have the sequence:

H[Aib]EGTFSSELATILD[K([17-carboxy-heptadecanoyl]-isoGlu)]EAARDFIAWLIEHKITD;
H[Aib]EGSFTSELATILD[K([17-carboxy-heptadecanoyl]-isoGlu)]EAARDFIAWLIEHKITD;
H[Aib]EGTFTSELATILD[K([17-carboxy-heptadecanoyl]-isoGlu)]EAARDFIAWLIEHKITD;
H[Aib]EGTFSSELATILD[K([17-carboxy-heptadecanoyl]-isoGlu)]KAARDFIAWLIEHKITD;
H[Aib]EGSFTSELATILD[K([17-carboxy-heptadecanoyl]-isoGlu)]KAARDFIAWLIEHKITD;
H[Aib]EGTFTSELATILD[K([17-carboxy-heptadecanoyl]-isoGlu)]KAARDFIAWLIEHKITD;
H[Aib]EGTFSSELATILDG[K([17-carboxy-heptadecanoyl]-isoGlu)]AARDFIAWLIEHKITD;
H[Aib]EGSFTSELATILDG[K([17-carboxy-heptadecanoyl]-isoGlu)]AARDFIAWLIEHKITD;
H[Aib]EGTFTSELATILDG[K([17-carboxy-heptadecanoyl]-isoGlu)]AARDFIAWLIEHKITD;
H[Aib]EGTFSSELATILD[K([17-carboxy-heptadecanoyl]-isoGlu)]LAARDFIAWLIEHKITD;
H[Aib]EGSFTSELATILD[K([17-carboxy-heptadecanoyl]-isoGlu)]LAARDFIAWLIEHKITD;
H[Aib]EGTFTSELATILD[K([17-carboxy-heptadecanoyl]-isoGlu)]LAARDFIAWLIEHKITD;
H[Aib]EGTFSSELATILD[K([17-carboxy-heptadecanoyl]-isoGlu)]LAARDFIAWLIAHKITD;
H[Aib]EGSFTSELATILD[K([17-carboxy-heptadecanoyl]-isoGlu)]LAARDFIAWLIAHKITD;
H[Aib]EGTFTSELATILD[K([17-carboxy-heptadecanoyl]-isoGlu)]LAARDFIAWLIAHKITD;
H[Aib]EGTFTSELATILD[K([17-carboxy-heptadecanoyl]-isoGlu)]EAARLFIAWLIEHKITD;
H[Aib]EGTFSSELATILD[K([17-carboxy-heptadecanoyl]-isoGlu)]QAARDFIAWLIQHKITD;
H[Aib]EGSFTSELATILD[K([17-carboxy-heptadecanoyl]-isoGlu)]QAARDFIAWLIQHKITD;
H[Aib]EGTFTSELATILD[K([17-carboxy-heptadecanoyl]-isoGlu)]QAARDFIAWLIQHKITD;
H[Aib]EGTFSSELATILD[K([17-carboxy-heptadecanoyl]-isoGlu)]QAARDFIAWLIEHKITD;
H[Aib]EGTFSSELATILD[K([17-carboxy-heptadecanoyl]-isoGlu)]QAARDFIAWLIAHKITD;
H[Aib]EGSFTSELATILD[K([17-carboxy-heptadecanoyl]-isoGlu)]QAARDFIAWLIAHKITD;
H[Aib]EGTFTSELATILD[K([17-carboxy-heptadecanoyl]-isoGlu)]QAARDFIAWLIAHKITD;
H[Aib]EGSFTSELATILD[K([17-carboxy-heptadecanoyl]-isoGlu)]QAARDFIAWLIEHKITD;
H[Aib]EGTFTSELATILD[K([17-carboxy-heptadecanoyl]-isoGlu)]QAARDFIAWLIEHKITD;
H[Aib]EGSFTSELATILD[K([17-carboxy-heptadecanoyl]-isoGlu)]QAARDFIAWLIHHKITD;
H[Aib]EGSFTSELATILD[K([17-carboxy-heptadecanoyl]-isoGlu)]QAARDFIAWLIYHKITD;
H[Aib]EGSFTSELATILD[K([17-carboxy-heptadecanoyl]-isoGlu)]QAARDFIAWLILHKITD;
H[Aib]EGSFTSELATILD[K([17-carboxy-heptadecanoyl]-isoGlu)]QAARDFIAWLIKHKITD;
H[Aib]EGSFTSELATILD[K([17-carboxy-heptadecanoyl]-isoGlu)]QAARDFIAWLIRHKITD;
H[Aib]EGSFTSELATILD[K([17-carboxy-heptadecanoyl]-isoGlu)]QAARDFIAWLISHKITD;
H[Aib]EGSFTSELATILD[K([Hexadecanoyl]-βAla)]QAARDFIAWLQQHKITD;
H[Aib]EGSFTSELATILD[K([17-carboxy-heptadecanoyl]iso-Glu-
Peg3)]QAARDFIAWLYQHKITD;
H[Aib]EGSFTSELATILD[K([19-carboxy-nonadecanoyl]iso-Glu-Peg3-
Peg3)]QAARDFIAWLKQHKITD;
H[Aib]EGSFTSELATILD[K([19-carboxy-nonadecanoyl]iso-Lys-Peg3-Peg3-
Peg3)]QAARDFIAWLIQQKITD;
H[Aib]EGSFTSELATILD[K(Octadecanoyl)]QAARDFIAWLIQYKITD;
H[Aib]EGTFSSELSTILE[K(Hexadecanoyl-isoGlu)]QASREFIAWLIAYKITE;
H[Aib]EGTFSSELATILDEQAARDFIAWLIAHKITDkkkkkk([17-carboxy-Heptadecanoyl]-
isoGlu)];
H[Aib]EGTFTSELATILDEQAARDFIAWLIAHKITDkkkkkk([17-carboxy-Heptadecanoyl]-
isoGlu)];
H[Aib]EGSFTSELATILDEQAARDFIAWLIEHKITDkkkkkk([17-carboxy-Heptadecanoyl]-
isoGlu)];
H[Aib]EGTFTSELATILD[K([19-Carboxy-nonadecanoyl]-isoGlu)]QAARDFIAWLIQHKITD;
H[Aib]EGSFTSE[K([19-carboxy-nonadecanoyl]iso-Glu-Peg3-
Peg3)]ATILDEQAARDFIAWLIEHKITD;
H[Aib]EGSFTSELATILD[K([19-carboxy-nonadecanoyl]iso-Glu-Peg3-
Peg3)]KAARDFIAWLIEHKITD;
H[Aib]EGSFTSELATILEG[K([19-carboxy-nonadecanoyl]iso-Glu-Peg3-
Peg3)]AARDFIAWLIEHKITD;
H[Aib]EGSFTSELATILDEQAA[K([19-carboxy-nonadecanoyl]iso-Glu-Peg3-
Peg3)]DFIAWLIEHKITD;
H[Aib]EGTFTSELATILDEQAA[K([19-carboxy-nonadecanoyl]iso-Glu-Peg3-
Peg3)]DFIAWLIEHKITD;
H[Aib]EGTFSSELATILD[K([17-Carboxy-heptadecanoyl]-isoGlu-KEK-
Peg3)]QAARDFIAWLIQHKITD;
H[Aib]EGTFSSELATILD[K([19-Carboxy-nonadecanoyl]-isoGlu-KEK-
Peg3)]QAARDFIAWLIQHKITD;
H[Aib]EGTFSSELATILD[K([17-Carboxy-heptadecanoyl]-isoGlu-KEK-
Peg3)]QAARDFIAWLIEHKITD;
H[Aib]EGTFSSELATILD[K([19-Carboxy-nonadecanoyl]-isoGlu-KEK-
Peg3)]QAARDFIAWLIEHKITD;
H[Aib]EGTFTSELATILD[K([19-Carboxy-nonadecanoyl]-isoGlu-
KEK)]QAARDFIAWLIQHKITD;
H[Aib]EGTFTSELATILD[K([19-Carboxy-nonadecanoyl]-isoGlu-KEK-
Peg3)]QAARDFIAWLIQHKITD;
H[Aib]EGSFTSE[K([19-Carboxy-nonadecanoyl]-isoGlu-KEK-
Peg3)]ATILDEQAARDFIAWLIEHKITD;
H[Aib]EGTFTSE[K([19-Carboxy-nonadecanoyl]-isoGlu-KEK-
Peg3)]ATILDEQAARDFIAWLIEHKITD;
H[Aib]EGSFTSE[K([19-carboxy-nonadecanoyl]iso-Glu-KEK-Peg3-
Peg3)]ATILDEQAARDFIAWLIEHKITD;
H[Aib]EGTFTSELATILD[K([19-Carboxy-nonadecanoyl]-isoGlu-KEK-
Peg3)]QAARDFIAWLIEHKITD;
H[Aib]EGSFTSELATILD[K([19-Carboxy-nonadecanoyl]-isoGlu-KEK-
Peg3)]QAARDFIAWLIEHKITD;
H[Aib]EGSFTSELATILD[K([19-Carboxy-nonadecanoyl]-isoGlu-KEK-
Peg3)]QAARDFIAWLIAHKITD;
H[Aib]EGSFTSELATILD[K([19-Carboxy-nonadecanoyl]-isoGlu-KEK-
Peg3)]KAARDFIAWLIEHKITD;
H[Aib]EGSFTSELATILD[K([19-carboxy-nonadecanoyl]iso-Glu-KEK-Peg3-
Peg3)]QAARDFIAWLIEHKITD;
H[Aib]EGSFTSELATILEG[K([19-Carboxy-nonadecanoyl]-isoGlu-KEK-
Peg3)]AARDFIAWLIEHKITD;
H[Aib]EGSFTSELATILDA[K([19-Carboxy-nonadecanoyl]-isoGlu-KEK-
Peg3)]AARDFIAWLIEHKITD;
H[Aib]EGSFTSELATILDA[K([19-carboxy-nonadecanoyl]iso-Glu-KEK-Peg3-
Peg3)]AARDFIAWLIEHKITD;
H[Aib]EGSFTSELATILDEQAA[K([19-Carboxy-nonadecanoyl]-isoGlu-KEK-
Peg3)]DFIAWLIEHKITD;
H[Aib]EGTFTSELATILDEQAA[K([19-Carboxy-nonadecanoyl]-isoGlu-KEK-
Peg3)]DFIAWLIEHKITD;
H[Aib]EGSFTSELATILDEQAA[K([19-carboxy-nonadecanoyl]iso-Glu-KEK-Peg3-
Peg3)]DFIAWLIEHKITD;
H[Aib]EGTFTSELATILDEQAA[K([19-carboxy-nonadecanoyl]iso-Glu-KEK-Peg3-
Peg3)]DFIAWLIEHKITD;
or
H[Aib]EGSFTSELATILDAKAA[K([19-Carboxy-nonadecanoyl]-isoGlu-KEK-
Peg3)]DFIAWLIEHKITD.

The dual agonist may be:

Hy-H[Aib]EGTFSSELATILD[K([17-carboxy-heptadecanoyl]-
isoGlu)]EAARDFIAWLIEHKITD-OH (Compound 1);
Hy-H[Aib]EGSFTSELATILD[K([17-carboxy-heptadecanoyl]-
isoGlu)]EAARDFIAWLIEHKITD-OH (Compound 2);
Hy-H[Aib]EGTFTSELATILD[K([17-carboxy-heptadecanoyl]-
isoGlu)]EAARDFIAWLIEHKITD-OH (Compound 3);
Hy-H[Aib]EGTFSSELATILD[K([17-carboxy-heptadecanoyl]-
isoGlu)]KAARDFIAWLIEHKITD-OH (Compound 4);
Hy-H[Aib]EGSFTSELATILD[K([17-carboxy-heptadecanoyl]-
isoGlu)]KAARDFIAWLIEHKITD-OH (Compound 5);
Hy-H[Aib]EGTFTSELATILD[K([17-carboxy-heptadecanoyl]-
isoGlu)]KAARDFIAWLIEHKITD-OH (Compound 6);
Hy-H[Aib]EGTFSSELATILDG[K([17-carboxy-heptadecanoyl]-
isoGlu)]AARDFIAWLIEHKITD-OH (Compound 7);
Hy-H[Aib]EGSFTSELATILDG[K([17-carboxy-heptadecanoyl]-
isoGlu)]AARDFIAWLIEHKITD-OH (Compound 8);
Hy-H[Aib]EGTFTSELATILDG[K([17-carboxy-heptadecanoyl]-
isoGlu)]AARDFIAWLIEHKITD-OH (Compound 9);
Hy-H[Aib]EGTFSSELATILD[K([17-carboxy-heptadecanoyl]-
isoGlu)]LAARDFIAWLIEHKITD-OH (Compound 10);
Hy-H[Aib]EGSFTSELATILD[K([17-carboxy-heptadecanoyl]-
isoGlu)]LAARDFIAWLIEHKITD-OH (Compound 11);
Hy-H[Aib]EGTFTSELATILD[K([17-carboxy-heptadecanoyl]-
isoGlu)]LAARDFIAWLIEHKITD-OH (Compound 12);
Hy-H[Aib]EGTFSSELATILD[K([17-carboxy-heptadecanoyl]-
isoGlu)]LAARDFIAWLIAHKITD-OH (Compound 13);
Hy-H[Aib]EGSFTSELATILD[K([17-carboxy-heptadecanoyl]-
isoGlu)]LAARDFIAWLIAHKITD-OH (Compound 14);
Hy-H[Aib]EGTFTSELATILD[K([17-carboxy-heptadecanoyl]-
isoGlu)]LAARDFIAWLIAHKITD-OH (Compound 15);
Hy-H[Aib]EGTFTSELATILD[K([17-carboxy-heptadecanoyl]-
isoGlu)]EAARLFIAWLIEHKITD-OH (Compound 16);
Hy-H[Aib]EGTFSSELATILD[K([17-carboxy-heptadecanoyl]-
isoGlu)]QAARDFIAWLIQHKITD-OH (Compound 17);
Hy-H[Aib]EGSFTSELATILD[K([17-carboxy-heptadecanoyl]-
isoGlu)]QAARDFIAWLIQHKITD-OH (Compound 18);
Hy-H[Aib]EGTFTSELATILD[K([17-carboxy-heptadecanoyl]-
isoGlu)]QAARDFIAWLIQHKITD-OH (Compound 19);
Hy-H[Aib]EGTFSSELATILD[K([17-carboxy-heptadecanoyl]-
isoGlu)]QAARDFIAWLIEHKITD-OH (Compound 20);
Hy-H[Aib]EGTFSSELATILD[K([17-carboxy-heptadecanoyl]-
isoGlu)]QAARDFIAWLIAHKITD-OH (Compound 21);
Hy-H[Aib]EGSFTSELATILD[K([17-carboxy-heptadecanoyl]-
isoGlu)]QAARDFIAWLIAHKITD-OH (Compound 22);
Hy-H[Aib]EGTFTSELATILD[K([17-carboxy-heptadecanoyl]-
isoGlu)]QAARDFIAWLIAHKITD-OH (Compound 23);
Hy-H[Aib]EGSFTSELATILD[K([17-carboxy-heptadecanoyl]-
isoGlu)]QAARDFIAWLIEHKITD-OH (Compound 24);
Hy-H[Aib]EGTFTSELATILD[K([17-carboxy-heptadecanoyl]-
isoGlu)]QAARDFIAWLIEHKITD-OH (Compound 25);
Hy-H[Aib]EGSFTSELATILD[K([17-carboxy-heptadecanoyl]-
isoGlu)]QAARDFIAWLIHHKITD-OH (Compound 26);
Hy-H[Aib]EGSFTSELATILD[K([17-carboxy-heptadecanoyl]-
isoGlu)]QAARDFIAWLIYHKITD-OH (Compound 27);
Hy-H[Aib]EGSFTSELATILD[K([17-carboxy-heptadecanoyl]-
isoGlu)]QAARDFIAWLILHKITD-OH (Compound 28);
Hy-H[Aib]EGSFTSELATILD[K([17-carboxy-heptadecanoyl]-
isoGlu)]QAARDFIAWLIKHKITD-OH (Compound 29);
Hy-H[Aib]EGSFTSELATILD[K([17-carboxy-heptadecanoyl]-
isoGlu)]QAARDFIAWLIRHKITD-OH (Compound 30);
Hy-H[Aib]EGSFTSELATILD[K([17-carboxy-heptadecanoyl]-
isoGlu)]QAARDFIAWLISHKITD-OH (Compound 31).
Hy-H[Aib]EGSFTSELATILD[K([Hexadecanoyl]-Ala)]QAARDFIAWLQQHKITD-OH
(Compound 32);
Hy-H[Aib]EGSFTSELATILD[K([17-carboxy-heptadecanoyl]iso-Glu-
Peg3)]QAARDFIAWLYQHKITD-OH (Compound 33);
Hy-H[Aib]EGSFTSELATILD[K([19-carboxy-nonadecanoyl]iso-Glu-Peg3-
Peg3)]QAARDFIAWLKQHKITD-OH (Compound 34);
Hy-H[Aib]EGSFTSELATILD[K([19-carboxy-nonadecanoyl]iso-Lys-Peg3-Peg3-
Peg3)]QAARDFIAWLIQQKITD-OH (Compound 35);
Hy-H[Aib]EGSFTSELATILD[K(Octadecanoyl)]QAARDFIAWLIQYKITD-OH
(Compound 36);
Hy-H[Aib]EGTFSSELSTILE[K(Hexadecanoyl-isoGlu)]QASREFIAWLIAYKITE-OH
(Compound 37);
Hy-H[Aib]EGTFSSELATILDEQAARDFIAWLIAHKITDkkkkkk([17-carboxy-
Heptadecanoyl]-isoGlu)]-[NH2] (Compound 38);
Hy-H[Aib]EGTFTSELATILDEQAARDFIAWLIAHKITDkkkkkk([17-carboxy-
Heptadecanoyl]-isoGlu)]-[NH2] (Compound 39);
Hy-H[Aib]EGSFTSELATILDEQAARDFIAWLIEHKITDkkkkkk([17-carboxy-
Heptadecanoyl]-isoGlu)]-[NH2] (Compound 40);
Hy-H[Aib]EGTFTSELATILD[K([19-Carboxy-nonadecanoyl]-
isoGlu)]QAARDFIAWLIQHKITD-OH (Compound 41);
Hy-H[Aib]EGSFTSE[K([19-carboxy-nonadecanoyl]iso-Glu-Peg3-
Peg3)]ATILDEQAARDFIAWLIEHKITD-OH (Compound 42);
Hy-H[Aib]EGSFTSELATILD[K([19-carboxy-nonadecanoyl]iso-Glu-Peg3-
Peg3)]KAARDFIAWLIEHKITD-OH (Compound 43);
Hy-H[Aib]EGSFTSELATILEG[K([19-carboxy-nonadecanoyl]iso-Glu-Peg3-
Peg3)]AARDFIAWLIEHKITD-OH (Compound 44);
Hy-H[Aib]EGSFTSELATILDEQAA[K([19-carboxy-nonadecanoyl]iso-Glu-Peg3-
Peg3)]DFIAWLIEHKITD-OH (Compound 45);
Hy-H[Aib]EGTFTSELATILDEQAA[K([19-carboxy-nonadecanoyl]iso-Glu-Peg3-
Peg3)]DFIAWLIEHKITD-OH (Compound 46).
Hy-H[Aib]EGTFSSELATILD[K([17-Carboxy-heptadecanoyl]-isoGlu-KEK-
Peg3)]QAARDFIAWLIQHKITD-OH (Compound 47);
Hy-H[Aib]EGTFSSELATILD[K([19-Carboxy-nonadecanoyl]-isoGlu-KEK-
Peg3)]QAARDFIAWLIQHKITD-OH (Compound 48);
Hy-H[Aib]EGTFSSELATILD[K([17-Carboxy-heptadecanoyl]-isoGlu-KEK-
Peg3)]QAARDFIAWLIEHKITD-OH (Compound 49);
Hy-H[Aib]EGTFSSELATILD[K([19-Carboxy-nonadecanoyl]-isoGlu-KEK-
Peg3)]QAARDFIAWLIEHKITD-OH (Compound 50);
Hy-H[Aib]EGTFTSELATILD[K([19-Carboxy-nonadecanoyl]-isoGlu-
KEK)]QAARDFIAWLIQHKITD-OH (Compound 51);
Hy-H[Aib]EGTFTSELATILD[K([19-Carboxy-nonadecanoyl]-isoGlu-KEK-
Peg3)]QAARDFIAWLIQHKITD-OH (Compound 52);
Hy-H[Aib]EGSFTSE[K([19-Carboxy-nonadecanoyl]-isoGlu-KEK-
Peg3)]ATILDEQAARDFIAWLIEHKITD-OH (Compound 53);
Hy-H[Aib]EGTFTSE[K([19-Carboxy-nonadecanoyl]-isoGlu-KEK-
Peg3)]ATILDEQAARDFIAWLIEHKITD-OH (Compound 54);
Hy-H[Aib]EGSFTSE[K([19-carboxy-nonadecanoyl]iso-Glu-KEK-Peg3-
Peg3)]ATILDEQAARDFIAWLIEHKITD-OH (Compound 55);
Hy-H[Aib]EGTFTSELATILD[K([19-Carboxy-nonadecanoyl]-isoGlu-KEK-
Peg3)]QAARDFIAWLIEHKITD-OH (Compound 56);
Hy-H[Aib]EGSFTSELATILD[K([19-Carboxy-nonadecanoyl]-isoGlu-KEK-
Peg3)]QAARDFIAWLIEHKITD-OH (Compound 57);
Hy-H[Aib]EGSFTSELATILD[K([19-Carboxy-nonadecanoyl]-isoGlu-KEK-
Peg3)]QAARDFIAWLIAHKITD-OH (Compound 58);
Hy-H[Aib]EGSFTSELATILD[K([19-Carboxy-nonadecanoyl]-isoGlu-KEK-
Peg3)]KAARDFIAWLIEHKITD-OH (Compound 59);
Hy-H[Aib]EGSFTSELATILD[K([19-carboxy-nonadecanoyl]iso-Glu-KEK-Peg3-
Peg3)]QAARDFIAWLIEHKITD-OH (Compound 60);
Hy-H[Aib]EGSFTSELATILEG[K([19-Carboxy-nonadecanoyl]-isoGlu-KEK-
Peg3)]AARDFIAWLIEHKITD-OH (Compound 61);
Hy-H[Aib]EGSFTSELATILDA[K([19-Carboxy-nonadecanoyl]-isoGlu-KEK-
Peg3)]AARDFIAWLIEHKITD-OH (Compound 62);
Hy-H[Aib]EGSFTSELATILDA[K([19-carboxy-nonadecanoyl]iso-Glu-KEK-Peg3-
Peg3)]AARDFIAWLIEHKITD-OH (Compound 63);
Hy-H[Aib]EGSFTSELATILDEQAA[K([19-Carboxy-nonadecanoyl]-isoGlu-KEK-
Peg3)]DFIAWLIEHKITD-OH (Compound 64);
Hy-H[Aib]EGTFTSELATILDEQAA[K([19-Carboxy-nonadecanoyl]-isoGlu-KEK-
Peg3)]DFIAWLIEHKITD-OH (Compound 65);
Hy-H[Aib]EGSFTSELATILDEQAA[K([19-carboxy-nonadecanoyl]iso-Glu-KEK-Peg3-
Peg3)]DFIAWLIEHKITD-OH (Compound 66);
Hy-H[Aib]EGTFTSELATILDEQAA[K([19-carboxy-nonadecanoyl]iso-Glu-KEK-Peg3-
Peg3)]DFIAWLIEHKITD-OH (Compound 67); or
Hy-H[Aib]EGSFTSELATILDAKAA[K([19-Carboxy-nonadecanoyl]-isoGlu-KEK-
Peg3)]DFIAWLIEHKITD-oH (Compound 68).

In a preferred embodiment of the invention as described herein the dual agonist is Hy-H[Aib]EGSFTSELATILD[K([17-carboxy-heptadecanoyl]-isoGlu)]QAARDFIAWLIQHKITD-OH (Compound 18).

In an alternative aspect the GLP-1/GLP-2 dual agonist is a compound represented by the formula:

R1—X*-U-R2

wherein:

R1 is hydrogen (Hy), C1-4 alkyl (e.g. methyl), acetyl, formyl, benzoyl or trifluoroacetyl;

R2 is NH2 or OH;

X* is a peptide of formula I:

(I)
H-X2-EG-X5-F-X7-X8-E-X10-X11-TIL-X15-X16-X17-A-
X19-X20-X21-FI-X24-WL-X27-X28-X29-KIT-X33

wherein

X2 is Aib or G;

X5 is S or T;

X7 is S or T;

X8 is S, E or D;

X10 is L, M or V;

X11 is A, N or S;

X15 is D or E

X16 is E, A or G;

X17 is Q, E, L or K;

X19 is A, V or S;

X20 is R or K;

X21 is D, L or E;

X24 is A, N or S;

X27 is I, Y, Q, H or K;

X28 is A, E, H, Y, L, K, Q, R or S;

X29 is H, Y, K or Q;

X33 is D or E;

U is absent or a sequence of 1-15 residues, each independently selected from K and k;

and wherein at least one of X5 and X7 is T;

or a pharmaceutically acceptable salt or solvate thereof.

The agonist combination according to the invention may be in the form of a pharmaceutically acceptable salt or solvate, such as a pharmaceutically acceptable acid addition salt.

The agonist combination may be in the form of a composition, which, for example, may comprise the agonist combination, or a pharmaceutically acceptable salt or solvate thereof, together with a carrier, excipient or vehicle. The carrier may be a pharmaceutically acceptable carrier.

The composition may be a pharmaceutical composition. The pharmaceutical composition may be formulated as a liquid suitable for administration by injection or infusion. It may be formulated to achieve slow release of the agonist combination.

The agonist combination may be administered at a dose of about 0.1 pmol/kg to 500 μmol/kg body weight.

The agonist combination may be administered for 1, 2, 3, 4, 5, 6, or 7 or more days after surgery.

The agonist combination may also be administered to said patient prior to said surgical resection.

In one aspect of the invention, about 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, or 20.0% or more, such as about 30, 40, 50, 60, 70, 80 or 90% of the bowel of said patient is removed.

In a further aspect the patient may receive enteral nutrition after said surgical resection, for example within about 48 hours of said surgical resection.

In one aspect said patient has short bowel syndrome secondary to one or more of digestion disorders, malabsorption syndromes, short-gut syndrome, cul-de-sac syndrome, inflammatory bowel disease, celiac sprue (for example arising from gluten induced enteropathy or celiac disease), tropical sprue, hypogammaglobulinemic sprue, enteritis, ulcerative colitis, small intestine damage Crohn's disease, mesenteric infarction, volvulus, multiple strictures due to adhesions or radiation, vascular ischemia, necrotising enteral colitis (NEC), intestinal malformations, intestinal atresia, bowel cancer, surgical complications, acute injury, for example a stab injury.

In one aspect the composition may be effective to increase villus growth, increase intestinal length, increase cell growth (and/or decrease cell death) and/or increase intestinal weight.

A further aspect provides a therapeutic kit comprising an agonist combination according to the invention for use in the treatment of a patient who has undergone surgical resection of the bowel, wherein said agonist combination is administered to said patient within 7 days of said surgical resection.

### FIGURE LEGENDS

FIG. 1: Anatomy of bowel and illustration of resection. In wild-type mice either a 40% ileum plus cecum resection (ICR) for animals then allocated to the vehicle or compound 18 (Cpd 18) treated groups was carried out, or for the sham group an anastomosis was performed.

FIG. 2: The initial weight of the animals for sham (n=9), Vehicle (n=12) and Cpd 18 (n=15).

FIG. 3: The initial age of the animals sham (n=9), Vehicle (n=12) and Cpd 18 (n=16).

FIG. 4: The resection length in cm for sham (n=9), Vehicle (n=12) and Cpd 18 (n=16).

FIG. 5: Post operative survival of animals for sham (n=11), Vehicle (n=24) and Cpd 18 (n=26).

FIG. 6: Post operative body weight of animals (relative to initial weight) for sham (n=9), Vehicle (n=12) and Cpd 18 (n=14).

FIG. 7: Post operative dietary intake for sham (n=9), Vehicle (n=12) and Cpd 18 (n=14).

FIG. 8: Mucosal height for sham (n=4), Vehicle (n=4) and Cpd 18 (n=6).

FIG. 9: Bowel diameter for sham (n=4), Vehicle (n=4) and Cpd 18 (n=6).

FIG. 10: Wet weight per 2 cm of intestine for sham (n=6), Vehicle (n=8) and Cpd 18 (n=10).

FIG. 11: Dry weight per 2 cm of intestine for sham (n=6), Vehicle (n=8) and Cpd 18 (n=10).

FIG. 12: Water content in % per 2 cm of intestine for sham (n=6), Vehicle (n=8) and Cpd 18 (n=10).

FIG. 13: Histology of intestine for sham animals.

FIG. 14: Histology of intestine for animals treated with Vehicle.

FIG. 15: Histology of intestine for animals treated with Cpd 18.

FIG. 16: Gross morphology of the jejenum (horizontal) and colon (vertical) from sham animal (upper), animal treated with Cpd 18 (middle) and Vehicle treated animal (lower).

### DETAILED DESCRIPTION OF THE INVENTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well known and commonly used in the art.

All patents, published patent applications and non-patent publications referred to in this application are specifically incorporated by reference herein. In case of conflict, the present specification, including its specific definitions, will control.

Each embodiment of the invention described herein may be taken alone or in combination with one or more other embodiments of the invention.

### Definitions

Unless specified otherwise, the following definitions are provided for specific terms which are used in the present written description.

Throughout this specification, the word “comprise”, and grammatical variants thereof, such as “comprises” or “comprising”, will be understood to imply the inclusion of a stated integer or component, or group of integers or components, but not the exclusion of any other integer or component, or group of integers or components.

The singular forms “a,” “an,” and “the” include the plurals unless the context clearly dictates otherwise.

The term “including” is used to mean “including but not limited to”. “Including” and “including but not limited to” may be used interchangeably.

The terms “patient”, “subject” and “individual” may be used interchangeably and refer to either a human or a non-human animal. These terms include mammals such as humans, primates, livestock animals (e.g., bovines and porcines), companion animals (e.g., canines and felines) and rodents (e.g., mice and rats). In one aspect the patient is a human.

The term “solvate” in the context of the present invention refers to a complex of defined stoichiometry formed between a solute (in casu, a peptide or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

The term “agonist” as employed in the context of the invention refers to a substance (ligand) that activates the receptor type in question.

Throughout the present description and claims the conventional three-letter and one-letter codes for naturally occurring amino acids are used, i.e.

A (Ala), G (Gly), L (Leu), I (Ile), V (Val), F (Phe), W (Trp), S (Ser), T (Thr), Y (Tyr), N (Asn), Q (Gin), D (Asp), E (Glu), K (Lys), R (Arg), H (His), M (Met), C (Cys) and P (Pro);

as well as generally accepted three-letter codes for other α-amino acids, such as sarcosine (Sar), norleucine (Nle), α-aminoisobutyric acid (Aib), 2,3-diaminopropanoic acid (Dap), 2,4-diaminobutanoic acid (Dab) and 2,5-diaminopentanoic acid (ornithine; Orn). Such other α-amino acids may be shown in square brackets “[ ]” (e.g. “[Aib]”) when used in a general formula or sequence in the present specification, especially when the rest of the formula or sequence is shown using the single letter code. Unless otherwise specified, amino acid residues in peptides of the invention are of the L-configuration. However, D-configuration amino acids may be incorporated. In the present context, an amino acid code written with a small letter represents the D-configuration of said amino acid, e.g. “k” represents the D-configuration of lysine (K).

Among sequences disclosed herein are sequences incorporating a “Hy-”moiety at the amino terminus (N-terminus) of the sequence, and either an “—OH” moiety or an “—NH₂” moiety at the carboxy terminus (C-terminus) of the sequence. In such cases, and unless otherwise indicated, a “Hy-” moiety at the N-terminus of the sequence in question indicates a hydrogen atom [i.e. R¹=hydrogen=Hy in the general formulas; corresponding to the presence of a free primary or secondary amino group at the N-terminus], while an “—OH” or an “—NH₂” moiety at the C-terminus of the sequence indicates a hydroxy group [e.g. R²=OH in general formulas; corresponding to the presence of a carboxy (COOH) group at the C-terminus] or an amino group [e.g. R²=[NH₂] in the general formulas; corresponding to the presence of an amido (CONH₂) group at the C-terminus], respectively. In each sequence of the invention, a C-terminal “—OH” moiety may be substituted for a C-terminal “—NH₂” moiety, and vice-versa.

“Percent (%) amino acid sequence identity” with respect to the GLP-2 polypeptide sequences is defined as the percentage of amino acid residues in a candidate sequence that are identical to the amino acid residues in the wild-type (human) GLP-2 sequence, after aligning the sequences and introducing gaps, if necessary, to achieve the maximum percent sequence identity, and not considering any conservative substitutions as part of the sequence identity. Sequence alignment can be carried out by the skilled person using techniques well known in the art, for example using publicly available software such as BLAST, BLAST2 or Align software. For examples, see Altschul et al., Methods in Enzymology 266: 460-480 (1996) or Pearson et al., Genomics 46: 24-36, 1997.

The percentage sequence identities used herein in the context of the present invention may be determined using these programs with their default settings. More generally, the skilled worker can readily determine appropriate parameters for determining alignment, including any algorithms needed to achieve maximal alignment over the full length of the sequences being compared.

### GLP-1 and GLP-2 Agonists

The terms “GLP-1 agonist” and “GLP-1 receptor agonist” are used interchangeably herein and have the same meaning. A GLP-1 agonist/GLP-1 receptor agonist has agonist activity at the GLP-1 receptor, e.g. the human GLP-1 receptor.

The terms “GLP-2 agonist” and “GLP-2 receptor agonist” are used interchangeably herein and have the same meaning. A GLP-2 agonist/GLP-2 receptor agonist has agonist activity at the GLP-2 receptor, e.g. the human GLP-2 receptor.

Suitable GLP-1 agonists will be known to one skilled in the art in this field. For example, the GLP-1 agonist may be selected from native GLP-1, a GLP-1 analog, such as Liraglutide (EP1687019, Novo Nordisk A/S), Lixisenatide, Dulaglutide, Semaglutide or Exenatide (Byetta/Bydureon, AstraZeneca/Amylin Pharmaceuticals Inc), and a synthetic GLP-1. In one aspect the GLP-1 agonist may be selected from Bydureon (Exenatide) AstraZeneca—taken once weekly, Byetta (Exenatide) AstraZeneca—taken twice daily, Lyxumia (Lixisenatide) Sanofi—taken once daily, Trulicity (Dulaglutide) Eli Lilly—taken once weekly, Victoza (Liraglutide) Novo Nordisk—taken once daily, and Rybelsus (Semaglutide) Novo Nordisk—taken once weekly.

Suitable GLP-2 agonists will be known to one skilled in the art in this field. For example, the GLP-2 agonist may be selected from native GLP-2, a GLP-2 analog, such as Teduglutide (WO1997039031, Shire NPS Pharmaceuticals, Takeda), Glepaglutide (WO2006117565, Zealand Pharma A/S), BC-GLP-2 (WO2019086559, Adocia), HM15912 (WO2019066586, Hanmi Pharmaceuticals), NB1002 (Naia Pharmaceuticals) or Apraglutide (WO2011050174, Vectivbio), a synthetic GLP-2 and a GLP-2 peptibody such as e.g. SHP681 (WO2019040399, Shire NPS Pharmaceuticals, Takeda).

In one aspect of the invention, the combination of a GLP-1 agonist and a GLP-2 agonist comprises a combination of Teduglutide and Exenatide Teduglutide and Liraglutide, Glepaglutide and Semaglutide, or a combination of Glepaglutide and Exenatide.

### Dual Agonist Compounds

The terms “GLP-1/GLP-2 dual agonist” and “GLP-1/GLP-2 dual receptor agonist” are used interchangeably herein and have the same meaning. The dual agonists/dual receptor agonists have agonist activity at both of the GLP-1 and GLP-2 receptors, e.g. the human GLP-1 and GLP-2 receptors.

The dual agonist has at least one GLP-1 and at least one GLP-2 biological activity.

Exemplary GLP-1 physiological activities include reducing rate of intestinal transit, reducing rate of gastric emptying, reducing appetite, food intake or body weight, and improving glucose control and glucose tolerance. Exemplary GLP-2 physiological activities include causing an increase in intestinal mass (e.g. of small intestine or colon), intestinal repair, and improving intestinal barrier function (i.e. reducing permeability of the intestine). These parameters can be assessed in in vivo assays in which the mass and the permeability of the intestine, or a portion thereof, is determined after a test animal has been treated with a dual agonist.

The dual agonists have agonist activity at the GLP-1 and GLP-2 receptors, e.g. the human GLP-1 and GLP-2 receptors. EC₅₀ values for in vitro receptor agonist activity may be used as a numerical measure of agonist potency at a given receptor. An EC50 value is a measure of the concentration (e.g. mol/L) of a compound required to achieve half of that compound's maximal activity in a particular assay. A compound having a numerical EC₅₀ at a particular receptor which is lower than the EC₅₀ of a reference compound in the same assay may be considered to have higher potency at that receptor than the reference compound.

### GLP-1 Activity

In some embodiments, the dual agonist has an EC₅₀ at the GLP-1 receptor (e.g. the human GLP-1 receptor) which is below 2.0 nM, below 1.5 nM, below 1.0 nM, below 0.9 nM, below 0.8 nM, below 0.7 nM, below 0.6 nM, below 0.5 nM, below 0.4 nM, below 0.3 nM, below 0.2 nM, below 0.1 nM, below 0.09 nM, below 0.08 nM, below 0.07 nM, below 0.06 nM, below 0.05 nM, below 0.04 nM, e.g. when assessed using the GLP-1 receptor potency assay described in Example 2 of WO2018/104561.

In some embodiments, the dual agonist has an EC₅₀ at the GLP-1 receptor which is between 0.005 and 2.5 nM, between 0.01 nM and 2.5 nM, between 0.025 and 2.5 nM, between 0.005 and 2.0 nM, between 0.01 nM and 2.0 nM, between 0.025 and 2.0 nM, between 0.005 and 1.5 nM, between 0.01 nM and 1.5 nM, between 0.025 and 1.5 nM, between 0.005 and 1.0 nM, between 0.01 nM and 1.0 nM, between 0.025 and 1.0 nM, between 0.005 and 0.5 nM, between 0.01 nM and 0.5 nM, between 0.025 and 0.5 nM, between 0.005 and 0.25 nM, between 0.01 nM and 0.25 nM, between 0.025 and 0.25 nM, e.g. when assessed using the GLP-1 receptor potency assay described in Example 2 of WO2018/104561.

An alternative measure of GLP-1 agonist activity may be derived by comparing the potency of a dual agonist with the potency of a known (or reference) GLP-1 agonist when both are measured in the same assay. Thus the relative potency at the GLP-1 receptor may be defined as:

[EC₅₀(reference agonist)]/[EC₅₀(dual agonist)].

Thus a value of 1 indicates that the dual agonist and reference agonist have equal potency, a value of >1 indicates that the dual agonist has higher potency (i.e. lower EC₅₀) than the reference agonist, and a value of <1 indicates that the dual agonist has lower potency (i.e. higher EC₅₀) than the reference agonist.

The reference GLP-1 agonist may, for example, be human GLP-1(7-37), liraglutide (NN2211; Victoza), or Exendin-4, but is preferably liraglutide.

Typically the relative potency will be between 0.001 and 100, e.g.

between 0.001 and 10, between 0.001 and 5, between 0.001 and 1, between 0.001 and 0.5, between 0.001 and 0.1, between 0.001 and 0.05, or between 0.001 and 0.01;

between 0.01 and 10, between 0.01 and 5, between 0.01 and 1, between 0.01 and 0.5, between 0.01 and 0.1, or between 0.01 and 0.05;

between 0.05 and 10, between 0.05 and 5, between 0.05 and 1, between 0.05 and 0.5, or between 0.05 and 0.1;

between 0.1 and 10, between 0.1 and 5, between 0.1 and 1, or between 0.1 and 0.5;

between 0.5 and 10, between 0.5 and 5, or between 0.5 and 1;

between 1 and 10, or between 1 and 5;

or between 5 and 10.

The dual agonist according to the invention may have higher potency at the GLP-1 receptor (e.g. the human GLP-1 receptor) than wild type human GLP-2 (hGLP-2 (1-33)) or [Gly2]-hGLP-2 (1-33) (i.e. human GLP-2 having glycine at position 2, also known as teduglutide). Thus, the relative potency of the dual agonists at the GLP-1 receptor compared to hGLP-2 (1-33) or teduglutide is greater than 1, typically greater than 5 or greater than 10, and may be up to 100, up to 500, or even higher.

### GLP-2 Activity

In some embodiments, the dual agonist has an EC₅₀ at the GLP-2 receptor (e.g. the human GLP-2 receptor) which is below 2.0 nM, below 1.5 nM, below 1.0 nM, below 0.9 nM, below 0.8 nM, below 0.7 nM, below 0.6 nM, below 0.5 nM, below 0.4 nM, below 0.3 nM, below 0.2 nM, below 0.1 nM, below 0.09 nM, below 0.08 nM, below 0.07 nM, below 0.06 nM, below 0.05 nM, below 0.04 nM, below 0.03 nM, below 0.02 nM, or below 0.01 nM, e.g. when assessed using the GLP-1 receptor potency assay described in Example 2 of WO2018/104561.

In some embodiments, the dual agonist has an EC₅₀ at the GLP-2 receptor which is between 0.005 and 2.0 nM, between 0.01 nM and 2.0 nM, between 0.025 and 2.0 nM, between 0.005 and 1.5 nM, between 0.01 nM and 1.5 nM, between 0.025 and 1.5 nM, between 0.005 and 1.0 nM, between 0.01 nM and 1.0 nM, between 0.025 and 1.0 nM, between 0.005 and 0.5 nM, between 0.01 nM and 0.5 nM, between 0.025 and 0.5 nM, between 0.005 and 0.25 nM, between 0.01 nM and 0.25 nM, between 0.025 and 0.25 nM, e.g. when assessed using the GLP-2 receptor potency assay described in the Example 2 of WO2018/104561.

An alternative measure of GLP-2 agonist activity may be derived by comparing the potency of a dual agonist with the potency of a known (or reference) GLP-2 agonist when both are measured in the same assay. Thus the relative potency at the GLP-2 receptor may be defined as:

[EC₅₀(reference agonist)]/[EC₅₀(dual agonist)].

Thus a value of 1 indicates that the dual agonist and reference agonist have equal potency, a value of >1 indicates that the dual agonist has higher potency (i.e. lower EC₅₀) than the reference agonist, and a value of <1 indicates that the dual agonist has lower potency (i.e. higher EC₅₀) than the reference agonist.

The reference GLP-2 agonist may, for example, be human GLP-2(1-33) or teduglutide ([Gly2]-hGLP-2 (1-33)), but is preferably teduglutide. Typically the relative potency will be between 0.001 and 100, e.g.

between 0.001 and 10, between 0.001 and 5, between 0.001 and 1, between 0.001 and 0.5, between 0.001 and 0.1, between 0.001 and 0.05, or between 0.001 and 0.01;

between 0.01 and 10, between 0.01 and 5, between 0.01 and 1, between 0.01 and 0.5, between 0.01 and 0.1, or between 0.01 and 0.05;

between 0.05 and 10, between 0.05 and 5, between 0.05 and 1, between 0.05 and 0.5, or between 0.05 and 0.1;

between 0.1 and 10, between 0.1 and 5, between 0.1 and 1, or between 0.1 and 0.5;

between 0.5 and 10, between 0.5 and 5, or between 0.5 and 1;

between 1 and 10, or between 1 and 5;

or between 5 and 10.

The dual agonists according to the invention may have higher potency at the GLP-2 receptor (e.g. the human GLP-2 receptor) than human GLP-1(7-37), liraglutide (NN2211; Victoza), or Exendin-4. Thus, the relative potency of the dual agonists at the GLP-2 receptor compared to human GLP-1(7-37), liraglutide (NN2211; Victoza), or Exendin-4 is greater than 1, typically greater than 5 or greater than 10, and may be up to 100, up to 500, or even higher (if the reference GLP-1 agonist even exerts detectable activity at the GLP-2 receptor).

It will be understood that the absolute potencies of the dual agonists at each receptor are much less important than the balance between the GLP-1 and GLP-2 agonist activities. Thus it is perfectly acceptable for the absolute GLP-1 or GLP-2 potency to be lower than that of known agonists at those receptors, as long as the dual agonist compound exerts acceptable relative levels of potency at both receptors. Any apparent deficiency in absolute potency can be compensated by an increased dose if required.

### Substituents

The dual agonist may contain a residue Ψ which comprises a residue of Lys, Arg, Orn, Dap or Dab in which the side chain is conjugated to a substituent Z¹— or Z¹—Z²— wherein Z¹ represents a moiety CH₃—(CH₂)₁₀₋₂₂—(CO)— or HOOC—(CH₂)₁₀₋₂₂—(CO)— and Z² when present represents a spacer.

The spacer Z² is selected from —Z^{S1}—, —Z^{S1}—Z^{S2}—, —Z^{S2}—Z^{S1}, —Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, —Z^{S2}Z^{S3}Z^{S2}— wherein

Z^{S1} is isoGlu, β-Ala, isoLys, or 4-aminobutanoyl;

Z^{S2} is -(Peg3)ₘ- where m is 1, 2, or 3; and

Z^{S3}— is a peptide sequence of 1-6 amino acid units selected from the group consisting of A, L, S, T, Y, Q, D, E, K, k, R, H, F and G.

In some embodiments, Z² is a spacer of the formula —Z^{S1}—, —Z^{S1}—Z^{S2}—, —Z^{S2}—Z^{S1}, or Z^{S2}, where —Z^{S1}— is isoGlu, β-Ala, isoLys, or 4-aminobutanoyl; and —Z^{S2}— is -(Peg3)ₘ- where m is 1, 2, or 3.

Without wishing to be bound by theory, it is believed that the hydrocarbon chain of Z¹ binds albumin in the blood stream, thus shielding the dual agonists of the present invention from enzymatic degradation, which can enhance the half-life of the dual agonists.

The substituent may also modulate the potency of the dual agonists, with respect to the GLP-2 receptor and/or the GLP-1 receptor.

The substituent Z¹— or Z¹—Z²— is conjugated to the functional group at the distal end of the side-chain from the alpha-carbon of the relevant amino acid residue. The normal ability of the amino acid (Lys, Arg, Orn, Dab, Dap) side-chain in question to participate in interactions mediated by that functional group (e.g. intra- and inter-molecular interactions) may therefore be reduced or completely eliminated by the presence of the substituent. Thus, the overall properties of the dual agonist may be relatively insensitive to changes in the actual amino acid conjugated to the substituent. Consequently, it is believed that any of the residues Lys, Arg, Orn, Dab, or Dap may be present at any position where Ψ is permitted. However, in certain embodiments, it may be advantageous that the amino acid to which the substituent is conjugated is Lys or Orn.

The moiety Z¹ may be covalently bonded to the functional group in the amino acid side-chain, or alternatively may be conjugated to the amino acid side-chain functional group via a spacer Z².

The term “conjugated” is used here to describe the covalent attachment of one identifiable chemical moiety to another, and the structural relationship between such moieties. It should not be taken to imply any particular method of synthesis.

The bonds between Z^{S1}, Z^{S2}, Z^{S3}, and the amino acid side chain to which the substituent is bound (collectively referred to herein as Ψ) are peptidic. In other words, the units may be joined by amide condensation reactions.

Z¹ comprises a hydrocarbon chain having from 10 to 24 carbon (C) atoms, such as from 10 to 22 C atoms, e.g. from 10 to 20 C atoms. Preferably, it has at least 10 or at least 11 C atoms, and preferably it has 20 C atoms or fewer, e.g. 18 C atoms or fewer. For example, the hydrocarbon chain may contain 12, 13, 14, 15, 16, 17, 18, 19 or 20 carbon atoms. For example, it may contain 18 or 20 carbon atoms.

In some embodiments, Z¹ is a group selected from dodecanoyl, tetradecanoyl, hexadecanoyl, octadecanoyl and eicosanoyl, preferably hexadecanoyl, octadecanoyl or eicosanoyl, more preferably octadecanoyl or eicosanoyl.

Alternative Z¹ groups are derived from long-chain saturated α,ω-dicarboxylic acids of formula HOOC—(CH₂)₁₂₋₂₂—COOH, preferably from long-chain saturated α,ω-dicarboxylic acids having an even number of carbon atoms in the aliphatic chain. For example, Z¹ may be:

13-carboxytridecanoyl, i.e. HOOC—(CH₂)₁₂—(CO)—;

15-carboxypentadecanoyl, i.e. HOOC—(CH₂)₁₄—(CO)—;

17-carboxyheptadecanoyl, i.e. HOOC—(CH₂)₁₆—(CO)—;

19-carboxynonadecanoyl, i.e. HOOC—(CH₂)₁₈—(CO)—; or

21-carboxyheneicosanoyl, i.e. HOOC—(CH₂)₂₀—(CO)—.

As mentioned above, Z¹ may be conjugated to the amino acid side-chain by a spacer Z². When present, the spacer is attached to Z¹ and to the amino acid side-chain.

The spacer Z² has the —Z^{S1}—, —Z^{S1}—Z^{S2}—, —Z^{S2}—Z^{S1}, —Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, —Z^{S2}Z^{S3}Z^{S2}— where

—Z^{S1}— is isoGlu, β-Ala, isoLys, or 4-aminobutanoyl;

—Z^{S2}— is -(Peg3)ₘ- where m is 1, 2, or 3; and

—Z^{S3}— is a peptide sequence of 1-6 amino acid units independently selected from the group consisting of A (Ala), L (Leu), S (Ser), T (Thr), Y (Tyr), Q (Gln), D (Asp), E (Glu), K (L-Lys), k (D-Lys), R (Arg), H (His), F (Phe) and G (Gly).

The terms “isoGlu” and “isoLys” indicate residues of amino acids which participate in bonds via their side chain carboxyl or amine functional groups. Thus isoGlu participates in bonds via its alpha amino and side chain carboxyl group, while isoLys participates via its carboxyl and side chain amino groups. In the context of the present specification, the terms “γ-Glu” and “isoGlu” are used interchangeably.

The term Peg3 is used to refer to an 8-amino-3,6-dioxaoctanoyl group.

Z^{S3} may, for example, be 3 to 6 amino acids in length, i.e. 3, 4, 5 or 6 amino acids in length.

In some embodiments, the amino acids of Z^{S3} are independently selected from K, k, E, A, T, I and L, e.g. from K, k, E and A, e.g. from K, k and E.

Typically Z^{S3} includes at least one charged amino acid (K, k, R or E, e.g. K, k or E) and preferably two or more charged amino acids. In some embodiments it includes at least 2 positively charged amino acids (K, k or R, especially K or k), or at least 1 positively charged amino acid (K, k or R, especially K or k) and at least one negatively charged amino acid (E). In some embodiments, all amino acid residues of Z^{S3} are charged. For example, Z^{S3} may be a chain of alternately positively and negatively charged amino acids.

Examples of Z^{S3} moieties include KEK, EKEKEK, kkkkkk, EkEkEk, AKAAEK, AKEKEK and ATILEK.

Without being bound by theory, it is believed that the incorporation of Z^{S3} into the linker between the fatty acid chain and the peptide backbone may increase the half-life of the dual agonist by enhancing its affinity for serum albumin.

In some embodiments, —Z²— is —Z^{S1}— or —Z^{S1}—Z^{S2}—; in other words, —Z²— is selected from:

isoGlu(Peg3)₀₋₃;

β-Ala(Peg3)₀₋₃;

isoLys(Peg3)₀₋₃; and

4-aminobutanoyl(Peg3)₀₋₃.

Thus, certain examples of substituents Z¹— include

[Dodecanoyl], [Tetradecanoyl], [Hexadecanoyl], [Octadecanoyl], [Eicosanoyl], [13-Carboxy-tridecanoyl], [15-Carboxy-pentadecanoyl], [17-Carboxy-heptadecanoyl], [19-Carboxy-nonadecanoyl], [21-carboxy-heneicosanoyl].

More broadly, —Z²— may be —Z^{S1}—, —Z^{S1}—Z^{S2}—, —Z^{S3}—Z^{S1}—, —Z^{S1}—Z^{S3}—, Z^{S1}—Z^{S3}—Z^{S2}—, —Z^{S3}—Z^{S2}—Z^{S1}— or Z^{S3}—. Thus, —Z²— may be selected from the group consisting of:

isoGlu(Peg3)₀₋₃;

β-Ala(Peg3)₀₋₃;

isoLys(Peg3)₀₋₃;

4-aminobutanoyl(Peg3)₀₋₃;

isoGlu(KEK)(Peg3)₀₋₃;

β-Ala(KEK)(Peg3)₀₋₃;

isoLys(KEK)(Peg3)₀₋₃;

4-aminobutanoyl(KEK)(Peg3)₀₋₃;

KEK(isoGlu);

KEK(β-Ala);

KEK(isoLys);

KEK(4-aminobutanoyl);

isoGlu(KEK);

β-Ala(KEK);

isoLys(KEK);

4-aminobutanoyl(KEK);

KEK(isoGlu)(Peg3)₀₋₃;

KEK(β-Ala)(Prg3)₀₋₃;

KEK(isoLys)(Peg3)₀₋₃; and

KEK(4-aminobutanoyl)(Peg3)₀₋₃;

Certain examples of substituents Z¹—Z²— include:

[Dodecanoyl]-isoGlu, [Tetradecanoyl]-isoGlu, [Hexadecanoyl]-isoGlu, [Octadecanoyl]-isoGlu, [Eicosanoyl]-isoGlu,

[Hexadecanoyl]-βAla, [Octadecanoyl]-βAla, [Eicosanoyl]-βAla, [Tetradecanoyl]-βAla, [Dodecanoyl]-βAla,

[Dodecanoyl]-isoGlu-Peg3, [Tetradecanoyl]-isoGlu-Peg3, [Hexadecanoyl]-isoGlu-Peg3, [Octadecanoyl]-isoGlu-Peg3, [Eicosanoyl]-isoGlu-Peg3,

[Dodecanoyl]-βAla-Peg3, [Tetradecanoyl]-βAla-Peg3, [Hexadecanoyl]-βAla-Peg3, [Octadecanoyl]-βAla-Peg3, [Eicosanoyl]-βAla-Peg3,

[Dodecanoyl]-isoGlu-Peg3-Peg3, [Tetradecanoyl]-isoGlu-Peg3-Peg3, [Hexadecanoyl]-isoGlu-Peg3-Peg3, [Octadecanoyl]-isoGlu-Peg3-Peg3, [Eicosanoyl]-isoGlu-Peg3-Peg3,

[Dodecanoyl]-βAla-Peg3-Peg3, [Tetradecanoyl]-βAla-Peg3-Peg3, [Hexadecanoyl]-βAla-Peg3-Peg3, [Octadecanoyl]-βAla-Peg3-Peg3, [Eicosanoyl]-βAla-Peg3-Peg3,

[Dodecanoyl]-isoGlu-Peg3-Peg3-Peg3, [Tetradecanoyl]-isoGlu-Peg3-Peg3-Peg3, [Hexadecanoyl]-isoGlu-Peg3-Peg3-Peg3, [Octadecanoyl]-isoGlu-Peg3-Peg3-Peg3, [Eicosanoyl]-isoGlu-Peg3-Peg3-Peg3,

[Dodecanoyl]-βAla-Peg3-Peg3-Peg3, [Tetradecanoyl]-βAla-Peg3-Peg3-Peg3, [Hexadecanoyl]-βAla-Peg3-Peg3-Peg3, [Octadecanoyl]-βAla-Peg3-Peg3-Peg3, [Eicosanoyl]-βAla-Peg3-Peg3-Peg3,

[Dodecanoyl]-isoLys, [Tetradecanoyl]-isoLys, [Hexadecanoyl]-isoLys, [Octadecanoyl]-isoLys, [Eicosanoyl]-isoLys,

[Hexadecanoyl]-[4-aminobutanoyl], [Octadecanoyl]-[4-aminobutanoyl], [Eicosanoyl]-[4-aminobutanoyl], [Tetradecanoyl]-[4-aminobutanoyl], [Dodecanoyl]-[4-aminobutanoyl],

[Dodecanoyl]-isoLys-Peg3, [Tetradecanoyl]-isoLys-Peg3, [Hexadecanoyl]-isoLys-Peg3, [Octadecanoyl]-isoLys-Peg3, [Eicosanoyl]-isoLys-Peg3,

[Dodecanoyl]-[4-aminobutanoyl]-Peg3, [Tetradecanoyl]- [4-aminobutanoyl]-Peg3, [Hexadecanoyl]-[4-aminobutanoyl]-Peg3,

[Octadecanoyl]-[4-aminobutanoyl]-Peg3, [Eicosanoyl]-[4-aminobutanoyl]-Peg3,

[Dodecanoyl]-isoLys-Peg3-Peg3, [Tetradecanoyl]-isoLys-Peg3-Peg3, [Hexadecanoyl]-isoLys-Peg3-Peg3, [Octadecanoyl]-isoLys-Peg3-Peg3, [Eicosanoyl]-isoLys-Peg3-Peg3,

[Dodecanoyl]-[4-aminobutanoyl]-Peg3-Peg3, [Tetradecanoyl]-[4-aminobutanoyl]-Peg3-Peg3, [Hexadecanoyl]-[4-aminobutanoyl]-Peg3-Peg3, [Octadecanoyl]-[4-aminobutanoyl]-Peg3-Peg3, [Eicosanoyl]-[4-aminobutanoyl]-Peg3-Peg3,

[Dodecanoyl]-isoLys-Peg3-Peg3-Peg3, [Tetradecanoyl]-isoLys-Peg3-Peg3-Peg3, [Hexadecanoyl]-isoLys-Peg3-Peg3-Peg3, [Octadecanoyl]-isoLys-Peg3-Peg3-Peg3, [Eicosanoyl]-isoLys-Peg3-Peg3-Peg3,

[Dodecanoyl]-[4-aminobutanoyl]-Peg3-Peg3-Peg3, [Tetradecanoyl]-[4-aminobutanoyl]-Peg3-Peg3-Peg3, [Hexadecanoyl]-[4-aminobutanoyl]-Peg3-Peg3-Peg3, [Octadecanoyl]-[4-aminobutanoyl]-Peg3-Peg3-Peg3, [Eicosanoyl]-[4-aminobutanoyl]-Peg3-Peg3-Peg3,

[13-carboxy-tridecanoyl]-isoGlu, [15-carboxy-Pentadecanoyl]-isoGlu, [17-carboxy-Heptadecanoyl]-isoGlu, [19-carboxy-Nonadecanoyl]-isoGlu, [21-carboxy-heneicosanoyl]-isoGlu,

[17-carboxy-Heptadecanoyl]-βAla, [19-carboxy-Nonadecanoyl]-βAla, [21-carboxy-heneicosanoyl]-βAla, [15-carboxy-Pentadecanoyl]-βAla, [13-carboxy-tridecanoyl]-βAla,

[13-carboxy-tridecanoyl]-isoGlu-Peg3, [15-carboxy-Pentadecanoyl]-isoGlu-Peg3, [17-carboxy-Heptadecanoyl]-isoGlu-Peg3, [19-carboxy-Nonadecanoyl]-isoGlu-Peg3, [21-carboxy-heneicosanoyl]-isoGlu-Peg3,

[13-carboxy-tridecanoyl]-βAla-Peg3, [15-carboxy-Pentadecanoyl]-βAla-Peg3, [17-carboxy-Heptadecanoyl]-βAla-Peg3, [19-carboxy-Nonadecanoyl]-βAla-Peg3, [21-carboxy-heneicosanoyl]-βAla-Peg3,

[13-carboxy-tridecanoyl]-isoGlu-Peg3-Peg3, [15-carboxy-Pentadecanoyl]-isoGlu-Peg3-Peg3, [17-carboxy-Heptadecanoyl]-isoGlu-Peg3-Peg3, [19-carboxy-Nonadecanoyl]-isoGlu-Peg3-Peg3, [21-carboxy-heneicosanoyl]-isoGlu-Peg3-Peg3,

[13-carboxy-tridecanoyl]-βAla-Peg3-Peg3, [15-carboxy-Pentadecanoyl]-βAla-Peg3-Peg3, [17-carboxy-Heptadecanoyl]-βAla-Peg3-Peg3, [19-carboxy-Nonadecanoyl]-βAla-Peg3-Peg3, [21-carboxy-heneicosanoyl]-βAla-Peg3-Peg3,

[13-carboxy-tridecanoyl]-isoGlu-Peg3-Peg3-Peg3, [15-carboxy-Pentadecanoyl]-isoGlu-Peg3-Peg3-Peg3, [17-carboxy-Heptadecanoyl]-isoGlu-Peg3-Peg3-Peg3, [19-carboxy-Nonadecanoyl]-isoGlu-Peg3-Peg3-Peg3, [21-carboxy-heneicosanoyl]-isoGlu-Peg3-Peg3-Peg3,

[13-carboxy-tridecanoyl]-βAla-Peg3-Peg3-Peg3, [15-carboxy-Pentadecanoyl]-βAla-Peg3-Peg3-Peg3, [17-carboxy-Heptadecanoyl]-βAla-Peg3-Peg3-Peg3, [19-carboxy-Nonadecanoyl]-βAla-Peg3-Peg3-Peg3, [21-carboxy-heneicosanoyl]-βAla-Peg3-Peg3-Peg3,

[13-carboxy-tridecanoyl]-isoLys, [15-carboxy-Pentadecanoyl]-isoLys, [17-carboxy-Heptadecanoyl]-isoLys, [19-carboxy-Nonadecanoyl]-isoLys, [21-carboxy-heneicosanoyl]-isoLys,

[17-carboxy-Heptadecanoyl]-[4-aminobutanoyl], [19-carboxy-Nonadecanoyl]-[4-aminobutanoyl], [21-carboxy-heneicosanoyl]-[4-aminobutanoyl], [15-carboxy-Pentadecanoyl]-[4-aminobutanoyl], [13-carboxy-tridecanoyl]-[4-aminobutanoyl],

[13-carboxy-tridecanoyl]-isoLys-Peg3, [15-carboxy-Pentadecanoyl]-isoLys-Peg3, [17-carboxy-Heptadecanoyl]-isoLys-Peg3, [19-carboxy-Nonadecanoyl]-isoLys-Peg3, [21-carboxy-heneicosanoyl]-isoLys-Peg3,

[13-carboxy-tridecanoyl]-[4-aminobutanoyl]-Peg3, [15-carboxy-Pentadecanoyl]- [4-aminobutanoyl]-Peg3, [17-carboxy-Heptadecanoyl]-[4-aminobutanoyl]-Peg3,

[19-carboxy-Nonadecanoyl]-βAla-Peg3, [21-carboxy-heneicosanoyl]-βAla-Peg3,

[13-carboxy-tridecanoyl]-isoLys-Peg3-Peg3, [15-carboxy-Pentadecanoyl]-isoLys-Peg3-Peg3, [17-carboxy-Heptadecanoyl]-isoLys-Peg3-Peg3, [19-carboxy-Nonadecanoyl]-isoLys-Peg3-Peg3, [21-carboxy-heneicosanoyl]-isoLys-Peg3-Peg3,

[13-carboxy-tridecanoyl]-[4-aminobutanoyl]-Peg3-Peg3, [15-carboxy-Pentadecanoyl]-[4-aminobutanoyl]-Peg3-Peg3, [17-carboxy-Heptadecanoyl]-[4-aminobutanoyl]-Peg3-Peg3, [19-carboxy-Nonadecanoyl]-[4-aminobutanoyl]-Peg3-Peg3, [21-carboxy-heneicosanoyl]-[4-aminobutanoyl]-Peg3-Peg3,

[13-carboxy-tridecanoyl]-isoLys-Peg3-Peg3-Peg3, [15-carboxy-Pentadecanoyl]-isoLys-Peg3-Peg3-Peg3, [17-carboxy-Heptadecanoyl]-isoLys-Peg3-Peg3-Peg3, [19-carboxy-Nonadecanoyl]-isoLys-Peg3-Peg3-Peg3, [21-carboxy-heneicosanoyl]-isoLys-Peg3-Peg3-Peg3,

[13-carboxy-tridecanoyl]-[4-aminobutanoyl]-Peg3-Peg3-Peg3, [15-carboxy-Pentadecanoyl]-[4-aminobutanoyl]-Peg3-Peg3-Peg3, [17-carboxy-Heptadecanoyl]-[4-aminobutanoyl]-Peg3-Peg3-Peg3, [19-carboxy-Nonadecanoyl]-[4-aminobutanoyl]-Peg3-Peg3-Peg3 and [21-carboxy-heneicosanoyl]-[4-aminobutanoyl]-Peg3-Peg3-Peg3.

Further examples of substituents Z¹—Z²— include:

[Dodecanoyl]-isoLys, [Tetradecanoyl]-isoLys, [Hexadecanoyl]-isoLys, [Octadecanoyl]-isoLys, [Eicosanoyl]-isoLys,

[Hexadecanoyl]-[4-aminobutanoyl], [Octadecanoyl]-[4-aminobutanoyl], [Eicosanoyl]-[4-aminobutanoyl], [Tetradecanoyl]-[4-aminobutanoyl], [Dodecanoyl]-[4-aminobutanoyl],

[Hexadecanoyl]-KEK, [Octadecanoyl]- KEK, [Eicosanoyl]- KEK, [Tetradecanoyl]- KEK, [Dodecanoyl]- KEK,

[Dodecanoyl]-Peg3, [Tetradecanoyl]-Peg3, [Hexadecanoyl]-Peg3, [Octadecanoyl]-Peg3, [Eicosanoyl]-Peg3,

[Dodecanoyl]-Peg3-Peg3, [Tetradecanoyl]-Peg3-Peg3, [Hexadecanoyl]-Peg3-Peg3, [Octadecanoyl]-Peg3-Peg3, [Eicosanoyl]-Peg3-Peg3,

[Dodecanoyl]-Peg3-Peg3-Peg3, [Tetradecanoyl]-Peg3-Peg3-Peg3, [Hexadecanoyl]-Peg3-Peg3-Peg3, [Octadecanoyl]-Peg3-Peg3-Peg3, [Eicosanoyl]-Peg3-Peg3-Peg3,

[Dodecanoyl]-isoLys-Peg3, [Tetradecanoyl]-isoLys-Peg3, [Hexadecanoyl]-isoLys-Peg3, [Octadecanoyl]-isoLys-Peg3, [Eicosanoyl]-isoLys-Peg3,

[Dodecanoyl]-[4-aminobutanoyl]-Peg3, [Tetradecanoyl]- [4-aminobutanoyl]-Peg3, [Hexadecanoyl]-[4-aminobutanoyl]-Peg3, [Octadecanoyl]-[4-aminobutanoyl]-Peg3, [Eicosanoyl]-[4-aminobutanoyl]-Peg3,

[Dodecanoyl]-KEK-Peg3, [Tetradecanoyl]-KEK-Peg3, [Hexadecanoyl]-KEK-Peg3, [Octadecanoyl]-KEK-Peg3, [Eicosanoyl]-KEK-Peg3,

[Dodecanoyl]-isoLys-Peg3-Peg3, [Tetradecanoyl]-isoLys-Peg3-Peg3, [Hexadecanoyl]-isoLys-Peg3-Peg3, [Octadecanoyl]-isoLys-Peg3-Peg3, [Eicosanoyl]-isoLys-Peg3-Peg3,

[Dodecanoyl]-[4-aminobutanoyl]-Peg3-Peg3, [Tetradecanoyl]-[4-aminobutanoyl]-Peg3-Peg3, [Hexadecanoyl]-[4-aminobutanoyl]-Peg3-Peg3, [Octadecanoyl]-[4-aminobutanoyl]-Peg3-Peg3, [Eicosanoyl]-[4-aminobutanoyl]-Peg3-Peg3,

[Dodecanoyl]-KEK-Peg3-Peg3, [Tetradecanoyl]-KEK-Peg3-Peg3, [Hexadecanoyl]-KEK-Peg3-Peg3, [Octadecanoyl]- KEK -Peg3-Peg3, [Eicosanoyl]- KEK -Peg3-Peg3,

[Dodecanoyl]-isoLys-Peg3-Peg3-Peg3, [Tetradecanoyl]-isoLys-Peg3-Peg3-Peg3, [Hexadecanoyl]-isoLys-Peg3-Peg3-Peg3, [Octadecanoyl]-isoLys-Peg3-Peg3-Peg3, [Eicosanoyl]-isoLys-Peg3-Peg3-Peg3,

[Dodecanoyl]-[4-aminobutanoyl]-Peg3-Peg3-Peg3, [Tetradecanoyl]-[4-aminobutanoyl]-Peg3-Peg3-Peg3, [Hexadecanoyl]-[4-aminobutanoyl]-Peg3-Peg3-Peg3, [Octadecanoyl]-[4-aminobutanoyl]-Peg3-Peg3-Peg3, [Eicosanoyl]-[4-aminobutanoyl]-Peg3-Peg3-Peg3,

[Dodecanoyl]-KEK-Peg3-Peg3-Peg3, [Tetradecanoyl]-KEK-Peg3-Peg3-Peg3, [Hexadecanoyl]-KEK-Peg3-Peg3-Peg3, [Octadecanoyl]-KEK-Peg3-Peg3-Peg3, [Eicosanoyl]-KEK-Peg3-Peg3-Peg3,

[Dodecanoyl]-isoGlu-KEK-Peg3, [Tetradecanoyl]-isoGlu-KEK-Peg3, [Hexadecanoyl]-isoGlu-KEK-Peg3, [Octadecanoyl]-isoGlu-KEK-Peg3, [Eicosanoyl]-isoGlu-KEK-Peg3,

[Dodecanoyl]-[4-aminobutanoyl]-KEK-Peg3, [Tetradecanoyl]-[4-aminobutanoyl]-KEK-Peg3, [Hexadecanoyl]-[4-aminobutanoyl]-KEK-Peg3, [Octadecanoyl]-[4-aminobutanoyl]-KEK-Peg3, [Eicosanoyl]-[4-aminobutanoyl]-KEK-Peg3,

[Dodecanoyl]-isoLys-KEK-Peg3, [Tetradecanoyl]-isoLys-KEK-Peg3, [Hexadecanoyl]-isoLys-KEK-Peg3, [Octadecanoyl]-isoLys-KEK-Peg3, [Eicosanoyl]-isoLys-KEK-Peg3,

[Dodecanoyl]-βAla-KEK-Peg3, [Tetradecanoyl]-βAla-KEK-Peg3, [Hexadecanoyl]-βAla-KEK-Peg3, [Octadecanoyl]-βAla-KEK-Peg3, [Eicosanoyl]-βAla-KEK-Peg3,

[Dodecanoyl]-isoGlu-KEK-Peg3-Peg3, [Tetradecanoyl]-isoGlu-KEK-Peg3-Peg3, [Hexadecanoyl]-isoGlu-KEK-Peg3-Peg3, [Octadecanoyl]-isoGlu-KEK-Peg3-Peg3, [Eicosanoyl]-isoGlu-KEK-Peg3-Peg3,

[Dodecanoyl]-βAla-KEK-Peg3-Peg3, [Tetradecanoyl]-βAla-KEK-Peg3-Peg3, [Hexadecanoyl]-βAla-KEK-Peg3-Peg3, [Octadecanoyl]-βAla-KEK-Peg3-Peg3, [Eicosanoyl]-βAla-KEK-Peg3-Peg3,

[Dodecanoyl]-isoLys-KEK-Peg3-Peg3, [Tetradecanoyl]-isoLys-KEK-Peg3-Peg3, [Hexadecanoyl]-isoLys-KEK-Peg3-Peg3, [Octadecanoyl]-isoLys-KEK-Peg3-Peg3, [Eicosanoyl]-isoLys-KEK-Peg3-Peg3,

[Dodecanoyl]-[4-aminobutanoyl]-KEK-Peg3-Peg3, [Tetradecanoyl]-[4-aminobutanoyl]-KEK-Peg3-Peg3, [Hexadecanoyl]-[4-aminobutanoyl]-KEK-Peg3-Peg3, [Octadecanoyl]-[4-aminobutanoyl]-KEK-Peg3-Peg3, [Eicosanoyl]-[4-aminobutanoyl]-KEK-Peg3-Peg3,

[Dodecanoyl]-isoGlu-KEK-Peg3-Peg3-Peg3, [Tetradecanoyl]-isoGlu-KEK-Peg3-Peg3-Peg3, [Hexadecanoyl]-isoGlu-KEK-Peg3-Peg3-Peg3, [Octadecanoyl]-isoGlu-KEK-Peg3-Peg3-Peg3, [Eicosanoyl]-isoGlu-KEK-Peg3-Peg3-Peg3,

[Dodecanoyl]-βAla-KEK-Peg3-Peg3-Peg3, [Tetradecanoyl]-βAla-KEK-Peg3-Peg3-Peg3, [Hexadecanoyl]-βAla-KEK-Peg3-Peg3-Peg3, [Octadecanoyl]-βAla-KEK-Peg3-Peg3-Peg3, [Eicosanoyl]-βAla-KEK-Peg3-Peg3-Peg3,

[Dodecanoyl]-isoLys-KEK-Peg3-Peg3-Peg3, [Tetradecanoyl]-isoLys-KEK-Peg3-Peg3-Peg3, [Hexadecanoyl]-isoLys-KEK-Peg3-Peg3-Peg3, [Octadecanoyl]-isoLys-KEK-Peg3-Peg3-Peg3, [Eicosanoyl]-isoLys-KEK-Peg3-Peg3-Peg3,

[Dodecanoyl]-[4-aminobutanoyl]-KEK-Peg3-Peg3-Peg3, [Tetradecanoyl]-[4-aminobutanoyl]-KEK-Peg3-Peg3-Peg3, [Hexadecanoyl]-[4-aminobutanoyl]-KEK-Peg3-Peg3-Peg3, [Octadecanoyl]-[4-aminobutanoyl]-KEK-Peg3-Peg3-Peg3, [Eicosanoyl]-[4-aminobutanoyl]-KEK-Peg3-Peg3-Peg3,

[Dodecanoyl]-KEK-isoGlu-Peg3, [Tetradecanoyl]-KEK-isoGlu-Peg3, [Hexadecanoyl]-KEK-isoGlu-Peg3, [Octadecanoyl]-KEK-isoGlu-Peg3, [Eicosanoyl]-KEK-isoGlu-Peg3,

[Dodecanoyl]-KEK-βAla-Peg3, [Tetradecanoyl]-KEK-βAla-Peg3, [Hexadecanoyl]-KEK-βAla-Peg3, [Octadecanoyl]-KEK-βAla-Peg3, [Eicosanoyl]-KEK-βAla-Peg3,

[Dodecanoyl]-KEK-[4-aminobutanoyl]-Peg3, [Tetradecanoyl]-KEK-[4-aminobutanoyl]-Peg3, [Hexadecanoyl]-KEK-[4-aminobutanoyl]-Peg3, [Octadecanoyl]-KEK-[4-aminobutanoyl]-Peg3, [Eicosanoyl]-KEK-[4-aminobutanoyl]-Peg3,

[Dodecanoyl]-KEK-isoLys-Peg3, [Tetradecanoyl]-KEK-isoLys-Peg3, [Hexadecanoyl]-KEK-isoLys-Peg3, [Octadecanoyl]-KEK-isoLys-Peg3, [Eicosanoyl]-KEK-isoLys-Peg3,

[Dodecanoyl]-KEK-isoGlu-Peg3-Peg3, [Tetradecanoyl]-KEK-isoGlu-Peg3-Peg3, [Hexadecanoyl]-KEK-isoGlu-Peg3-Peg3, [Octadecanoyl]-KEK-isoGlu-Peg3-Peg3, [Eicosanoyl]-KEK-isoGlu-Peg3-Peg3,

[Dodecanoyl]-KEK-βAla-Peg3-Peg3, [Tetradecanoyl]-KEK-βAla-Peg3-Peg3, [Hexadecanoyl]-KEK-βAla-Peg3-Peg3, [Octadecanoyl]-KEK-βAla-Peg3-Peg3, [Eicosanoyl]-βAla-KEK-Peg3-Peg3,

[Dodecanoyl]-KEK-[4-aminobutanoyl]-Peg3-Peg3, [Tetradecanoyl]-KEK-[4-aminobutanoyl]-Peg3-Peg3, [Hexadecanoyl]-KEK-[4-aminobutanoyl]-Peg3-Peg3, [Octadecanoyl]-KEK-[4-aminobutanoyl]-Peg3-Peg3, [Eicosanoyl]-KEK-[4-aminobutanoyl]-Peg3-Peg3,

[Dodecanoyl]-KEK-isoLys-Peg3-Peg3, [Tetradecanoyl]-KEK-isoLys-Peg3-Peg3, [Hexadecanoyl]-KEK-isoLys-Peg3-Peg3, [Octadecanoyl]-KEK-isoLys-Peg3-Peg3, [Eicosanoyl]-KEK-isoLys-Peg3-Peg3,

[Dodecanoyl]-KEK-isoGlu-Peg3-Peg3-Peg3, [Tetradecanoyl]-KEK-isoGlu-Peg3-Peg3-Peg3, [Hexadecanoyl]-KEK-isoGlu-Peg3-Peg3-Peg3, [Octadecanoyl]-KEK-isoGlu-Peg3-Peg3-Peg3, [Eicosanoyl]-KEK-isoGlu-Peg3-Peg3-Peg3,

[Dodecanoyl]-KEK-βAla-Peg3-Peg3-Peg3, [Tetradecanoyl]KEK-βAla-Peg3-Peg3-Peg3, [Hexadecanoyl]-βAla-KEK-Peg3-Peg3-Peg3, [Octadecanoyl]-KEK-βAla-Peg3-Peg3-Peg3, [Eicosanoyl]-KEK-βAla-Peg3-Peg3-Peg3,

[Dodecanoyl]-KEK[4-aminobutanoyl]-Peg3-Peg3-Peg3, [Tetradecanoyl]-KEK-[4-aminobutanoyl]-Peg3-Peg3-Peg3, [Hexadecanoyl]-KEK-[4-aminobutanoyl]-Peg3-Peg3-Peg3, [Octadecanoyl]-KEK[4-aminobutanoyl]-Peg3-Peg3-Peg3, [Eicosanoyl]-KEK-[4-aminobutanoyl]-Peg3-Peg3-Peg3,

[Dodecanoyl]-KEK-isoLys-Peg3-Peg3-Peg3, [Tetradecanoyl]-KEK-isoLys-Peg3-Peg3-Peg3, [Hexadecanoyl]-KEK-isoLys-Peg3-Peg3-Peg3, [Octadecanoyl]-KEK-isoLys-Peg3-Peg3-Peg3, [Eicosanoyl]-KEK-isoLys-Peg3-Peg3-Peg3,

[13-carboxy-tridecanoyl]-isoGlu, [15-carboxy-Pentadecanoyl]-isoGlu, [17-carboxy-Heptadecanoyl]-isoGl u, [19-carboxy-Nonadecanoyl]-isoGlu, [21-carboxy-hen21-carboxy-heneicosanoyl]-isoGlu,

[17-carboxy-Heptadecanoyl]-βAla, [19-carboxy-Nonadecanoyl]-βAla, [21-carboxy-heneicosanoyl]-βAla, [15-carboxy-Pentadecanoyl]-βAla, [13-carboxy-tridecanoyl]-βAla,

[13-carboxy-tridecanoyl]-isoLys, [15-carboxy-Pentadecanoyl]-isoLys, [17-carboxy-Heptadecanoyl]-isoLys, [19-carboxy-Nonadecanoyl]-isoLys, [21-carboxy-heneicosanoyl]-isoLys,

[17-carboxy-Heptadecanoyl]-[4-aminobutanoyl], [19-carboxy-Nonadecanoyl]-[4-aminobutanoyl], [21-carboxy-heneicosanoyl]-[4-aminobutanoyl], [15-carboxy-Pentadecanoyl]-[4-aminobutanoyl], [13-carboxy-tridecanoyl]-[4-aminobutanoyl],

[17-carboxy-Heptadecanoyl]-KEK, [19-carboxy-Nonadecanoyl]- KEK, [21-carboxy-heneicosanoyl]- KEK, [15-carboxy-Pentadecanoyl]- KEK, [13-carboxy-tridecanoyl]-KEK,

[13-carboxy-tridecanoyl]-Peg3, [15-carboxy-Pentadecanoyl]-Peg3, [17-carboxy-Heptadecanoyl]-Peg3, [19-carboxy-Nonadecanoyl]-Peg3, [21-carboxy-heneicosanoyl]-Peg3,

[13-carboxy-tridecanoyl]-Peg3-Peg3, [15-carboxy-Pentadecanoyl]-Peg3-Peg3,

[17-carboxy-Heptadecanoyl]-Peg3-Peg3, [19-carboxy-Nonadecanoyl]-Peg3-Peg3, [21-carboxy-heneicosanoyl]-Peg3-Peg3,

[13-carboxy-tridecanoyl]-Peg3-Peg3-Peg3, [15-carboxy-Pentadecanoyl]-Peg3-Peg3-Peg3, [17-carboxy-Heptadecanoyl]-Peg3-Peg3-Peg3, [19-carboxy-Nonadecanoyl]-Peg3-Peg3-Peg3, [21-carboxy-heneicosanoyl]-Peg3-Peg3-Peg3,

[13-carboxy-tridecanoyl]-isoGlu-Peg3, [15-carboxy-Pentadecanoyl]-isoGlu-Peg3, [17-carboxy-Heptadecanoyl]-isoGlu-Peg3, [19-carboxy-Nonadecanoyl]-isoGlu-Peg3, [21-carboxy-heneicosanoyl]-isoGlu-Peg3,

[13-carboxy-tridecanoyl]-βAla-Peg3, [15-carboxy-Pentadecanoyl]-βAla-Peg3, [17-carboxy-Heptadecanoyl]-βAla-Peg3, [19-carboxy-Nonadecanoyl]-βAla-Peg3, [21-carboxy-heneicosanoyl]-βAla-Peg3,

[13-carboxy-tridecanoyl]-isoLys-Peg3, [15-carboxy-Pentadecanoyl]-isoLys-Peg3, [17-carboxy-Heptadecanoyl]-isoLys-Peg3, [19-carboxy-Nonadecanoyl]-isoLys-Peg3, [21-carboxy-heneicosanoyl]-isoLys-Peg3,

[13-carboxy-tridecanoyl]-[4-aminobutanoyl]-Peg3, [15-carboxy-Pentadecanoyl]- [4-aminobutanoyl]-Peg3, [17-carboxy-Heptadecanoyl]-[4-aminobutanoyl]-Peg3, [19-carboxy-Nonadecanoyl]-[4-aminobutanoyl]-Peg3, [21-carboxy-heneicosanoyl]-[4-aminobutanoyl]-Peg3,

[13-carboxy-tridecanoyl]-KEK-Peg3, [15-carboxy-Pentadecanoyl]-KEK-Peg3, [17-carboxy-Heptadecanoyl]-KEK-Peg3, [19-carboxy-Nonadecanoyl]-KEK-Peg3, [21-carboxy-heneicosanoyl]-KEK-Peg3,

[13-carboxy-tridecanoyl]-isoGlu-Peg3-Peg3, [15-carboxy-Pentadecanoyl]-isoGlu-Peg3-Peg3, [17-carboxy-Heptadecanoyl]-isoGlu-Peg3-Peg3, [19-carboxy-Nonadecanoyl]-isoGlu-Peg3-Peg3, [21-carboxy-heneicosanoyl]-isoGlu-Peg3-Peg3,

[13-carboxy-tridecanoyl]-βAla-Peg3-Peg3, [15-carboxy-Pentadecanoyl]-βAla-Peg3-Peg3, [17-carboxy-Heptadecanoyl]-βAla-Peg3-Peg3, [19-carboxy-Nonadecanoyl]-βAla-Peg3-Peg3, [21-carboxy-heneicosanoyl]-βAla-Peg3-Peg3,

[13-carboxy-tridecanoyl]-isoLys-Peg3-Peg3, [15-carboxy-Pentadecanoyl]-isoLys-Peg3-Peg3, [17-carboxy-Heptadecanoyl]-isoLys-Peg3-Peg3, [19-carboxy-Nonadecanoyl]-isoLys-Peg3-Peg3, [21-carboxy-heneicosanoyl]-isoLys-Peg3-Peg3,

[13-carboxy-tridecanoyl]-[4-aminobutanoyl]-Peg3-Peg3, [15-carboxy-Pentadecanoyl]-[4-aminobutanoyl]-Peg3-Peg3, [17-carboxy-Heptadecanoyl]-[4-aminobutanoyl]-Peg3-Peg3, [19-carboxy-Nonadecanoyl]-[4-aminobutanoyl]-Peg3-Peg3, [21-carboxy-heneicosanoyl]-[4-aminobutanoyl]-Peg3-Peg3,

[13-carboxy-tridecanoyl]-KEK-Peg3-Peg3, [15-carboxy-Pentadecanoyl]-KEK-Peg3-Peg3, [17-carboxy-Heptadecanoyl]- KEK-Peg3-Peg3, [19-carboxy-Nonadecanoyl]-KEK -Peg3-Peg3, [21-carboxy-heneicosanoyl]- KEK -Peg3-Peg3,

[13-carboxy-tridecanoyl]-isoGlu-Peg3-Peg3-Peg3, [15-carboxy-Pentadecanoyl]-isoGlu-Peg3-Peg3-Peg3, [17-carboxy-Heptadecanoyl]-isoGlu-Peg3-Peg3-Peg3, [19-carboxy-Nonadecanoyl]-isoGlu-Peg3-Peg3-Peg3, [21-carboxy-heneicosanoyl]-isoGlu-Peg3-Peg3-Peg3,

[13-carboxy-tridecanoyl]-βAla-Peg3-Peg3-Peg3, [15-carboxy-Pentadecanoyl]-βAla-Peg3-Peg3-Peg3, [17-carboxy-Heptadecanoyl]-βAla-Peg3-Peg3-Peg3, [19-carboxy-Nonadecanoyl]-βAla-Peg3-Peg3-Peg3, [21-carboxy-heneicosanoyl]-βAla-Peg3-Peg3-Peg3,

[13-carboxy-tridecanoyl]-isoLys-Peg3-Peg3-Peg3, [15-carboxy-Pentadecanoyl]-isoLys-Peg3-Peg3-Peg3, [17-carboxy-Heptadecanoyl]-isoLys-Peg3-Peg3-Peg3, [19-carboxy-Nonadecanoyl]-isoLys-Peg3-Peg3-Peg3, [21-carboxy-heneicosanoyl]-isoLys-Peg3-Peg3-Peg3,

[13-carboxy-tridecanoyl]-[4-aminobutanoyl]-Peg3-Peg3-Peg3, [15-carboxy-Pentadecanoyl]-[4-aminobutanoyl]-Peg3-Peg3-Peg3, [17-carboxy-Heptadecanoyl]-[4-aminobutanoyl]-Peg3-Peg3-Peg3, [19-carboxy-Nonadecanoyl]-[4-aminobutanoyl]-Peg3-Peg3-Peg3, [21-carboxy-heneicosanoyl]-[4-aminobutanoyl]-Peg3-Peg3-Peg3,

[13-carboxy-tridecanoyl]- KEK-Peg3-Peg3-Peg3, [15-carboxy-Pentadecanoyl]-KEK-Peg3-Peg3-Peg3, [17-carboxy-Heptadecanoyl]-KEK-Peg3-Peg3-Peg3, [19-carboxy-Nonadecanoyl]-KEK-Peg3-Peg3-Peg3, [21-carboxy-heneicosanoyl]-KEK-Peg3-Peg3-Peg3,

[13-carboxy-tridecanoyl]-isoGlu-KEK-Peg3, [15-carboxy-Pentadecanoyl]-isoGlu-KEK-Peg3, [17-carboxy-Heptadecanoyl]-isoGlu-KEK-Peg3, [19-carboxy-Nonadecanoyl]-isoGlu-KEK-Peg3, [21-carboxy-heneicosanoyl]-isoGlu-KEK-Peg3,

[13-carboxy-tridecanoyl]-[4-arninobutanoyl]-KEK-Peg3, [15-carboxy-Pentadecanoyl]-[4-aminobutanoyl]-KEK-Peg3, [17-carboxy-Heptadecanoyl]-[4-aminobutanoyl]-KEK-Peg3, [19-carboxy-Nonadecanoyl]-[4-aminobutanoyl]-KEK-Peg3, [21-carboxy-heneicosanoyl]-[4-aminobutanoyl]-KEK-Peg3,

[13-carboxy-tridecanoyl]-isoLys-KEK-Peg3, [15-carboxy-Pentadecanoyl]-isoLys-KEK-Peg3, [17-carboxy-Heptadecanoyl]-isoLys-KEK-Peg3, [19-carboxy-Nonadecanoyl]-isoLys-KEK-Peg3, [21-carboxy-heneicosanoyl]-isoLys-KEK-Peg3,

[13-carboxy-tridecanoyl]-βAla-KEK-Peg3, [15-carboxy-Pentadecanoyl]-βAla-KEK-Peg3, [17-carboxy-Heptadecanoyl]-βAla-KEK-Peg3, [19-carboxy-Nonadecanoyl]-βAla-KEK-Peg3, [21-carboxy-heneicosanoyl]-βAla-KEK-Peg3,

[13-carboxy-tridecanoyl]-isoGlu-KEK-Peg3-Peg3, [15-carboxy-Pentadecanoyl]-isoGlu-KEK-Peg3-Peg3, [17-carboxy-Heptadecanoyl]-isoGlu-KEK-Peg3-Peg3, [19-carboxy-Nonadecanoyl]-isoGlu-KEK-Peg3-Peg3, [21-carboxy-heneicosanoyl]-isoGlu-KEK-Peg3-Peg3,

[13-carboxy-tridecanoyl]-βAla-KEK-Peg3-Peg3, [15-carboxy-Pentadecanoyl]-βAla-KEK-Peg3-Peg3, [17-carboxy-Heptadecanoyl]-βAla-KEK-Peg3-Peg3, [19-carboxy-Nonadecanoyl]-βAla-KEK-Peg3-Peg3, [21-carboxy-heneicosanoyl]-βAla-KEK-Peg3-Peg3,

[13-carboxy-tridecanoyl]-isoLys-KEK-Peg3-Peg3, [15-carboxy-Pentadecanoyl]-isoLys-KEK-Peg3-Peg3, [17-carboxy-Heptadecanoyl]-isoLys-KEK-Peg3-Peg3, [19-carboxy-Nonadecanoyl]-isoLys-KEK-Peg3-Peg3, [21-carboxy-heneicosanoyl]-isoLys-KEK-Peg3-Peg3,

[13-carboxy-tridecanoyl]-[4-aminobutanoyl]-KEK-Peg3-Peg3, [15-carboxy-Pentadecanoyl]-[4-aminobutanoyl]-KEK-Peg3-Peg3, [17-carboxy-Heptadecanoyl]-[4-aminobutanoyl]-KEK-Peg3-Peg3, [19-carboxy-Nonadecanoyl]-[4-aminobutanoyl]-KEK-Peg3-Peg3, [21-carboxy-heneicosanoyl]-[4-aminobutanoyl]-KEK-Peg3-Peg3,

[13-carboxy-tridecanoyl]-isoGlu-KEK-Peg3-Peg3-Peg3, [15-carboxy-Pentadecanoyl]-isoGlu-KEK-Peg3-Peg3-Peg3, [17-carboxy-Heptadecanoyl]-isoGlu-KEK-Peg3-Peg3-Peg3, [19-carboxy-Nonadecanoyl]-isoGlu-KEK-Peg3-Peg3-Peg3, [21-carboxy-heneicosanoyl]-isoGlu-KEK-Peg3-Peg3-Peg3,

[13-carboxy-tridecanoyl]-βAla-KEK-Peg3-Peg3-Peg3, [15-carboxy-Pentadecanoyl]-βAla-KEK-Peg3-Peg3-Peg3, [17-carboxy-Heptadecanoyl]-βAla-KEK-Peg3-Peg3-Peg3, [19-carboxy-Nonadecanoyl]-βAla-KEK-Peg3-Peg3-Peg3, [21-carboxy-heneicosanoyl]-βAla-KEK-Peg3-Peg3-Peg3,

[13-carboxy-tridecanoyl]-isoLys-KEK-Peg3-Peg3-Peg3, [15-carboxy-Pentadecanoyl]-isoLys-KEK-Peg3-Peg3-Peg3, [17-carboxy-Heptadecanoyl]-isoLys-KEK-Peg3-Peg3-Peg3, [19-carboxy-Nonadecanoyl]-isoLys-KEK-Peg3-Peg3-Peg3, [21-carboxy-heneicosanoyl]-isoLys-KEK-Peg3-Peg3-Peg3,

[13-carboxy-tridecanoyl]-[4-aminobutanoyl]-KEK-Peg3-Peg3-Peg3, [15-carboxy-Pentadecanoyl]-[4-aminobutanoyl]-KEK-Peg3-Peg3-Peg3, [17-carboxy-Heptadecanoyl]-[4-aminobutanoyl]-KEK-Peg3-Peg3-Peg3, [19-carboxy-Nonadecanoyl]-[4-aminobutanoyl]-KEK-Peg3-Peg3-Peg3, [21-carboxy-heneicosanoyl]-[4-aminobutanoyl]-KEK-Peg3-Peg3-Peg3,

[13-carboxy-tridecanoyl]-KEK-isoGlu-Peg3, [15-carboxy-Pentadecanoyl]-KEK-isoGlu-Peg3, [17-carboxy-Heptadecanoyl]-KEK-isoGlu-Peg3, [19-carboxy-Nonadecanoyl]-KEK-isoGlu-Peg3, [21-carboxy-heneicosanoyl]-KEK-isoGlu-Peg3,

[13-carboxy-tridecanoyl]-KEK-βAla-Peg3, [15-carboxy-Pentadecanoyl]-KEK-βAla-Peg3, [17-carboxy-Heptadecanoyl]-KEK-βAla-Peg3, [19-carboxy-Nonadecanoyl]-KEK-βAla-Peg3, [21-carboxy-heneicosanoyl]-KEK-βAla-Peg3,

[13-carboxy-tridecanoyl]-KEK[4-aminobutanoyl]-Peg3, [15-carboxy-Pentadecanoyl]-KEK-[4-aminobutanoyl]-Peg3, [17-carboxy-Heptadecanoyl]-KEK-[4-aminobutanoyl]-Peg3, [19-carboxy-Nonadecanoyl]-KEK[4-aminobutanoyl]-Peg3, [21-carboxy-heneicosanoyl]-KEK-[4-aminobutanoyl]-Peg3,

[13-carboxy-tridecanoyl]-KEK-isoLys-Peg3, [15-carboxy-Pentadecanoyl]-KEK-isoLys-Peg3, [17-carboxy-Heptadecanoyl]-KEK-isoLys-Peg3, [19-carboxy-Nonadecanoyl]-KEK-isoLys-Peg3, [21-carboxy-heneicosanoyl]-KEK-isoLys-Peg3,

[13-carboxy-tridecanoyl]-KEK-isoGlu-Peg3-Peg3, [15-carboxy-Pentadecanoyl]-KEK-isoGlu-Peg3-Peg3, [17-carboxy-Heptadecanoyl]-KEK-isoGlu-Peg3-Peg3, [19-carboxy-Nonadecanoyl]-KEK-isoGlu-Peg3-Peg3, [21-carboxy-heneicosanoyl]-KEK-isoGlu-Peg3-Peg3,

[13-carboxy-tridecanoyl]-KEK-βAla-Peg3-Peg3, [15-carboxy-Pentadecanoyl]-KEK-βAla-Peg3-Peg3, [17-carboxy-Heptadecanoyl]-KEK-βAla-Peg3-Peg3, [19-carboxy-Nonadecanoyl]-KEK-βAla-Peg3-Peg3, [21-carboxy-heneicosanoyl]-βAla-KEK-Peg3-Peg3,

[13-carboxy-tridecanoyl]-KEK[4-aminobutanoyl]-Peg3-Peg3, [15-carboxy-Pentadecanoyl]-KEK-[4-aminobutanoyl]-Peg3-Peg3, [17-carboxy-Heptadecanoyl]-KEK-[4-aminobutanoyl]-Peg3-Peg3, [19-carboxy-Nonadecanoyl]-KEK-[4-aminobutanoyl]-Peg3-Peg3, [21-carboxy-heneicosanoyl]-KEK-[4-aminobutanoyl]-Peg3-Peg3,

[13-carboxy-tridecanoyl]-KEK-isoLys-Peg3-Peg3, [15-carboxy-Pentadecanoyl]-KEK-isoLys-Peg3-Peg3, [17-carboxy-Heptadecanoyl]-KEK-isoLys-Peg3-Peg3, [19-carboxy-Nonadecanoyl]-KEK-isoLys-Peg3-Peg3, [21-carboxy-heneicosanoyl]-KEK-isoLys-Peg3-Peg3,

[13-carboxy-tridecanoyl]-KEK-isoGlu-Peg3-Peg3-Peg3, [15-carboxy-Pentadecanoyl]-KEK-isoGlu-Peg3-Peg3-Peg3, [17-carboxy-Heptadecanoyl]-KEK-isoGlu-Peg3-Peg3-Peg3, [19-carboxy-Nonadecanoyl]-KEK-isoGlu-Peg3-Peg3-Peg3, [21-carboxy-heneicosanoyl]-KEK-isoGlu-Peg3-Peg3-Peg3,

[13-carboxy-tridecanoyl]-KEK-βAla-Peg3-Peg3-Peg3, [15-carboxy-Pentadecanoyl]KEK-βAla-Peg3-Peg3-Peg3, [17-carboxy-Heptadecanoyl]-βAla-KEK-Peg3-Peg3-Peg3, [19-carboxy-Nonadecanoyl]-KEK-βAla-Peg3-Peg3-Peg3, [21-carboxy-heneicosanoyl]-KEK-βAla-Peg3-Peg3-Peg3,

[13-carboxy-tridecanoyl]-KEK[4-aminobutanoyl]-Peg3-Peg3-Peg3, [15-carboxy-Pentadecanoyl]-KEK-[4-aminobutanoyl]-Peg3-Peg3-Peg3, [17-carboxy-Heptadecanoyl]-KEK-[4-aminobutanoyl]-Peg3-Peg3-Peg3, [19-carboxy-Nonadecanoyl]-KEK-[4-aminobutanoyl]-Peg3-Peg3-Peg3, [21-carboxy-heneicosanoyl]-KEK-[4-aminobutanoyl]-Peg3-Peg3-Peg3,

[13-carboxy-tridecanoyl]-KEK-isoLys-Peg3-Peg3-Peg3, [15-carboxy-Pentadecanoyl]-KEK-isoLys-Peg3-Peg3-Peg3, [17-carboxy-Heptadecanoyl]-KEK-isoLys-Peg3-Peg3-Peg3, [19-carboxy-Nonadecanoyl]-KEK-isoLys-Peg3-Peg3-Peg3, [21-carboxy-heneicosanoyl]-KEK-isoLys-Peg3-Peg3-Peg3.

Certain preferred substituents Z¹— and Z¹—Z²— include:

[Hexadecanoyl], [Octadecanoyl], [17-Carboxy-heptadecanoyl], [19-Carboxy-nonadecanoyl],

[Hexadecanoyl]-isoGlu, [Octadecanoyl]-isoGlu,

[Hexadecanoyl]-βAla, [Octadecanoyl]-βAla,

[Hexadecanoyl]-isoGlu-Peg3,

[Hexadecanoyl]-βAla-Peg3,

[Hexadecanoyl]-isoGlu-Peg3-Peg3,

[Hexadecanoyl]-βAla-Peg3-Peg3,

[Hexadecanoyl]-βAla-Peg3-Peg3-Peg3,

[Hexadecanoyl]-isoLys,

[Hexadecanoyl]-[4-aminobutanoyl],

[Hexadecanoyl]-isoLys-Peg3,

[Hexadecanoyl]-[4-aminobutanoyl]-Peg3,

[Hexadecanoyl]-isoLys-Peg3-Peg3,

[Hexadecanoyl]-[4-aminobutanoyl]-Peg3-Peg3,

[Hexadecanoyl]-isoLys-Peg3-Peg3-Peg3,

[Hexadecanoyl]-[4-aminobutanoyl]-Peg3-Peg3-Peg3,

[17-carboxy-Heptadecanoyl]-isoGlu,

[19-carboxy-Nonadecanoyl]-isoGlu,

[17-carboxy-Heptadecanoyl]-βAla,

[19-carboxy-Nonadecanoyl]-βAla,

[17-carboxy-Heptadecanoyl]-isoGlu-Peg3,

[19-carboxy-Nonadecanoyl]-isoGlu-Peg3,

[17-carboxy-Heptadecanoyl]-βAla-Peg3,

[19-carboxy-Nonadecanoyl]-βAla-Peg3,

[17-carboxy-Heptadecanoyl]-isoGlu-Peg3-Peg3,

[19-carboxy-Nonadecanoyl]-isoGlu-Peg3-Peg3,

[17-carboxy-Heptadecanoyl]-βAla-Peg3-Peg3,

[19-carboxy-Nonadecanoyl]-βAla-Peg3-Peg3,

[17-carboxy-Heptadecanoyl]-isoGlu-Peg3-Peg3-Peg3,

[19-carboxy-Nonadecanoyl]-isoGlu-Peg3-Peg3-Peg3,

[17-carboxy-Heptadecanoyl]-βAla-Peg3-Peg3-Peg3,

[19-carboxy-Nonadecanoyl]-βAla-Peg3-Peg3-Peg3,

[17-carboxy-Heptadecanoyl]-isoLys,

[19-carboxy-Nonadecanoyl]-isoLys,

[17-carboxy-Heptadecanoyl]-[4-aminobutanoyl],

[19-carboxy-Nonadecanoyl]-[4-aminobutanoyl],

[17-carboxy-Heptadecanoyl]-isoLys-Peg3,

[19-carboxy-Nonadecanoyl]-isoLys-Peg3,

[17-carboxy-Heptadecanoyl]-[4-aminobutanoyl]-Peg3,

[19-carboxy-Nonadecanoyl]-[4-aminobutanoyl]-Peg3,

[17-carboxy-Heptadecanoyl]-isoLys-Peg3-Peg3,

[19-carboxy-Nonadecanoyl]-isoLys-Peg3-Peg3,

[17-carboxy-Heptadecanoyl]-[4-arninobutanoyl]-Peg3-Peg3,

[19-carboxy-Nonadecanoyl]-[4-aminobutanoyl]-Peg3-Peg3,

[17-carboxy-Heptadecanoyl]-isoLys-Peg3-Peg3-Peg3,

[19-carboxy-Nonadecanoyl]-isoLys-Peg3-Peg3-Peg3,

[17-carboxy-Heptadecanoyl]-[4-arninobutanoyl]-Peg3-Peg3-Peg3,

[19-carboxy-Nonadecanoyl]-[4-aminobutanoyl]-Peg3-Peg3-Peg3.

More preferred substituents Z¹—Z²— include:

[Hexadecanoyl]-isoGlu,

[Hexadecanoyl]-βAla,

[Hexadecanoyl]-isoGlu-Peg3,

[Hexadecanoyl]-βAla-Peg3,

[Hexadecanoyl]-isoGlu-Peg3-Peg3,

[Hexadecanoyl]-isoLys,

[Hexadecanoyl]-isoLys-Peg3,

[Hexadecanoyl]-isoLys-Peg3-Peg3,

[17-carboxy-Heptadecanoyl]-isoGlu,

[19-carboxy-Nonadecanoyl]-isoGlu,

[17-carboxy-Heptadecanoyl]-isoGlu-Peg3,

[19-carboxy-Nonadecanoyl]-isoGlu-Peg3,

[17-carboxy-Heptadecanoyl]-isoGlu-Peg3-Peg3,

[19-carboxy-Nonadecanoyl]-isoGlu-Peg3-Peg3,

[17-carboxy-Heptadecanoyl]-isoGlu-Peg3-Peg3-Peg3,

[19-carboxy-Nonadecanoyl]-isoGlu-Peg3-Peg3-Peg3,

[17-carboxy-Heptadecanoyl]-isoLys,

[19-carboxy-Nonadecanoyl]-isoLys,

[17-carboxy-Heptadecanoyl]-isoLys-Peg3,

[19-carboxy-Nonadecanoyl]-isoLys-Peg3,

[17-carboxy-Heptadecanoyl]-isoLys-Peg3-Peg3,

[19-carboxy-Nonadecanoyl]-isoLys-Peg3-Peg3,

[17-carboxy-Heptadecanoyl]-isoLys-Peg3-Peg3-Peg3,

[19-carboxy-Nonadecanoyl]-isoLys-Peg3-Peg3-Peg3.

Yet further preferred substituents Z¹—Z²— include:

[Hexadecanoyl]-KEK, [Octadecanoyl]-KEK,

[Hexadecanoyl]-βAla-Peg3,

[Hexadecanoyl]-KEK-Peg3,

[Hexadecanoyl]-KEK-Peg3-Peg3,

[Hexadecanoyl]-KEK-Peg3-Peg3-Peg3,

[17-carboxy-Heptadecanoyl]-KEK,

[19-carboxy-Nonadecanoyl]-KEK,

[17-carboxy-Heptadecanoyl]-KEK-Peg3,

[19-carboxy-Nonadecanoyl]-KEK-Peg3,

[17-carboxy-Heptadecanoyl]-KEK-Peg3-Peg3,

[19-carboxy-Nonadecanoyl]-KEK-Peg3-Peg3,

[17-carboxy-Heptadecanoyl]-isoGlu-KEK

[19-carboxy-Nonadecanoyl]-isoGlu-KEK,

[17-carboxy-Heptadecanoyl]-isoLys-KEK

[19-carboxy-Nonadecanoyl]-isoLys-KEK,

[17-carboxy-Heptadecanoyl]-βAla-KEK

[19-carboxy-Nonadecanoyl]-βAla-KEK, [17-carboxy-Heptadecanoyl]-KEK-Peg3-Peg3-Peg3,

[19-carboxy-Nonadecanoyl]-KEK-Peg3-Peg3-Peg3,

[17-carboxy-Heptadecanoyl]-[4-aminobutanoyl]-KEK,

[19-carboxy-Nonadecanoyl]-[4-aminobutanoyl]-KEK,

[17-carboxy-Heptadecanoyl]-[4-aminobutanoyl]-KEK-Peg3-Peg3-Peg3,

[19-carboxy-Nonadecanoyl]-[4-aminobutanoyl]-KEK-Peg3-Peg3-Peg3,

[17-carboxy-Heptadecanoyl]-[4-aminobutanoyl]-KEK-Peg3-Peg3,

[19-carboxy-Nonadecanoyl]-[4-aminobutanoyl]-KEK-Peg3-Peg3,

[Hexadecanoyl]-isoGlu-KEK-Peg3,

[Hexadecanoyl]-isoGlu-KEK-Peg3-Peg3,

[19-carboxy-Nonadecanoyl]-isoGlu-KEK,

[19-carboxy-Nonadecanoyl]-[4-aminobutanoyl]-KEK,

[17-carboxy-Heptadecanoyl]-[4-aminobutanoyl]-KEK-Peg3-Peg3-Peg3,

[17-carboxy-Heptadecanoyl]-[4-aminobutanoyl]-KEK-Peg3-Peg3,

[19-carboxy-Nonadecanoyl]-[4-aminobutanoyl]-KEK-Peg3-Peg3,

[17-carboxy-Heptadecanoyl]-KEK-Peg3-Peg3,

[19-carboxy-Nonadecanoyl]-KEK-Peg3-Peg3,

[17-carboxy-Heptadecanoyl]-isoGlu-KEK-Peg3,

[19-carboxy-Nonadecanoyl]-isoGlu-KEK-Peg3,

[17-carboxy-Heptadecanoyl]-isoGlu-KEK-Peg3-Peg3,

[19-carboxy-Nonadecanoyl]-isoGlu-KEK-Peg3-Peg3.

Examples of Ψ comprising different substituents (fatty acids, FA), conjugated to the amino acid side-chain, optionally by a spacer, are illustrated below:

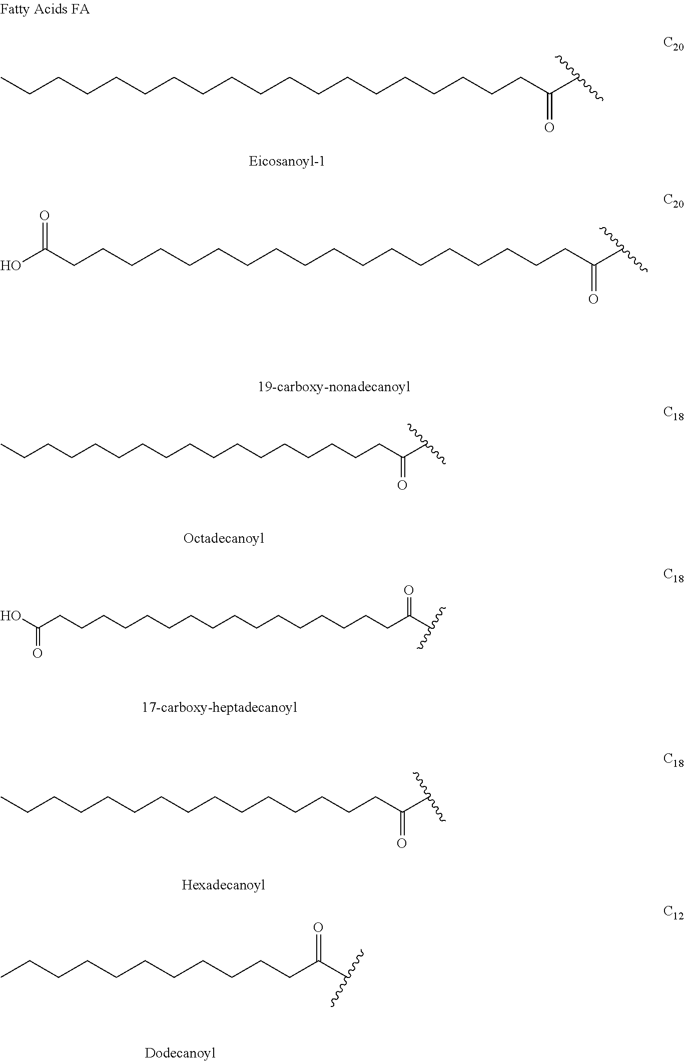 [SMILES: *C(=O)CCCCCCCCCCCCCCCCCCC ; *C(=O)CCCCCCCCCCCCCCCCCCC(=O)O ; *C(=O)CCCCCCCCCCCCCCCCC ; *C(=O)CCCCCCCCCCCCCCCCC(=O)O ; *C(=O)CCCCCCCCCCCCCCC ; *C(=O)CCCCCCCCCCC]

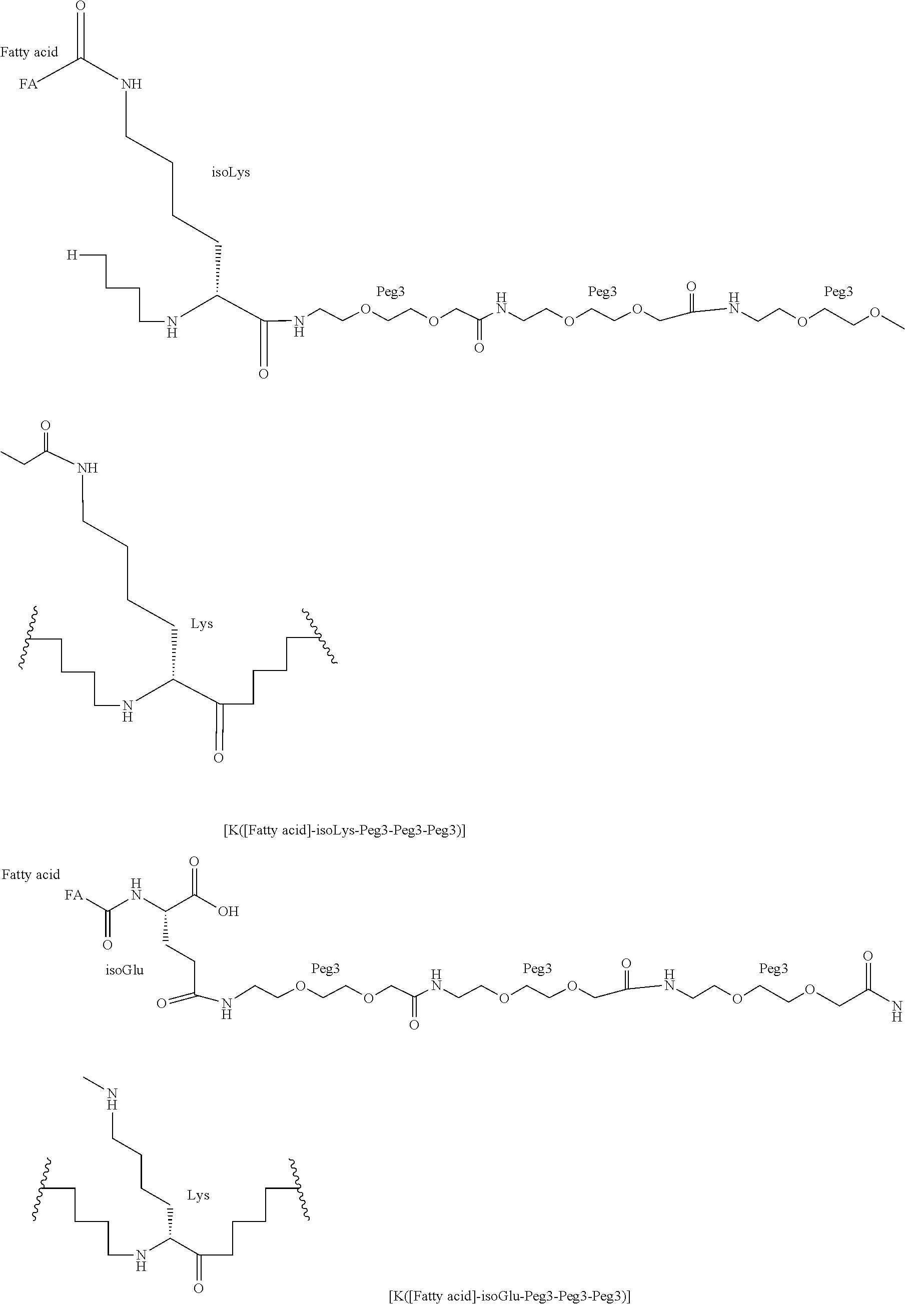

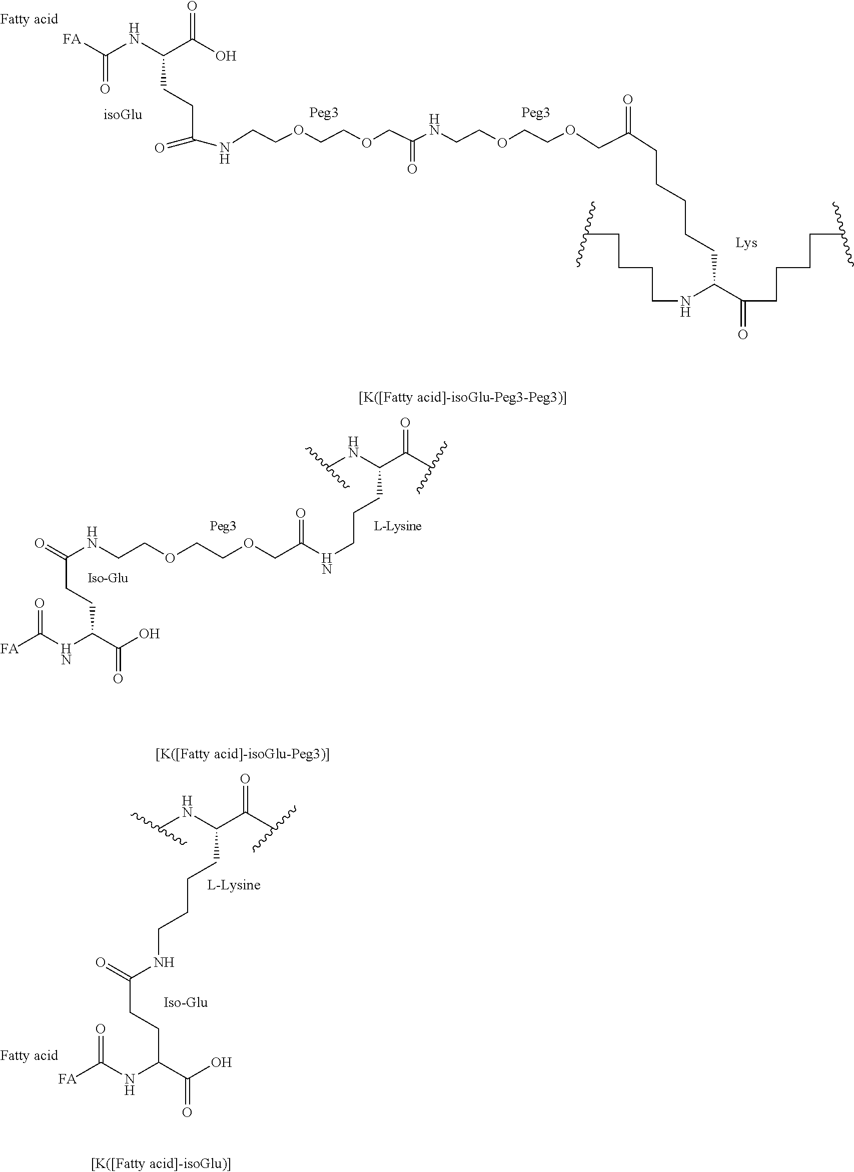

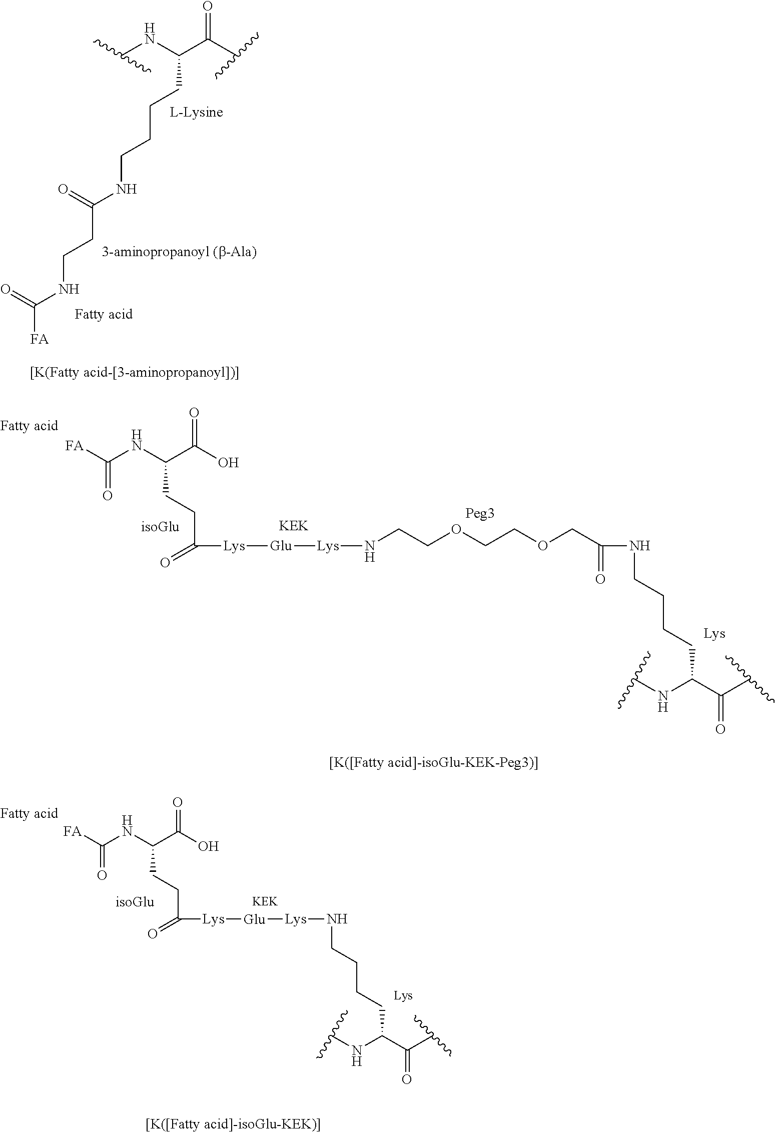

Furthermore, the substituent [Hexadecanoyl]-isoGlu, conjugated to the side chain of a lysine residue, is illustrated below:

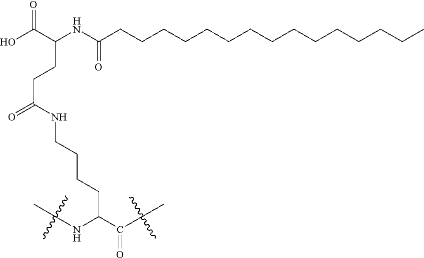 [SMILES: *NC(CCCCNC(=O)CCC(NC(=O)CCCCCCCCCCCCCCC)C(=O)O)C(*)=O]

Thus, the side chain of the Lys residue is covalently attached to the side-chain carboxyl group of the isoGlu spacer —Z2- (—Z^{S1}—) via an amide linkage. A hexadecanoyl group (Z¹) is covalently attached to the amino group of the isoGlu spacer via an amide linkage.

The substituent [Hexadecanoyl]-[4-aminobutanoyl]- conjugated to the side chain of a lysine residue, is illustrated below

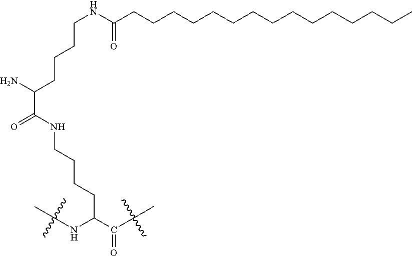 [SMILES: *NC(CCCCNC(=O)C(N)CCCCNC(=O)CCCCCCCCCCCCCCC)C(*)=O]

The substituent [(Hexadecanoyl)iso-Lys]- conjugated to the side chain of a lysine residue, is illustrated below

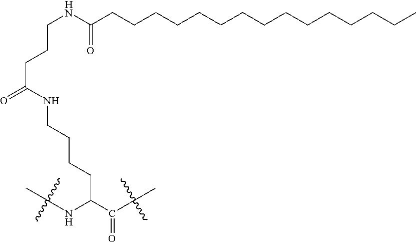 [SMILES: *NC(CCCCNC(=O)CCCNC(=O)CCCCCCCCCCCCCCC)C(*)=O]

The substituent [(Hexadecanoyl)β-Ala]- conjugated to the side chain of a lysine residue, is illustrated below

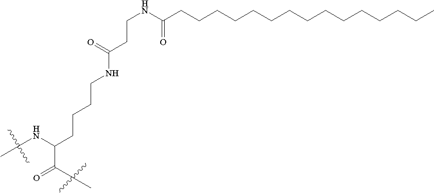 [SMILES: *NC(CCCCNC(=O)CCNC(=O)CCCCCCCCCCCCCCC)C(*)=O]

Some further specific examples of —Z²—Z¹ combinations are illustrated below. In each case, - - - indicates the point of attachment to the side chain of the amino acid component of Ψ:

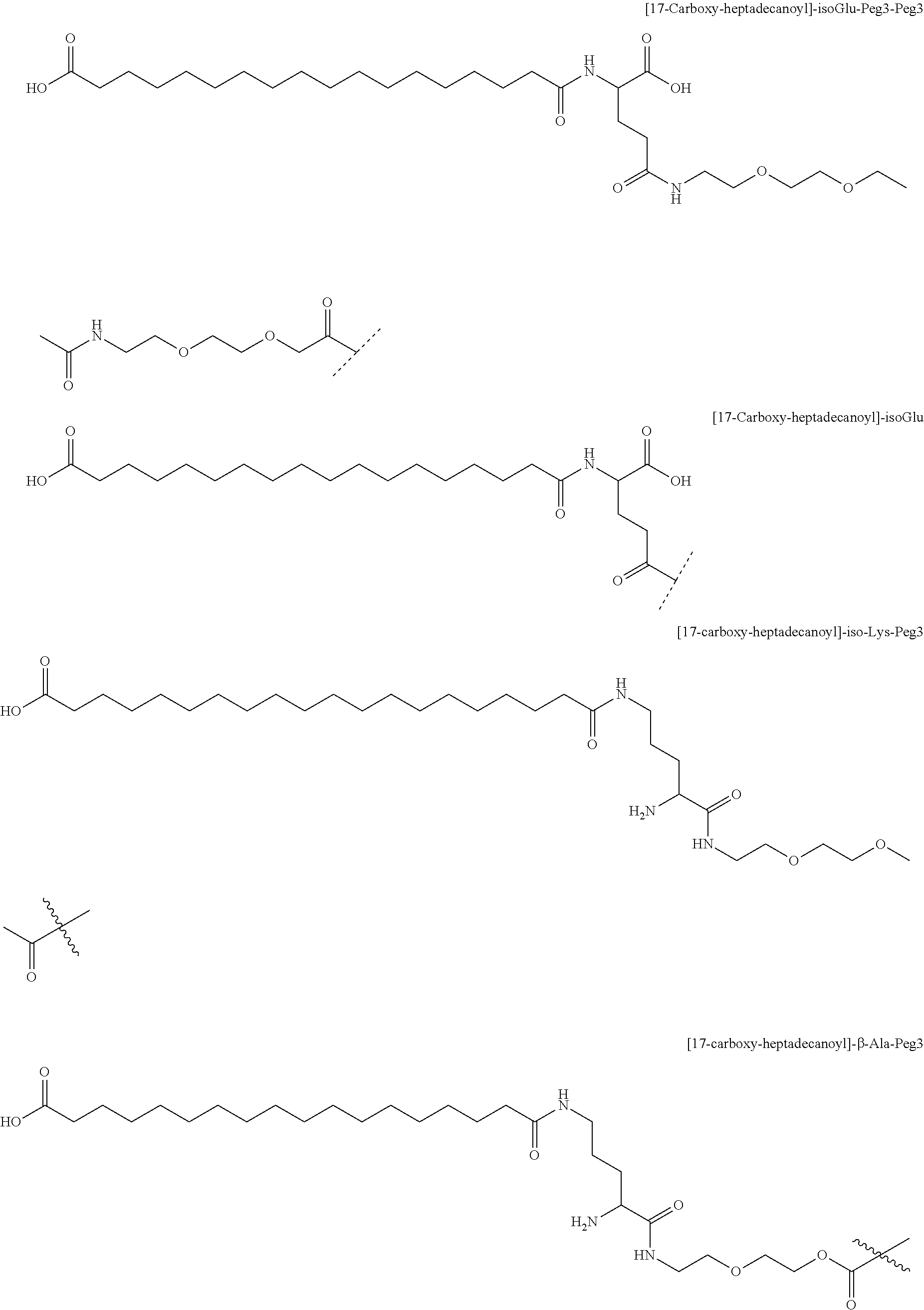

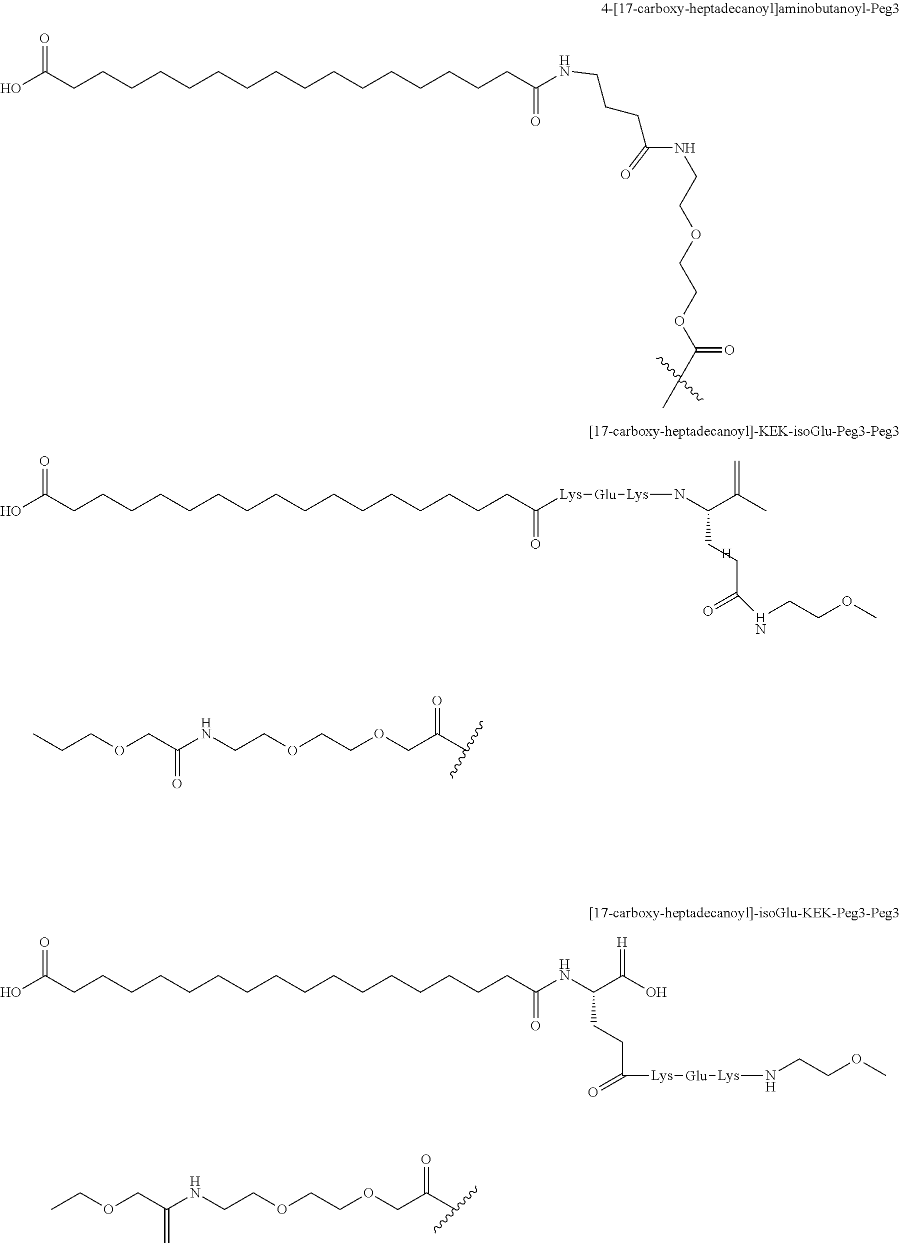

The skilled person will be well aware of suitable techniques for preparing the substituents employed in the context of the invention and conjugating them to the side chain of the appropriate amino acid in the dual agonist peptide. For examples of suitable chemistry, see WO98/08871, WO00/55184, WO00/55119, Madsen et al., J. Med. Chem. 50:6126-32 (2007), and Knudsen et al., J. Med Chem. 43:1664-1669 (2000), incorporated herein by reference.

In another aspect of the invention the dual agonist may be any of the agonists as described in WO2018/104560, which is incorporated herein by reference.

For example, the GLP-1/GLP-2 dual agonist may be a compound represented by the formula:

R¹—X*-U-R²

wherein:

R¹ is hydrogen (Hy), C₁₋₄ alkyl (e.g. methyl), acetyl, formyl, benzoyl or trifluoroacetyl;

R² is NH₂ or OH;

X* is a peptide of formula I:

(I)
H-X2-EG-X5-F-X7-X8-E-X10-X11-TIL-X15-X16-X17-A-
X19-X20-X21-FI-X24-WL-X27-X28-X29-KIT-X33

wherein

X2 is Aib or G;

X5 is S or T;

X7 is S or T;

X8 is S, E or D;

X10 is L, M or V;

X11 is A, N or S;

X15 is D or E

X16 is E, A or G;

X17 is Q, E, L or K;

X19 is A, V or S;

X20 is R or K;

X21 is D, L or E;

X24 is A, N or S;

X27 is I, Y, Q, H or K;

X28 is A, E, H, Y, L, K, Q, R or S;

X29 is H, Y, K or Q;

X33 is D or E;

U is absent or a sequence of 1-15 residues, each independently selected from K and k;

and wherein at least one of X5 and X7 is T;

or a pharmaceutically acceptable salt or solvate thereof.

### Synthesis of Dual Agonists

The dual agonists according to the invention may be synthesized according to the methods set out in International publication numbers WO2018/104560 and WO2018/104561, which are incorporated herein by reference.

Dual agonists may be synthesised by means of solid-phase or liquid-phase peptide synthesis methodology. In this context, reference may be made to WO 98/11125 and, among many others, Fields, G. B. et al., 2002, “Principles and practice of solid-phase peptide synthesis”. In: Synthetic Peptides (2nd Edition), and the Examples herein.

The dual agonist may be synthesized or produced in a number of ways, including for example, a method which comprises

(a) synthesizing the dual agonist by means of solid-phase or liquid-phase peptide synthesis methodology and recovering the synthesized dual agonist thus obtained; or

(b) expressing a precursor peptide sequence from a nucleic acid construct that encodes the precursor peptide, recovering the expression product, and modifying the precursor peptide to yield a compound of the invention.

The precursor peptide may be modified by introduction of one or more non-proteinogenic amino acids, e.g. Aib, Orn, Dap, or Dab, introduction of a lipophilic substituent Z¹ or Z¹—Z²— at a residue Ψ, introduction of the appropriate terminal groups R¹ and R², etc.

Expression is typically performed from a nucleic acid encoding the precursor peptide, which may be performed in a cell or a cell-free expression system comprising such a nucleic acid.

Analogues may be synthesised by means of solid-phase or liquid-phase peptide synthesis. In this context, reference is made to WO 98/11125 and, among many others, Fields, G B et al., 2002, “Principles and practice of solid-phase peptide synthesis”. In: Synthetic Peptides (2nd Edition).

For recombinant expression, the nucleic acid fragments encoding the precursor peptide will normally be inserted in suitable vectors to form cloning or expression vectors. The vectors can, depending on purpose and type of application, be in the form of plasmids, phages, cosmids, mini-chromosomes, or virus, but also naked DNA which is only expressed transiently in certain cells is an important vector. Preferred cloning and expression vectors (plasmid vectors) are capable of autonomous replication, thereby enabling high copy-numbers for the purposes of high-level expression or high-level replication for subsequent cloning.

In general outline, an expression vector comprises the following features in the 5′→3′ direction and in operable linkage: a promoter for driving expression of the nucleic acid fragment, optionally a nucleic acid sequence encoding a leader peptide enabling secretion (to the extracellular phase or, where applicable, into the periplasma), the nucleic acid fragment encoding the precursor peptide, and optionally a nucleic acid sequence encoding a terminator. They may comprise additional features such as selectable markers and origins of replication. When operating with expression vectors in producer strains or cell lines it may be preferred that the vector is capable of integrating into the host cell genome. The skilled person is very familiar with suitable vectors and is able to design one according to their specific requirements.

The vectors may be used to transform host cells to produce the precursor peptide. Such transformed cells can be cultured cells or cell lines used for propagation of the nucleic acid fragments and vectors, and/or used for recombinant production of the precursor peptides.

Preferred transformed cells are micro-organisms such as bacteria [such as the species Escherichia (e.g. E. coli), Bacillus (e.g. Bacillus subtilis), Salmonella, or Mycobacterium (preferably non-pathogenic, e.g. M. bovis BCG), yeasts (e.g., Saccharomyces cerevisiae and Pichia pastoris), and protozoans. Alternatively, the transformed cells may be derived from a multicellular organism, i.e. it may be fungal cell, an insect cell, an algal cell, a plant cell, or an animal cell such as a mammalian cell. For the purposes of cloning and/or optimised expression it is preferred that the transformed cell is capable of replicating the nucleic acid fragment of the invention. Cells expressing the nucleic fragment can be used for small-scale or large-scale preparation of the peptides of the invention.

When producing the precursor peptide by means of transformed cells, it is convenient, although far from essential, that the expression product is secreted into the culture medium.

### Pharmaceutical Compositions and Administration

The agonist combination according to the present invention may be in the form of a composition comprising a GLP-1 agonist and/or GLP-2 agonist, or dual agonist, or a pharmaceutically acceptable salt or solvate thereof, together with a carrier. In one embodiment of the invention, the composition is a pharmaceutical composition and the carrier is a pharmaceutically acceptable carrier. The pharmaceutical composition comprising an agonist, or dual agonist according to the invention, or a salt or solvate thereof, may be together with a carrier, excipient or vehicle. Accordingly, the agonist or dual agonist, or salts or solvates thereof, especially pharmaceutically acceptable salts or solvates thereof, may be formulated as compositions or pharmaceutical compositions prepared for storage or administration, and which comprise a therapeutically effective amount of an agonist, dual agonist, or a salt or solvate thereof.

Suitable salts formed with bases include metal salts, such as alkali metal or alkaline earth metal salts, for example sodium, potassium or magnesium salts; ammonia salts and organic amine salts, such as those formed with morpholine, thiomorpholine, piperidine, pyrrolidine, a lower mono-, di- or tri-alkylamine (e.g., ethyl-tert-butyl-, diethyl-, diisopropyl-, triethyl-, tributyl- or dimethylpropylamine), or a lower mono-, di- or tri-(hydroxyalkyl)amine (e.g., mono-, di- or triethanolamine). Internal salts may also be formed. Similarly, when a compound of the present invention contains a basic moiety, salts can be formed using organic or inorganic acids. For example, salts can be formed from the following acids: formic, acetic, propionic, butyric, valeric, caproic, oxalic, lactic, citric, tartaric, succinic, fumaric, maleic, malonic, mandelic, malic, phthalic, hydrochloric, hydrobromic, phosphoric, nitric, sulphuric, benzoic, carbonic, uric, methanesulphonic, naphthalenesulphonic, benzenesulphonic, toluenesulphonic, p-toluenesulphonic (i.e. 4-methylbenzene-sulphonic), camphorsulphonic, 2-aminoethanesulphonic, aminomethylphosphonic and trifluoromethanesulphonic acid (the latter also being denoted triflic acid), as well as other known pharmaceutically acceptable acids. Amino acid addition salts can also be formed with amino acids, such as lysine, glycine, or phenylalanine.

In one embodiment, a pharmaceutical composition is one wherein the agonist or dual agonist is in the form of a pharmaceutically acceptable acid addition salt.

As will be apparent to one skilled in the medical art, a “therapeutically effective amount” of an agonist or dual agonist or pharmaceutical composition thereof according to the present invention will vary depending upon, inter alia, the age, weight and/or gender of the subject (patient) to be treated. Other factors that may be of relevance include the physical characteristics of the specific patient under consideration, the patient's diet, the nature of any concurrent medication, the particular compound(s) employed, the particular mode of administration, the desired pharmacological effect(s) and the particular therapeutic indication. Because these factors and their relationship in determining this amount are well known in the medical arts, the determination of therapeutically effective dosage levels, the amount necessary to achieve the desired result of treating and/or preventing and/or remedying malabsorption and/or low-grade inflammation described herein, as well as other medical indications disclosed herein, will be within the ambit of the skilled person.

As used herein, the term “a therapeutically effective amount” refers to an amount which reduces symptoms of a given condition or pathology, and preferably which normalizes physiological responses in an individual with that condition or pathology. Reduction of symptoms or normalization of physiological responses can be determined using methods routine in the art and may vary with a given condition or pathology. In one aspect, a therapeutically effective amount of one or more agonist or dual agonist, or pharmaceutical compositions thereof, is an amount which restores a measurable physiological parameter to substantially the same value (preferably to within 30%, more preferably to within 20%, and still more preferably to within 10% of the value) of the parameter in an individual without the condition or pathology in question.

An agonist combination for use according to the invention may be administered to the patient about 7, 6, 5, 4, 3, 2, or 1 day after surgery, for example about 48 hours after surgery, about 44 hours after surgery, about 40 hours after surgery, about 36 hours after surgery, about 32 hours after surgery, about 32 hours after surgery, about 28 hours after surgery, about 24 hours after surgery, about 20 hours after surgery, about 16 hours after surgery, about 12 hours after surgery, about 8 hours after surgery, about 4 hours after surgery, about three hours after surgery, about two hours after surgery, or about one hour after surgery.

In one aspect the agonist combination may be administered about 24 hours after surgery.

After surgery, the agonist combination may be administered as a single dose administration. Alternatively, the agonist combination may be administered as a multi dose administration.

In one aspect the agonist combination may also be administered to said patient before surgery. For example, the dual agonist combination may be administered about 7, 6, 5, 4, 3, 2, or 1 day before surgery, for example 48 hours before surgery, about 44 hours before surgery, about 40 hours before surgery, about 36 hours before surgery, about 32 hours before surgery, about 32 hours before surgery, about 28 hours before surgery, about 24 hours before surgery, about 20 hours before surgery, about 16 hours before surgery, about 12 hours before surgery, about 8 hours before surgery, about 4 hours before surgery, about three hours before surgery, about two hours before surgery, or about one hour before surgery.

### Dosing

In one embodiment of the invention, administration of an agonist combination according to the present invention is commenced at lower dosage levels, with dosage levels being increased until the desired effect of preventing/treating the relevant medical indication is achieved. This would define a therapeutically effective amount. For the agonist combination according to the present invention, alone or as part of a pharmaceutical composition, such human doses of the active agonist or dual agonist may be between about 0.1 pmol/kg and 500 μmol/kg body weight, between about 0.01 pmol/kg and 300 μmol/kg body weight, between 0.01 pmol/kg and 100 μmol/kg body weight, between 0.1 pmol/kg and 50 μmol/kg body weight, between 1 pmol/kg and 10 μmol/kg body weight, between 5 pmol/kg and 5 μmol/kg body weight, between 10 pmol/kg and 1 μmol/kg body weight, between 50 pmol/kg and 0.1 μmol/kg body weight, between 100 pmol/kg and 0.01 μmol/kg body weight, between 0.001 μmol/kg and 0.5 μmol/kg body weight, between 0.05 μmol/kg and 0.1 μmol/kg body weight.

In one aspect the dose is in the range of about 50 pmol/kg to 500 nmol/kg, for example about 60 pmol/kg to 400 nmol/kg, about 70 pmol/kg to 300 nmol/kg, about 80 pmol/kg to 200 nmol/kg, about 90 pmol/kg to 100 nmol/kg.

In one aspect the dose is in the nmol range, for example between 1 nmol/kg and 100 nmol/kg, between 1 nmol/kg and 90 nmol/kg, between 1 nmol/kg and 80 nmol/kg, between 1 nmol/kg and 70 nmol/kg, between 1 nmol/kg and 60 nmol/kg, between 1 nmol/kg and 50 nmol/kg, between 1 nmol/kg and 40 nmol/kg, between 1 nmol/kg and 30 nmol/kg, between 1 nmol/kg and 20 nmol/kg, or between 1 nmol/kg and 10 nmol/kg. The therapeutic dosing and regimen most appropriate for patient treatment will of course vary with the disease or condition to be treated, and according to the patient's weight and other parameters. Without wishing to be bound by any particular theory, it is expected that doses, in the pmol/kg or nmol/kg range, and shorter or longer duration or frequency of treatment may produce therapeutically useful results, such as a statistically significant increase particularly in small bowel mass. In some instances, the therapeutic regimen may include the administration of maintenance doses appropriate for preventing tissue regression that occurs following cessation of initial treatment. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

In one aspect the agonist combination according to the invention is administered intravenously, in a continuous infusion.

In one aspect the patient is an adult. In one aspect the patient is a neonate. In one aspect the patient is a child.

### Bowel Resection

The present invention relates to the field of bowel resection.

A bowel resection is a surgery to remove any part of the bowel. This includes the small intestine, large intestine, or rectum. A bowel resection according to the invention may be performed as required for a particular patient.

The large intestine, also known as the large bowel, is the last part of the gastrointestinal tract and of the digestive system in vertebrates. Water is absorbed here and the remaining waste material is stored as faeces before being removed by defecation.

In humans, the large intestine begins in the right iliac region of the pelvis, just at or below the waist, where it is joined to the end of the small intestine at the cecum, via the ileocecal valve. It then continues as the colon ascending the abdomen, across the width of the abdominal cavity as the transverse colon, and then descending to the rectum and its endpoint at the anal canal. Overall, in humans, the large intestine is about 1.5 metres (5 ft) long, which is about one-fifth of the whole length of the gastrointestinal tract.

The small intestine or small bowel is an organ in the gastrointestinal tract where most of the end absorption of nutrients and minerals from food takes place. It lies between the stomach and large intestine, and receives bile and pancreatic juice through the pancreatic duct to aid in digestion.

The small intestine has three distinct regions—the duodenum, jejunum, and ileum. The duodenum, the shortest, is where preparation for absorption through small finger-like protrusions called villi begins. The jejunum is specialized for the absorption through its lining by enterocytes: small nutrient particles which have been previously digested by enzymes in the duodenum. The main function of the ileum is to absorb vitamin B12, bile salts, and whatever products of digestion were not absorbed by the jejunum.

Different types of bowel resections may be performed to remove different parts of the bowel. Each type of bowel resection is named based on what it removes.

A segmental small bowel resection removes part of the small intestine. Some of the mesentery (a fold of tissue that supports the small intestine) and lymph nodes in the area may also be removed.

A segmental small bowel resection is used to remove tumours in the lower part of the duodenum, in the jejunum or in the ileum if the cancer is only in these structures or if it has spread just beyond the small intestine.

A right hemicolectomy removes:
part of the ileum
the cecum
the ascending colon (the first part of the colon)
the hepatic flexure (the bend in the colon near the liver)
the first part of the transverse colon (the middle of the colon)
the appendix

A right hemicolectomy is used to remove tumours in the right colon, including the cecum and ascending colon. It may also be done to remove tumours of the appendix.

An extended right hemicolectomy removes all of the transverse colon. It may be done to remove tumours in the hepatic flexure or transverse colon.

A transverse colectomy removes the transverse colon. This surgery may be done to remove a tumour in the middle of the transverse colon when the cancer hasn't spread to any other parts of the colon. Sometimes an extended right hemicolectomy is done instead.

A left hemicolectomy removes:
part of the transverse colon
the splenic flexure (the bend in the colon near the spleen)
the descending colon
part of the sigmoid colon

A low anterior resection removes the sigmoid colon and part of the rectum. It is used to remove tumours in the middle or upper part of the rectum.

A proctocolectomy (also called a proctectomy) removes all of the rectum and part of the sigmoid colon. Coloanal anastomosis joins the remaining colon to the anus.

This surgery is used to remove tumours in the lower part of the rectum. Often a low anterior resection or an abdominoperineal resection is done instead.

An abdominoperineal resection removes the rectum, anus, anal sphincter and muscles around the anus. It is used to remove tumours that are close to the anus or have grown into the muscles around the anus.

A subtotal or total colectomy removes part or all of the colon. If most of the colon is removed, it is called a subtotal or partial colectomy. If all of the colon is removed, including the cecum and the appendix, it is called a total colectomy.

A subtotal or total colectomy is done:
when there is cancer on both the right and left sides of the colon
as a way to prevent colorectal cancer (called a prophylactic colectomy) for some people with familial adenomatous polyposis (FAP) or Lynch syndrome (also called hereditary non-polyposis colon cancer or HNPCC)
to remove a colon that is damaged by an inflammatory bowel disease

Depending on the type of colectomy done, the surgeon may also need to do a colostomy or an ileostomy.

In one aspect of the invention, about 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, or 20.0% or more, such as about 30, 40, 50, 60, 70, 80 or 90% of the bowel of said patient is removed.

The length of bowel removed may depend on the total length of the bowel of the patient, for example about 5 cm or more may be removed. In one aspect at least about 30 cm of bowel is retained.

### Short Bowel Syndrome (SBS)

In one aspect of the invention the patient has short bowel syndrome (SBS).

SBS usually results from surgical resection of some or most of the small intestine for conditions such as Crohn's disease, mesenteric infarction, volvulus, trauma, congenital anomalies, and multiple strictures due to adhesions or radiation.

SBS patients suffer from malabsorption that may lead to malnutrition, dehydration and weight loss. Some patients can maintain their protein and energy balance through hyperphagia; more rarely they can sustain fluid and electrolyte requirements to become independent from parenteral fluid.

Short bowel syndrome (SBS) is anatomically defined as that symptom complex which occurs in adults who have less than 200 centimeters of combined jejunum-ileum following small bowel resection. As the intestinal length in children is linked to the state of growth, a definition of a short bowel in absolute terms has not been devised. The need for intravenous supplementation or a residual short bowel length of less than 25% expected for gestational age are suggested definitions of a short bowel in children

The syndrome is characterized by diarrhea, weight loss, dehydration, malnutrition, and malabsorption of macro- and micronutrients. Note that some patients who have had extensive bowel resections leaving them with more than 200 centimeters of small intestine can develop symptoms indistinguishable from those who fulfill the technical criteria for SBS. This situation occurs when the remaining bowel is diseased as, for example, in patients with Crohn's disease or radiation enteritis. These latter patients are generally managed like the standard SBS patient.

### Advantageous Effects

As described herein, the present invention is advantageous in that it may lead to improved adaptation of the bowel following resection, and improved recovery of the patient. The present invention may improve, or shorten the time to, intestinal rehabilitation.

As demonstrated in the present Examples, the composition according to the present invention may lead to increased growth of intestinal villi, increased intestinal length, increased cell growth (trophic effect), and weight gain following surgery.

As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein.

In one aspect the composition for use according to the present invention may lead to increased length of the intestine. The increase in length of the intestine in patients receiving the composition may be about 5, 10, 15, 20, 25, 30, 35, 40, 45, 50, 55, 60, 65, 70, 75, 80, 85, 90, 95 or 100% compared to patients to whom the composition is not administered. The increase in length may be in the ileum.

In one aspect the composition for use according to the present invention may lead to an increase in body weight following the surgical resection of the bowel. For example, relative body weight may be increased at six or more days post-surgery compared to weight at 1, 2, 3, 4, or 5 days post-surgery.

In one aspect the efficiency of nutrient uptake may be achieved using the composition for use according to the present invention, for example the patient may gain more weight from a specified amount of food or other nutrition compared with patients to whom the composition is not administered.

In one aspect the composition for use according to the invention is effective to increase villus growth, increase intestinal length, increase cell growth (trophic effect), increase weight gain, increase efficiency of nutrient uptake, increase mucosal height, and/or increase intestinal weight.

In one aspect of the invention intestinal weight is increased. In one aspect the increase may be about 5, 10, 15, 20, 25, 30, 35, 40, 45, 50, 55, 60, 65, 70, 75, 80, 85, 90, 95 or 100%.

In one aspect the composition for use according to the invention may results in cell growth, or hypertrophy, or cells in the bowel.

In one aspect the diameter of the bowel may be increased. In one aspect the increase may be about 5, 10, 15, 20, 25, 30, 35, 40, 45, 50, 55, 60, 65, 70, 75, 80, 85, 90, 95 or 100%.

In one aspect the villus to crypt ratio is increased. In one aspect growth of the villi is increased. In one aspect the increase may be about 5, 10, 15, 20, 25, 30, 35, 40, 45, 50, 55, 60, 65, 70, 75, 80, 85, 90, 95 or 100%.

In one aspect the mucosal height is increased. In one aspect the increase may be about 5, 10, 15, 20, 25, 30, 35, 40, 45, 50, 55, 60, 65, 70, 75, 80, 85, 90, 95 or 100%.

The increase in bowel length may be about 5, 10, 15, 20, 25, 30, 35, 40, 45, 50, 55, 60, 65, 70, 75, 80, 85, 90, 95 or 100%.

In one aspect of the invention the patient may have improved survival.

One skilled in the art will be aware of how to measure such parameters using techniques known in the art. Standard techniques and methods may be used to measure such parameters, such as those described in the present Examples, e.g. histological techniques and visual measurement and assessment, which will be known to one skilled in the art.

### Methods of Treatment

The present invention encompasses methods of treatment and therapeutic uses corresponding to all of aspects of the composition for use as described herein.

As such, the invention provides a method for treating a patient who has undergone surgical resection of the bowel, wherein said method comprises administering an agonist combination as described herein to said patient within 7 days of said surgical resection.

The invention also encompasses the use of an agonist combination as described herein in the manufacture of a medicament for use in the treatment of a patient who has undergone surgical resection of the bowel, wherein said GLP-1/GLP-2 dual agonist is administered to said patient within 7 days of said surgical resection.

The invention also provides use of an agonist combination as described herein in the treatment of a patient who has undergone surgical resection of the bowel, wherein GLP-1/GLP-2 dual agonist is administered to said patient within 7 days of said surgical resection.

Methods of treatment corresponding to all aspects of the agonist combination for use as described herein are encompassed by the present invention. Similarly, uses corresponding to all aspects of the agonist combination for use as described herein are encompassed by the present invention.

### Clinical Nutrition

Clinical nutrition deals with the prevention, diagnosis, and management of nutritional and metabolic changes related to acute and chronic diseases and conditions caused by a lack or excess of energy and nutrients. Nutrition therapy describes how nutrients are provided to treat any nutritional-related condition. Nutrition or nutrients can be provided orally (regular diet, therapeutic diet, oral nutritional supplements), via enteral tube feeding, or alternatively as parenteral nutrition. Medical nutrition therapy relates to oral nutritional supplements, enteral tube feeding, i.e. enteral nutrition, and parenteral nutrition.

In one aspect of the invention the patient receives oral nutrition (regular diet, therapeutic diet, oral nutritional supplements) following surgical bowel resection.

In one aspect of the invention the patient receives enteral nutrition following surgical bowel resection.

Enteral tube feeding or nutrition is nutrition therapy given via a tube or stoma into the intestinal tract distal to the oral cavity. The tube may be inserted via the nose (naso-gastric, naso-jejunal or naso-post pyloric tube feeding). Enteral nutrition may alternatively be delivered via a stoma that is inserted endoscopically into the stomach (percutaneous endoscopic gastrostomy (PEG)) or with a jejunal extension (PEG-J) or into the jejunum (percutaneous endoscopic jejunostomy (PEJ)). Alternatively, the tube may also be placed surgically (surgical gastrostomy or jejunostomy).

Total enteral tube feeding, or total enteral nutrition, denotes the situation where all nutrient needs are provided through a feeding tube without significant oral or parenteral intake.

Supplemental enteral tube feeding denotes nutrition given to patients whose oral intake of food and fluids is inadequate for reaching their defined nutritional target alone.

When enteral feeding is used outside the hospital it is called home enteral nutrition (HEN) or home enteral tube feeding (HETF). HEN/HETF may be provided either as total or supplemental enteral nutrition

Suitable enteral nutrition compositions or formulas will be known to one of skill in the art. Nutrition products that are delivered via enteral feeding are defined in EU legislation as “foods for special medical purposes” (FSMPs). Such products are specially processed or formulated and intended for the dietary management of patients under medical supervision.

By way of example, standard formulas (whole protein formulas) are designed for adults or children who have normal digestion. Standard formulas include all of the nutrients required to maintain health. Some standard formulas can be used for both enteral feeding and as an oral supplement. They can contain added ingredients, such as fibre, for digestive health and bowel management.

Peptide formulas (semi-elemental formulas) are nutritionally complete, which means they contain all the essential nutrients needed. However, unlike standard formulas, some of the components, such as protein are “broken down” into smaller components to make them easier to digest. Peptide formulas are easier for the digestive system to digest and absorb, making them better suited for adults and children with digestive problems, including malabsorption, short bowel syndrome, inflammatory bowel disease, cystic fibrosis and other conditions that can cause problems with absorbing nutrients.

In one aspect of the invention the enteral composition is a peptide formula composition.

Specialised enteral formulas are also available for adults and children with special nutritional needs, such as diabetes, kidney failure, respiratory disease, or liver disorders. The enteral formula should be selected by a doctor or a dietitian who is familiar with the various formulas.

Various enteral nutritional compositions are available with different contents of nutrition, depending on the patient's needs and clinical situation. Examples of commercially available enteral nutritional compositions include: Isocal (Novartis), Nutren 1.0 (Nestle), Osmolite 1.0 (Ross), Fibersource 1.2 (Novartis), Jevity 1.2 (Ross), Osmolite 1.2 (Ross), probalance (Nestle), Isosource 1.5 (Novartis), Jevity 1.5 (Ross), Nutren 1.5 (Nestle), Deliver 2.0 (Novartis), Novasource 2.0 (Novartis), Nutren 2.0 (Nestle) and TwoCal HN (Ross).

For example, the protein content of the composition may be from 10 to 80%. The protein component may be made with casein, soy, hydrolyzed protein with added amino acids, or free amino acids alone.

The carbohydrate content of the composition may be from 10 to 90%. The carbohydrate component may be made with starch, glucose polymers, and/or disaccharides such as sucrose.

The fat content may be from 10% to 50%. The fat content may be made with long-chain triglycerides, medium chain triglycerides and fish or other specialty oils

A standard enteral nutritional composition may contain the following:
Protein content: 10-15%
Carbohydrate content: 50-60%
Fat content: 30-35%

In one aspect the enteral formula is free of lactose and/or gluten.

In one aspect of the invention is provided the composition for use according to the invention, wherein said patient receives enteral nutrition within about 48 hours of said surgical resection, for example within about 44, 40, 36, 32, 28, 24, 20, 16, 12, 18, 4, 3, 2, or 1 hour of said surgical resection In a preferred aspect said patient receives enteral nutrition within about 24 hours of said surgical resection.

In one aspect the enteral nutrition may constitute less than about 80% of the recommended daily intake. For example, the enteral nutrition may constitute less than about 70, 65, 60, 55, 50, 45, 40, 35, 30, 25, 20, 15 or 10% of the recommended daily intake.

Nutrient concentrations of standard enteral nutrition compositions may vary from 1.0-2.0 kcal/mL. In general, energy, protein and micronutrient needs are covered by 1.5 L of standard enteral formula.

In one aspect said enteral nutrition may provide to the patient at least about 200, 300, 400, 500, 600, 700, 800, 900 or more kcal per day. In one aspect said enteral nutrition may provide to the patient at least about 1000 kcal per day. For example, said enteral nutrition may provide at least about 1100 1200, 1300, 1400, 1500, 1600, 1700, 1800, 1900, 2000, 2100, 2200, 2300, 2400 or 2500 kcal per day.

In one aspect said patient does not receive enteral nutrition prior to said surgical resection.

In one aspect the patient may receive parenteral nutrition, for example total parenteral nutrition, before and/or after surgery.

Suitable, parenteral nutrition compositions or formulations will be known to one skilled in the art.

In a further aspect there is provided a kit comprising a composition comprising an agonist combination according to the invention for use in the treatment of a patient who has undergone surgical resection of the bowel, wherein said agonist combination is administered to said patient within 48 hours of said surgical resection.

The following examples are provided to illustrate preferred aspects of the invention and are not intended to limit the scope of the invention in any way.

### EXAMPLES

### Preparation of Short Bowel Syndome (SBS) Mice

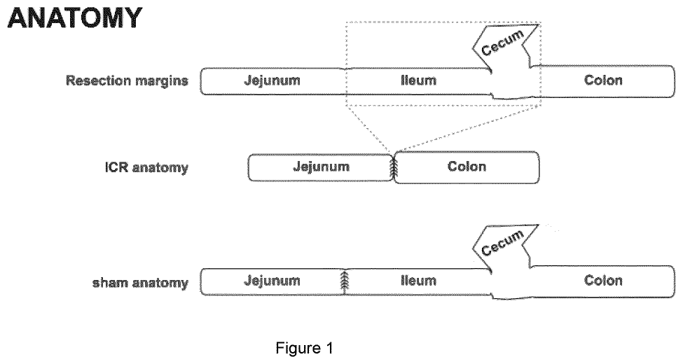

Adult, male C57BL/6J mice with ˜30 g body weight of ˜4 months, received a 12-cm ileum and cecum resection (ICR) with jejunocolonic anastomosis (see FIG. 1, middle). The mice were then randomly assigned to either Cpd 18 or vehicle treatment As control for the abdominal surgery, the sham group was included which had laparotomy, bowel exposure, transection and end-to-end anastomosis but no resection (see FIG. 1, bottom). To prevent intestinal obstruction, all mice were switched to liquid diet 2 days before surgery and maintained on this diet until the end of the experiment. Throughout the experiment, animals were singly housed. All mice were studied with the intention to survive to day 14.

Briefly, mice were anesthetized by i.p. injection of ketamine (100 mg/kg bw) and xylazine (15 mg/kg bw) and orally intubated and ventilated. After midline incision, the distal small bowel and the cecum were exposed and then transected 12 cm proximal to the ileocecal junction and immediately distal to the cecum. The resected bowel was removed, and the length of the specimen was measured. An end-to-end, jejuno-colonic anastomosis was created and the abdomen was closed. In the sham group, laparotomy, bowel exposure, transection and end-to-end anastomosis but no resection was performed. Immediately after surgery, all mice were weighed and received 1 ml isotonic saline subcutaneously and carprofen 5 mg/kg body weight. All surgical procedures were performed by the same surgeon. Mice were kept in a heated terrarium (29° C.) for 4 h and then returned to individual cages with free access to liquid food and water. The liquid diet was provided in feeding tubes. Mice were allowed to recover for a day after surgery, and then injected once daily with 30 nmol/kg body weight of Cpd 18. 50 mM Histidine buffer pH 6, 200 mM Mannitol served as vehicle control.

Mice were scored for wellbeing daily as described (Berlin et al. 2019). If the wellness score dropped to below 4 pts, or when weight loss exceeded 20%, the mouse was immediately killed and not analyzed further. On day 14 animals were animals were sacrificed by cervical dislocation and the small bowel excised for further study.

### Example 1: Observations on the Mice Used in the Following Examples

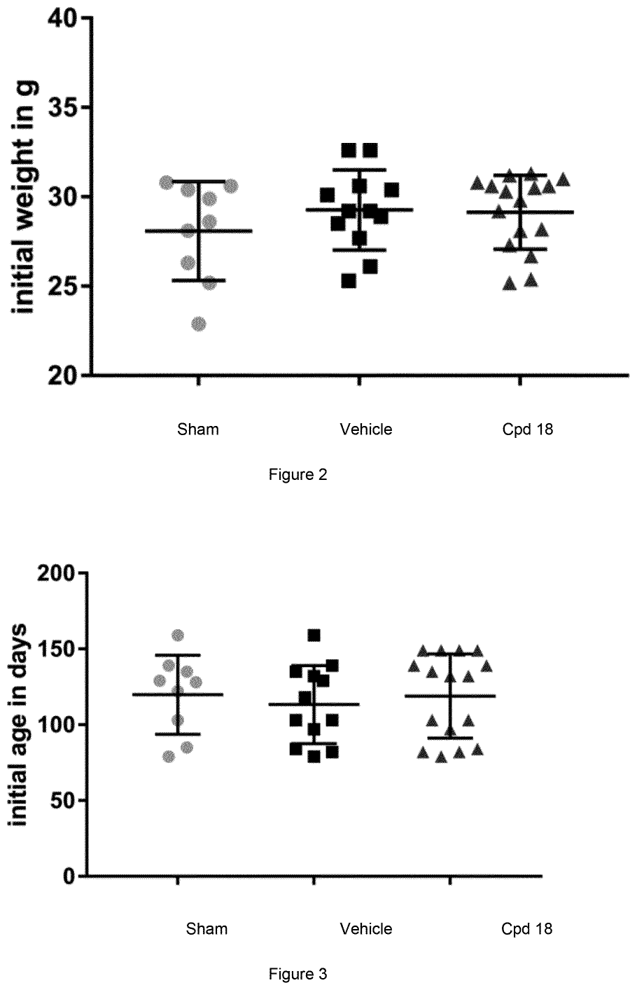

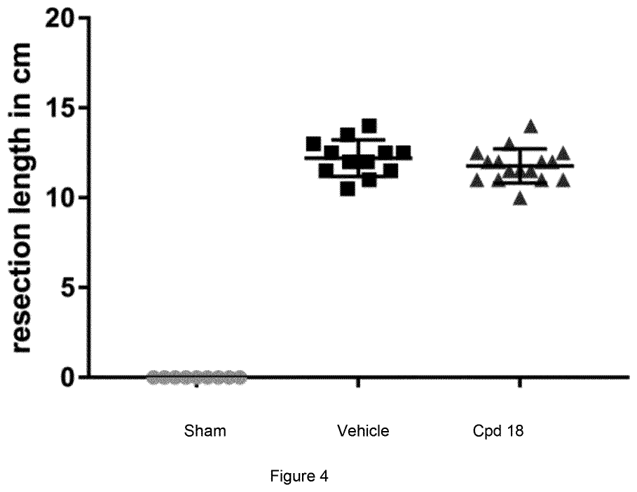

Sham and SBS mice were prepared as described under Preparation of Short Bowel Syndome (SBS) mice. The initial weight and initial age of the mice used are shown in FIGS. 2 and 3. As seen from the figures there were no differences between the sham, vehicle or Cpd 18 (Compound 18) groups for the initial weight of the mice prior to surgery (FIG. 2), and no differences between the sham, vehicle or Cpd 18 groups for the initial age of the mice prior to surgery (FIG. 3). There was an equal resection length between the vehicle or Cpd 18 groups, whilst there was no resection performed for the sham group, only an anastomosis (FIG. 4).

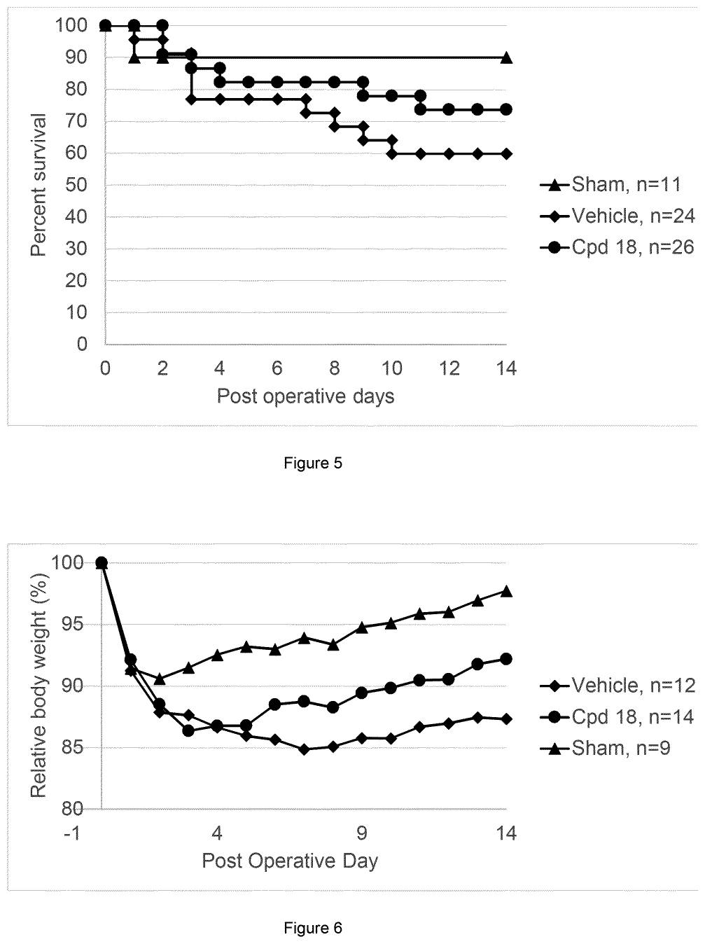

FIG. 5 shows that vehicle treated mice had the lowest survival rate after 14 days (60%), whilst treatment with Cpd 18 gave an increased rate of survival (74%) as compared to vehicle treatment, sham operated animals had the highest survival rate (90%).

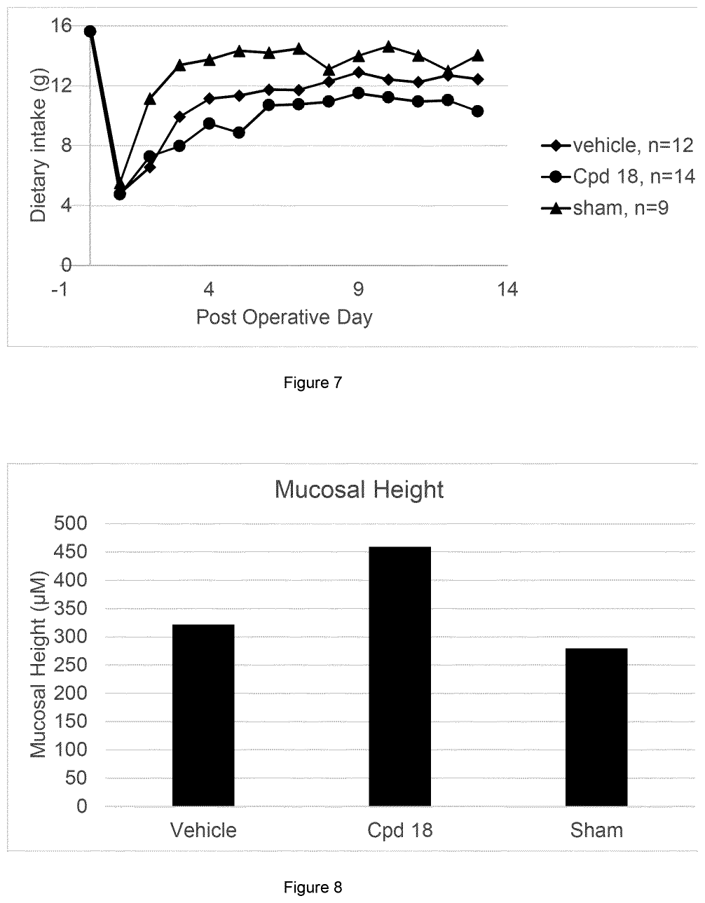

Body weight (FIG. 6) and food consumption (FIG. 7) was measured daily. The feeding tubes were weighed when replenished after 24 h in the cage.

As seen from FIG. 6, all groups dropped to approximately 90% of the relative starting weight one day after surgery. The relative body weight of the sham group started to increase towards starting weight after just 2 days, whilst the group treated with Cpd 18 differed from the vehicle group after six days onwards by starting to increase towards the initial weight, the vehicle group stayed relatively flat even after 14 days (FIG. 6). The increase in relative body weight in Cpd 18 treated group as compared to the vehicle group was despite dietary intake (FIG. 7) being lower in the Cpd 18 treated animals from day 2 onwards as compared to vehicle.

### Example 2: Effect of Cpd 18 in SBS Mouse Model on Mucosal Height

Sham and SBS mice were prepared as described under Preparation of Short Bowel Syndome (SBS) mice. For histological quantification, 1-cm intestinal segments were immediately placed in MorFFFix (Morphisto®, Frankfurt am Main, Germany) for 24 h and then paraffin-embedded as described under the paragraphs histology and immunofluorescence (Reiner et al. 2020). In total, 5-μm transverse sections were HE-stained and then used for morphometric analysis. In a blinded manner, the mucosal height was determined by measuring it per sample at 5 well-oriented full-length crypt-villus-units from the crypt base to the villus tip using an Axio Observer inverted microscope (Zeiss) and ZEN 2.3 software. Bowel diameter was measured from the same samples from serosa to serosa in two dimensions.

FIG. 8 shows the mucosal height for sham (n=4), Vehicle (n=4) and Cpd 18 (n=6). Vehicle treated animals had a slight increase in measured mucosal height as compared to sham, whilst Cpd 18 treated animals had approximately double the mucosal height of sham treated animals.

### Example 3: Effect of Compound 18 in SBS Mouse Model on Bowel Diameter

Sham and SBS mice were prepared as described under Preparation of Short Bowel Syndome (SBS) mice and stained as described in example 2. Bowel diameter was measured from the same samples from serosa to serosa in two dimensions.

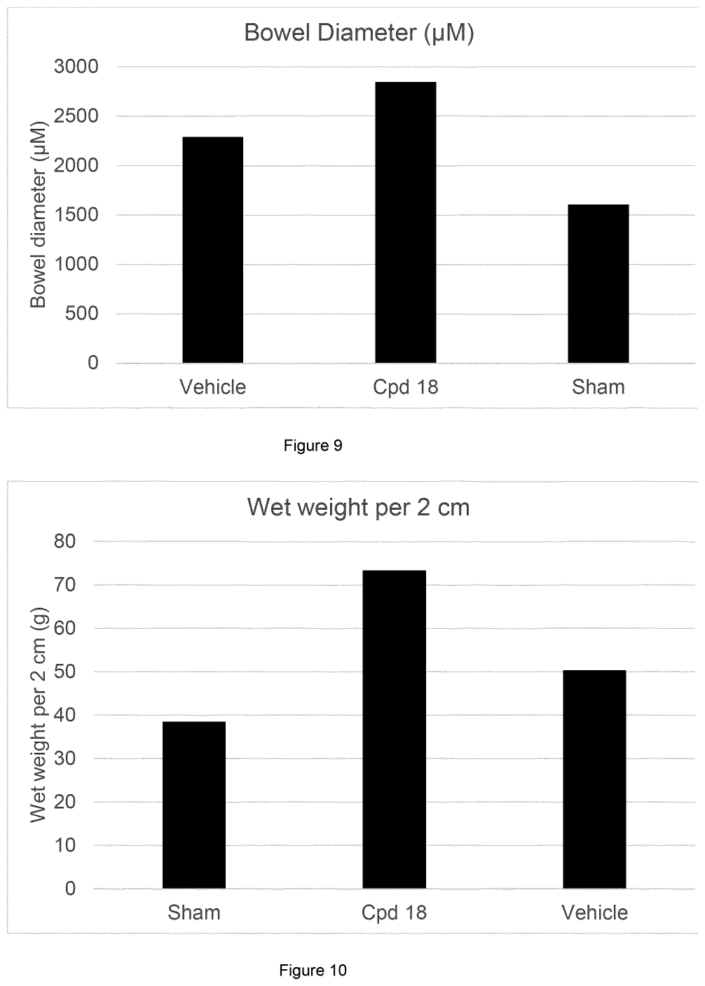

FIG. 9 shows the bowel diameter for sham (n=4), Vehicle (n=4) and Cpd 18 (n=6). Vehicle treated animals had an increase in measured bowel diameter as compared to sham, whilst Cpd 18 treated animals had approximately double the bowel diameter of sham treated animals.

### Example 4: Effect of Compound 18 in SBS Mouse Model on Jejunum Weight

Sham and SBS mice were prepared as described under Preparation of Short Bowel Syndome (SBS) mice. At termination on day 14, 2 cm of proximal Jejunum was obtained from each animal. The sample was taken and placed in a pre-weighed empty 2.0 ml tube. The tube with fresh Jejunum was immediately closed, weighed, dried overnight at 80° C., and then weighed with exsiccated Jejunum. Water content and dry weight was then derived and calculated.

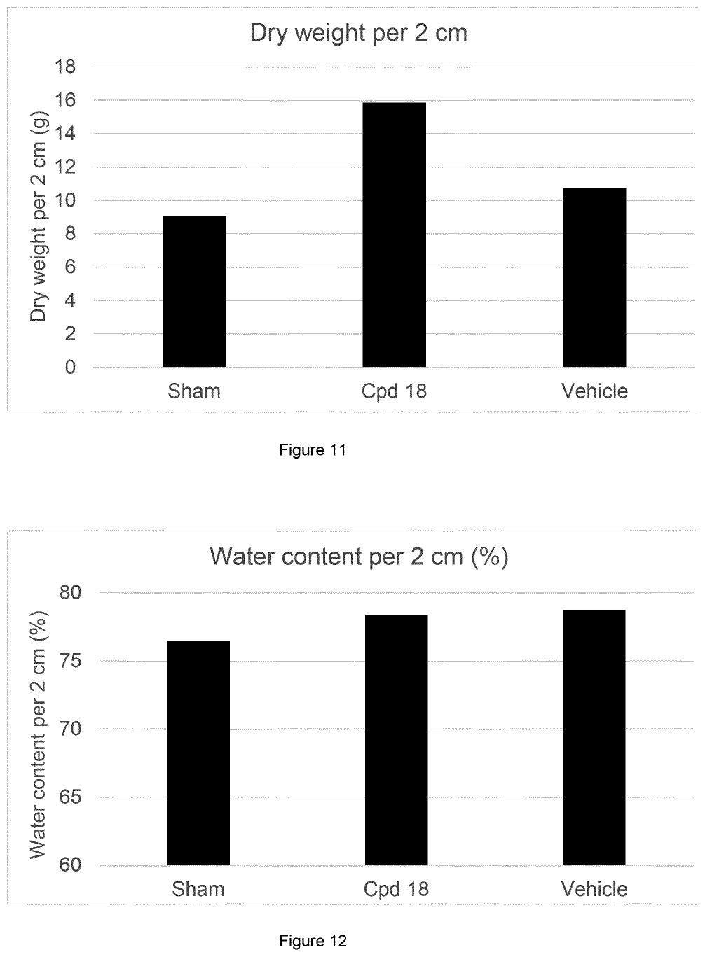

FIG. 10-12 show the intestine weight for sham (n=6), Vehicle (n=8) and Cpd 18 (n=10). The wet weight of 2 cm sections of resected intestine on day 14 showed that as compared to sham (38 g) vehicle treated animals increased (49 g), whilst Cpd 18 treated had approximately double the wet weight (73 g) of the sham group (FIG. 10). When these intestinal sections were desiccated the same increases in mass were measured (FIG. 11). When the percentage of water content per 2 cm was calculated, then each group had an equal water content of the intestine (FIG. 12) indicating that Cpd18 had an effect on intestinal growth.

### Example 5: Histology of Intestine

Sham and SBS mice were prepared as described under Preparation of Short Bowel Syndome (SBS) mice and stained as described in example 2.

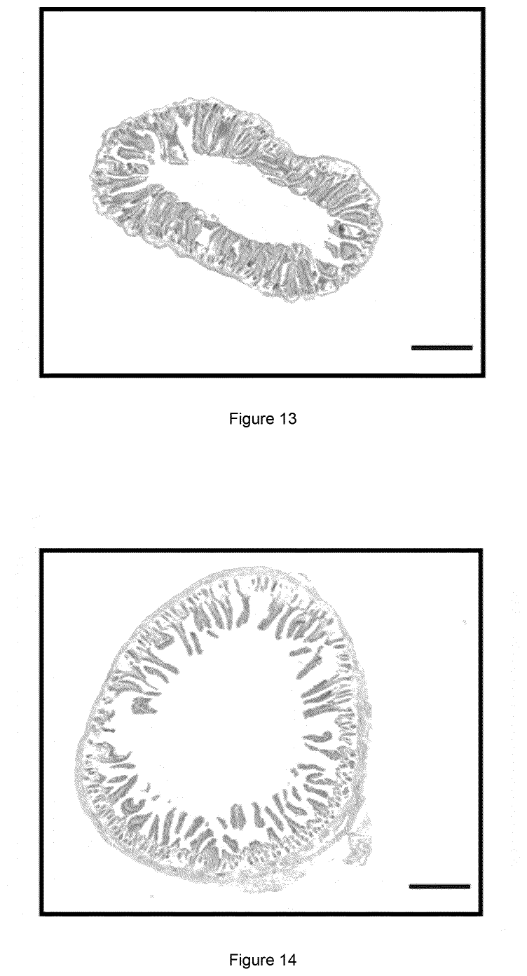

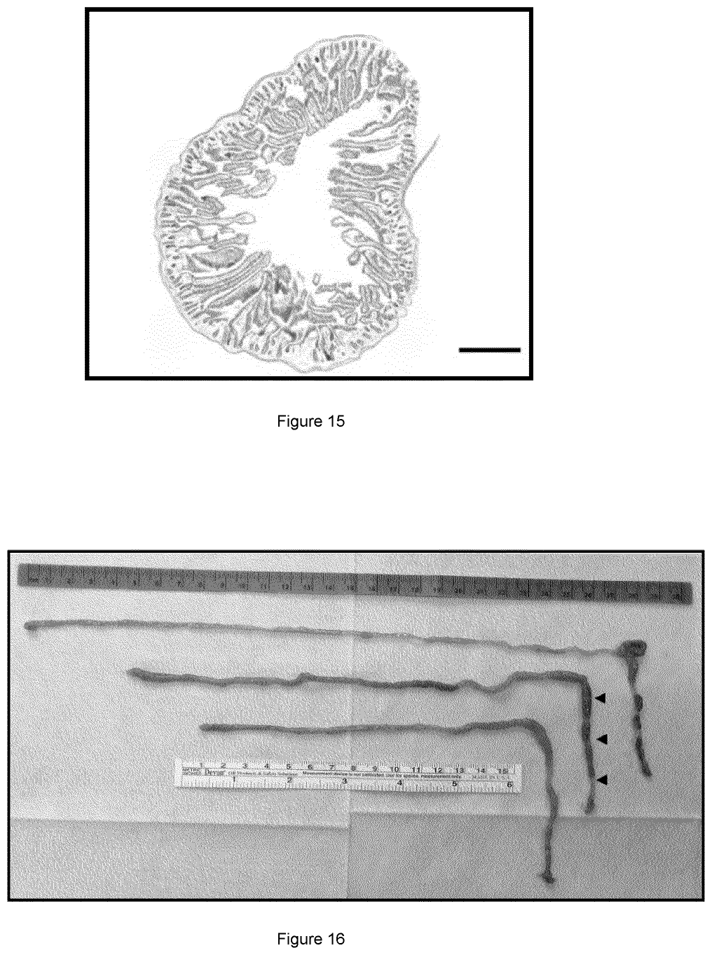

FIGS. 13-15 show representative examples of HE stained transverse sections through the bowel, FIG. 13 is from sham group, FIG. 14 is from vehicle group and FIG. 15 is from Cpd 18 treated group.

### Example 6: Effect of Compound 18 in SBS Mouse Model on Length of Intestine

Sham and SBS mice were prepared as described under Preparation of Short Bowel Syndome (SBS) mice.

FIGS. 16 show representative examples of the bowel removed on day 14 of the trial following sacrifice of the mice. The upper intestine is from a mouse from the sham group, the middle intestine is from a mouse from the Cpd 18 treated group and the lower intestine from the vehicle group. An increase in bowel length and thickness is seen in the example from the Cpd 18 treated mouse compared to the intestine from sham group mouse. Also visible in this figure are solid feces in the sham and Cpd 18 treated intestine, indicated by the black arrows, which are absent in the vehicle treated intestine.

### REFERENCES

Berlin P, et al. Villus Growth, Increased Intestinal Epithelial Sodium Selectivity, and Hyperaldosteronism Are Mechanisms of Adaptation in a Murine Model of Short Bowel Syndrome. Dig Dis Sci. 2019 May; 64(5):1158-1170. doi: 10.1007/s10620-018-5420-x

Reiner J, et al. Teduglutide Promotes Epithelial Tight Junction Pore Function in Murine Short Bowel Syndrome to Alleviate Intestinal Insufficiency. Dig. Dis. Sci. 2020 Feb. 1

The invention is also described by the following numbered embodiments:

1. An agonist combination for use in the treatment of a patient who has undergone surgical resection of the bowel, wherein said agonist combination is administered to said patient within about 7 days of said surgical resection, and wherein said agonist combination comprises a GLP-1 agonist and a GLP-2 agonist.

2. The agonist combination for use according to embodiment 1 wherein said GLP-1 agonist and said GLP-2 agonist are administered in the same composition.

3. The agonist combination for use according to embodiment 1 or embodiment 2 wherein said agonist combination is a dual GLP-1/GLP-2 agonist.

4. The agonist combination for use according to any one of embodiments 1 to 3 wherein said agonist combination is administered to said patient within about 6, 5, 4, 3, 2 or 1 day of said surgical resection.

4. The agonist combination for use according to any preceding embodiment wherein said agonist combination is administered to said patient within about 6, 5, 4, 3, 2 or 1 days of said surgical resection.

5. The agonist combination for use according any preceding embodiment wherein said agonist combination is administered to said patient within about 48, 44, 40, 36, 32, 28, 24, 20, 16, 12, 8 or 4 hours of said surgical resection.

6. The agonist combination for use according to any preceding embodiment wherein said patient has short bowel syndrome.

7. The agonist combination for use according to any preceding embodiment wherein said GLP-1 and/or GLP-2 agonist, or dual agonist, is a peptide.

8. The agonist combination for use according to any one of embodiments 1, 2, and 4 to 7 wherein said GLP-2 agonist is selected from native GLP-2, a GLP-2 analog such as Teduglutide, Glepaglutide, BC-GLP-2, HM15912, NB1002, Apraglutide, a synthetic GLP-2 and a GLP-2 peptibody such as SHP681.

9. The agonist combination for use according to any one of embodiments 1, 2, and 4 to 8 wherein said GLP-1 agonist is selected from native GLP-1, a GLP-1 analog, such as Liraglutide, Lixisenatide, Dulaglutide, Semaglutide, Exenatide, and a synthetic GLP-1.

10. The agonist combination for use according to any one of embodiments 3 to 9 wherein said GLP-1/GLP-2 dual agonist is a compound represented by the formula:

R¹—X*-U-R²

wherein:

R¹ is hydrogen (Hy), C₁₋₄ alkyl (e.g. methyl), acetyl, formyl, benzoyl or trifluoroacetyl; R² is NH₂ or OH;

X* is a peptide of formula I:

(I)
H-X2-EG-X5-F-X7-X8-E-X10-X11-TIL-X15-X16-X17-A-
X19-X20-X21-FI-X24-WL-X27-X28-X29-KIT-X33

wherein:

X2 is Aib or G

X5 is T or S;

X7 is T or S;

X8 is S, E or D;

X10 is L, M, V or Ψ;

X11 is A, N or S;

X15 is D or E;

X16 is G, E, A or Ψ;

X17 is Q, E, K, L or Ψ;

X19 is A, V or S;

X20 is R, K or Ψ;

X21 is D, L or E;

X24 is A, N or S;

X27 is I, Q, K, H or Y;

X28 is Q, E, A, H, Y, L, K, R or S;

X29 is H, Y, K or Q;

X33 is D or E;

U is absent or a sequence of 1-15 residues each independently selected from K, k, E, A, T, I, L and Ψ);

the molecule contains one and only one Ψ, wherein Ψ is a residue of K, k, R, Orn, Dap or Dab in which the side chain is conjugated to a substituent having the formula Z¹— or Z¹—Z²—, wherein

Z¹— is CH₃—(CH₂)₁₀₋₂₂—(CO)— or HOOC—(CH₂)₁₀₋₂₂—(CO)—; and

—Z²— is selected from —Z^{S1}—, —Z^{S1}—Z^{S2}—, —Z^{S2}—Z^{S1}, —Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, —Z^{S2}Z^{S3}Z^{S2}— wherein

Z^{S1} is isoGlu, β-Ala, isoLys, or 4-aminobutanoyl;

Z^{S2} is -(Peg3)ₘ- where m is 1, 2, or 3; and

—Z^{S3}— is a peptide sequence of 1-6 amino acid units independently selected from the group consisting of A, L, S, T, Y, Q, D, E, K, k, R, H, F and G;

and wherein at least one of X5 and X7 is T;

or a pharmaceutically acceptable salt or solvate thereof.

11. The agonist combination for use according to embodiment 10 wherein:

X2 is Aib or G;

X5 is T or S;

X7 is T or S;

X8 is S;

X10 is L or Ψ;

X11 is A or S;

X15 is D or E;

X16 is G, E, A or Ψ;

X17 is Q, E, K, L or Ψ;

X19 is A or S;

X20 is R or Ψ;

X21 is D, L or E;

X24 is A;

X27 is I, Q, K, or Y;

X28 is Q, E, A, H, Y, L, K, R or S;

X29 is H, Y or Q; and

X33 is D or E.

12. The agonist combination for use according to embodiment 10 or embodiment 11 wherein X16 is E and X17 is Q.

13. The agonist combination for use according to any one embodiments 10 to 12 wherein:

X11 is A and X15 is D;

X11 is S and X15 is E; or

X11 is A and X15 is E.

14. The agonist combination for use according to any one embodiments 10 to 13 wherein X27 is I.

15. The agonist combination for use according to any one embodiments 10 to 14 wherein X29 is H, and optionally X28 is A or X28 is E.

16. The agonist combination for use according to any one of embodiments 10 to 14 wherein X29 is Q and optionally X27 is Q.

17. The agonist combination for use according to any one of embodiments 10 to 16 wherein the residues at X27-X29 have a sequence selected from:

IQH;

IEH

IAH;

IHH;

IYH;

ILH;

IKH;

IRH;

ISH;

QQH;

YQH;

KQH;

IQQ;

IQY;

IQT; and

IAY.

18. The agonist combination for use according to embodiment 10 wherein X* is a peptide of formula II:

(II)
H-X2-EG-X5-F-X7-SELATILD-X16-X17-AAR-
X21-FIAWLI-X28-X29-KITD

wherein:

X2 is Aib or G

X5 is T or S;

X7 is T or S;

X16 is G or Ψ;

X17 is Q, E, K, L or Ψ;

X21 is D or L;

X28 is Q, E, A, H, Y, L, K, R or S;

X29 is H, Y or Q.

19. The agonist combination for use according to embodiment 10 or embodiment 18 wherein X16 is Ψ and X17 is Q, E, K or L, or X16 is G and X17 is Ψ.

20. The agonist combination for use according to any one of embodiments 10 to 19 wherein X21 is D.

21. The agonist combination for use according to embodiment 10 wherein X* is a peptide of formula III:

(III)
H[Aib]EG-X5-F-X7-SE-X10-ATILD-X16-X17-
AA-X20-X21-FIAWLI-X28-X29-KITD

wherein:

X5 is T or S;

X7 is T or S;

X10 is L or Ψ;

X16 is G, E, A or Ψ;

X17 is Q, E, K, L or Ψ;

X20 is R or Ψ;

X21 is D or L;

X28 is E, A or Q;

X29 is H, Y or Q;

and at least one of X5 and X7 is T.

22. The agonist combination for use according to embodiment 10 wherein X* is a peptide of formula IV:

(IV)
H[Aib]EG-X5-F-X7-SELATILD-X16-X17-AAR-
X21-FIAWLI-X28-X29-KITD

wherein:

X5 is T or S;

X7 is T or S;

X16 is G or Ψ;

X17 is E, K, L or Ψ;

X21 is D or L;

X28 is E or A;

X29 is H, Y or Q;

and at least one of X5 and X7 is T.

23. The agonist combination for use according to any one of embodiments 10 to 22 wherein X16 is Ψ and X17 is E, K or L.

24. The agonist combination for use according to any one of embodiments 10 to 22 wherein X16 is G and X17 is Ψ.

25. The agonist combination for use according to embodiment 23 or embodiment 24 wherein:

X21 is D and X28 is E;

X21 is D and X28 is A;

X21 is L and X28 is E;

X21 is Land X28 is A.

26. The agonist combination for use according to embodiment 10 wherein X* is a peptide of formula V:

(V)
H[Aib]EG-X5-F-X7-SELATILD-Ψ-
QAARDFIAWLI-X28-X29-KITD

wherein

X5 is T or S;

X7 is T or S;

X28 is Q, E, A, H, Y, L, K, R or S;

X29 is H, Y or Q;

and at least one of X5 and X7 is T.

27. The agonist combination for use according to any one of embodiments 10 to 26 wherein:

X5 is S and X7 is T;

X5 is T and X7 is S; or

X5 is T and X7 is T.

28. The agonist combination for use according to embodiment 27 wherein X5 is S and X7 is T, or X5 is T and X7 is T.

29. The agonist combination for use according to any one of embodiments 10 to 28 wherein Ψ is a Lys residue whose side chain is conjugated to the substituent Z¹— or Z¹—Z²—.

30. The agonist combination for use according to any one of embodiments 10 to 29 wherein Z¹— is dodecanoyl, tetradecanoyl, hexadecanoyl, octadecanoyl or eicosanoyl.

31. The agonist combination for use according to any one of embodiments 10 to 29 wherein Z¹— is:

13-carboxytridecanoyl, i.e. HOOC—(CH₂)₁₂—(CO)—;

15-carboxypentadecanoyl, i.e. HOOC—(CH₂)₁₄—(CO)—;

17-carboxyheptadecanoyl, i.e. HOOC—(CH₂)₁₆—(CO)—;

19-carboxynonadecanoyl, i.e. HOOC—(CH₂)₁₈—(CO)—; or

21-carboxyheneicosanoyl, i.e. HOOC—(CH₂)₂₀—(CO)—.

32. The agonist combination for use according to any one of embodiments 10 to 31 wherein Z² is absent.

33. The agonist combination for use according to any one of embodiments 10 to 31 wherein Z² comprises Z^{S1} alone or in combination with Z^{S2} and/or Z^{S3}.

34. The agonist combination for use according to embodiment 33 wherein:

—Z^{S1}— is isoGlu, β-Ala, isoLys, or 4-aminobutanoyl;

—Z^{S2}—, when present, is -(Peg3)ₘ- where m is 1, 2, or 3; and

—Z^{S3}— is a peptide sequence of 1-6 amino acid units independently selected from the group consisting of A, L, S, T, Y, Q, D, E, K, k, R, H, F and G, such as the peptide sequence KEK.

35. The agonist combination for use according to embodiment 33 or embodiment 34 wherein Z² has the formula —Z^{S1}—Z^{S3}—Z^{S2}—, where Z^{S1} is bonded to Z¹ and Z^{S2} is bonded to the side chain of the amino acid component of Ψ.

36. The agonist combination for use according to embodiment 35 wherein —Z²— is:

isoGlu(Peg3)₀₋₃;

β-Ala(Peg3)₀₋₃;

isoLys(Peg3)₀₋₃;

4-aminobutanoyl(Peg3)₀₋₃; or

isoGlu-KEK-(Peg3)₀₋₃.

37. The agonist combination for use according to any one of embodiments 10 to 29 wherein Z¹— or Z¹—Z²— is:

[17-carboxy-heptadecanoyl]-isoGlu;

[17-Carboxy-heptadecanoyl]-isoGlu-KEK-Peg3-;

[17-carboxy-heptadecanoyl]-isoGlu-Peg3-;

[19-Carboxy-nonadecanoyl]-isoGlu-;

[19-Carboxy-nonadecanoyl]-isoGlu-KEK-;

[19-Carboxy-nonadecanoyl]-isoGlu-KEK-Peg3-;

[19-carboxy-nonadecanoyl]-isoGlu-KEK-Peg3-Peg3-;

[19-carboxy-nonadecanoyl]-isoGlu-Peg3-Peg3-;

[19-carboxy-nonadecanoyl]-isoLys-Peg3-Peg3-Peg3-;

[Hexadecanoyl]-βAla-;

[Hexadecanoyl]-isoGlu-; or

Octadecanoyl-.

38. The agonist combination for use according to embodiment 37 wherein Ψ is:

K([17-carboxy-heptadecanoyl]-isoGlu).

K([17-Carboxy-heptadecanoyl]-isoGlu-KEK-Peg3);

K([17-carboxy-heptadecanoyl]-isoGlu-Peg3);

K([19-Carboxy-nonadecanoyl]-isoGlu);

K([19-Carboxy-nonadecanoyl]-isoGlu-KEK);

K([19-Carboxy-nonadecanoyl]-isoGlu-KEK-Peg3);

K([19-carboxy-nonadecanoyl]-isoGlu-KEK-Peg3-Peg3);

K([19-carboxy-nonadecanoyl]-isoGlu-Peg3-Peg3);

K([19-carboxy-nonadecanoyl]-isoLys-Peg3-Peg3-Peg3);

K([Hexadecanoyl]-βAla-;

K([Hexadecanoyl]-isoGlu); or

K(Octadecanoyl).

39. The agonist combination for use according to any one of embodiments 10 to 38 wherein U is 1-10 amino acids in length, 1-7 amino acids in length, 3-7 amino acids in length, 1-6 amino acids in length, or 3-6 amino acids in length.

40. The agonist combination for use according to any one of embodiments 10 to 39 wherein U includes at least one charged amino acid, e.g. two or more charged amino acids.

41. The agonist combination for use according to embodiment 40 wherein U includes at least 1 positively charged amino acid and at least one negatively charged amino acid.

42. The agonist combination for use according to embodiment 40 or embodiment 41 wherein U is a chain of alternately positively and negatively charged amino acids.

43. The agonist combination for use according to any one of embodiments 40 to 42 wherein U comprises residues selected only from K, k, E and Ψ.

44. The agonist combination for use according to embodiment 39 wherein U is K₃, K₄, K₅, K₆ , K₇, k₃, k₄, k₅, k₆ or k₇.

45. The agonist combination for use according to any one of embodiments 41 to 42 wherein U is KEK, EKEKEK, EkEkEk, AKAAEK, AKEKEK or ATILEK.

46. The agonist combination for use according to any one of embodiments 40 to 42 wherein U is K₁₋₁₄-Ψ, K₁₋₉-Ψ, K₁₋₆-Ψ, k₁₋₁₄-Ψ, k₁₋₉-Ψ, k₁₋₆-Ψ, KEΨ, EKEKEΨ, EkEkEΨ AKAAEΨ, AKEKEΨ or ATILEΨ.

47. The agonist combination for use according to any one of embodiments 10 to 38 wherein U is absent.

48. The agonist combination for use according to any one of embodiments 10 to 47 wherein R¹ is Hy and/or R² is OH.

49. The agonist combination for use according to any one of embodiments 10 to 48 wherein X* or X*-U has the sequence:

H[Aib]EGTFSSELATILDΨEAARDFIAWLIEHKITD;
H[Aib]EGSFTSELATILDΨEAARDFIAWLIEHKITD;
H[Aib]EGTFTSELATILDΨEAARDFIAWLIEHKITD;
H[Aib]EGTFSSELATILDΨKAARDFIAWLIEHKITD;
H[Aib]EGSFTSELATILDΨKAARDFIAWLIEHKITD;
H[Aib]EGTFTSELATILDΨKAARDFIAWLIEHKITD;
H[Aib]EGTFSSELATILDGΨAARDFIAWLIEHKITD;
H[Aib]EGTFSSELATILDΨEAARDFIAWLIEHKITD;
H[Aib]EGSFTSELATILDΨEAARDFIAWLIEHKITD;
H[Aib]EGTFSSELATILDΨLAARDFIAWLIEHKITD;
H[Aib]EGSFTSELATILDΨLAARDFIAWLIEHKITD;
H[Aib]EGTFTSELATILDΨLAARDFIAWLIEHKITD;
H[Aib]EGTFSSELATILDΨLAARDFIAWLIAHKITD;
H[Aib]EGSFTSELATILDΨLAARDFIAWLIAHKITD;
H[Aib]EGTFTSELATILDΨLAARDFIAWLIAHKITD;
H[Aib]EGTFTSELATILDΨEAARLFIAWLIEHKITD;
H[Aib]EGTFSSELATILDΨQAARDFIAWLIQHKITD;
H[Aib]EGSFTSELATILDΨQAARDFIAWLIQHKITD;
H[Aib]EGTFTSELATILD+QAARDFIAWLIQHKITD;
H[Aib]EGTFSSELATILDΨQAARDFIAWLIEHKITD;
H[Aib]EGTFSSELATILDΨQAARDFIAWLIAHKITD;
H[Aib]EGSFTSELATILDΨQAARDFIAWLIAHKITD;
H[Aib]EGTFTSELATILDΨQAARDFIAWLIAHKITD;
H[Aib]EGSFTSELATILDΨQAARDFIAWLIEHKITD;
H[Aib]EGTFTSELATILDΨQAARDFIAWLIEHKITD;
H[Aib]EGSFTSELATILDΨQAARDFIAWLIHHKITD;
H[Aib]EGSFTSELATILDΨQAARDFIAWLIYHKITD;
H[Aib]EGSFTSELATILDΨQAARDFIAWLILHKITD;
H[Aib]EGSFTSELATILDΨQAARDFIAWLIKHKITD;
H[Aib]EGSFTSELATILDΨQAARDFIAWLIRHKITD;
H[Aib]EGSFTSELATILDΨQAARDFIAWLISHKITD
H[Aib]EGSFTSELATILDΨQAARDFIAWLQQHKITD;
H[Aib]EGSFTSELATILDΨQAARDFIAWLYQHKITD;
H[Aib]EGSFTSELATILDΨQAARDFIAWLKQHKITD;
H[Aib]EGSFTSELATILDΨQAARDFIAWLIQQKITD;
H[Aib]EGSFTSELATILDΨQAARDFIAWLIQYKITD;
H[Aib]EGTFSSELSTILEΨQASREFIAWLIAYKITE;
H[Aib]EGTFSSELATILDEQAARDFIAWLIAHKITDkkkkkΨ;
H[Aib]EGTFTSELATILDEQAARDFIAWLIAHKITDkkkkkΨ;
H[Aib]EGSFTSELATILDEQAARDFIAWLIEHKITDkkkkkΨ;
H[Aib]EGSFTSEΨATILDEQAARDFIAWLIEHKITD;
H[Aib]EGSFTSELATILEGΨAARDFIAWLIEHKITD;
H[Aib]EGSFTSELATILDEQAAΨDFIAWLIEHKITD;
H[Aib]EGTFTSELATILDEQAAVDFIAWLIEHKITD;
H[AIb]EGTFTSEWATILDEQAARDFIAWLIEHKITD;
H[AIb]EGSFTSELATILDAWAARDFIAWLIEHKITD;
or
H[Aib]EGSFTSELATILDAKAAWDFIAWLIEHKITD.

50. The agonist combination for use according to embodiment 49 wherein X* or X*-U has the sequence:

H[Aib]EGTFSSELATILD[K*]EAARDFIAWLIEHKITD;
H[Aib]EGSFTSELATILD[K*]EAARDFIAWLIEHKITD;
H[Aib]EGTFTSELATILD[K*]EAARDFIAWLIEHKITD;
H[Aib]EGTFSSELATILD[K*]KAARDFIAWLIEHKITD;
H[Aib]EGSFTSELATILD[K*]KAARDFIAWLIEHKITD;
H[Aib]EGTFTSELATILD[K*]KAARDFIAWLIEHKITD;
H[Aib]EGTFSSELATILDG[K*]AARDFIAWLIEHKITD;
H[Aib]EGSFTSELATILDG[K*]AARDFIAWLIEHKITD;
H[Aib]EGTFTSELATILDG[K*]AARDFIAWLIEHKITD;
H[Aib]EGTFSSELATILD[K*]LAARDFIAWLIEHKITD;
H[Aib]EGSFTSELATILD[K*]LAARDFIAWLIEHKITD;
H[Aib]EGTFTSELATILD[K*]LAARDFIAWLIEHKITD;
H[Aib]EGTFSSELATILD[K*]LAARDFIAWLIAHKITD;
H[Aib]EGSFTSELATILD[K*]LAARDFIAWLIAHKITD;
H[Aib]EGTFTSELATILD[K*]LAARDFIAWLIAHKITD;
H[Aib]EGTFTSELATILD[K*]EAARLFIAWLIEHKITD;
H[Aib]EGTFSSELATILD[K*]QAARDFIAWLIQHKITD;
H[Aib]EGSFTSELATILD[K*]QAARDFIAWLIQHKITD;
H[Aib]EGTFTSELATILD[K*]QAARDFIAWLIQHKITD;
H[Aib]EGTFSSELATILD[K*]QAARDFIAWLIEHKITD;
H[Aib]EGTFSSELATILD[K*]QAARDFIAWLIAHKITD;
H[Aib]EGSFTSELATILD[K*]QAARDFIAWLIAHKITD;
H[Aib]EGTFTSELATILD[K*]QAARDFIAWLIAHKITD;
H[Aib]EGSFTSELATILD[K*]QAARDFIAWLIEHKITD;
H[Aib]EGTFTSELATILD[K*]QAARDFIAWLIEHKITD;
H[Aib]EGSFTSELATILD[K*]QAARDFIAWLIHHKITD;
H[Aib]EGSFTSELATILD[K*]QAARDFIAWLIYHKITD;
H[Aib]EGSFTSELATILD[K*]QAARDFIAWLILHKITD;
H[Aib]EGSFTSELATILD[K*]QAARDFIAWLIKHKITD;
H[Aib]EGSFTSELATILD[K*]QAARDFIAWLIRHKITD;
H[Aib]EGSFTSELATILD[K*]QAARDFIAWLISHKITD;
H[Aib]EGSFTSELATILD[K*]QAARDFIAWLQQHKITD;
H[Aib]EGSFTSELATILD[K*]QAARDFIAWLYQHKITD;
H[Aib]EGSFTSELATILD[K*]QAARDFIAWLKQHKITD;
H[Aib]EGSFTSELATILD[K*]QAARDFIAWLIQQKITD;
H[Aib]EGSFTSELATILD[K*]QAARDFIAWLIQYKITD;
H[Aib]EGTFSSELSTILE[K*]QASREFIAWLIAYKITE;
H[Aib]EGTFSSELATILDEQAARDFIAWLIAHKITDkkkkk[k*]
H[Aib]EGTFTSELATILDEQAARDFIAWLIAHKITDkkkkk[k*]
H[Aib]EGSFTSELATILDEQAARDFIAWLIEHKITDkkkkk[k*]
H[Aib]EGSFTSE[K*]ATILDEQAARDFIAWLIEHKITD;
H[Aib]EGSFTSELATILEG[K*]AARDFIAWLIEHKITD;
H[Aib]EGSFTSELATILDEQAA[K*]DFIAWLIEHKITD;
H[Aib]EGTFTSELATILDEQAA[K*]DFIAWLIEHKITD;
H[Aib]EGTFTSE[K*]ATILDEQAARDFIAWLIEHKITD;
H[Aib]EGSFTSELATI LDA[K*]AARDFIAWLIEHKITD;
or
H[Aib]EGSFTSELATILDAKAA[K*]DFIAWLIEHKITD;

wherein K* or k* indicates an L or D lysine residue respectively in which the side chain is conjugated to the substituent Z¹— or Z¹Z²—.

51. The agonist combination for use according to embodiment 50 wherein X* or X*-U has the sequence:

H[Aib]EGTFSSELATILD[K([17-carboxy-
heptadecanoyl]-isoGlu)]EAARDFIAWLIEHKITD;
H[Aib]EGSFTSELATILD[K([17-carboxy-
heptadecanoyl]-isoGlu)]EAARDFIAWLIEHKITD;
H[Aib]EGTFTSELATILD[K([17-carboxy-
heptadecanoyl]-isoGlu)]EAARDFIAWLIEHKITD;
H[Aib]EGTFSSELATILD[K([17-carboxy-
heptadecanoyl]-isoGlu)]KAARDFIAWLIEHKITD;
H[Aib]EGSFTSELATILD[K([17-carboxy-
heptadecanoyl]-isoGlu)]KAARDFIAWLIEHKITD;
H[Aib]EGTFTSELATILD[K([17-carboxy-
heptadecanoyl]-isoGlu)]KAARDFIAWLIEHKITD;
H[Aib]EGTFSSELATILDG[K([17-carboxy-
heptadecanoyl]-isoGlu)]AARDFIAWLIEHKITD;
H[Aib]EGSFTSELATILDG[K([17-carboxy-
heptadecanoyl]-isoGlu)]AARDFIAWLIEHKITD;
H[Aib]EGTFTSELATILDG[K([17-carboxy-
heptadecanoyl]-isoGlu)]AARDFIAWLIEHKITD;
H[Aib]EGTFSSELATILD[K([17-carboxy-
heptadecanoyl]-isoGlu)]LAARDFIAWLIEHKITD;
H[Aib]EGSFTSELATILD[K([17-carboxy-
heptadecanoyl]-isoGlu)]LAARDFIAWLIEHKITD;
H[Aib]EGTFTSELATILD[K([17-carboxy-
heptadecanoyl]-isoGlu)]LAARDFIAWLIEHKITD;
H[Aib]EGTFSSELATILD[K([17-carboxy-
heptadecanoyl]-isoGlu)]LAARDFIAWLIAHKITD;
H[Aib]EGSFTSELATILD[K([17-carboxy-
heptadecanoyl]-isoGlu)]LAARDFIAWLIAHKITD;
H[Aib]EGTFTSELATILD[K([17-carboxy-
heptadecanoyl]-isoGlu)]LAARDFIAWLIAHKITD;
H[Aib]EGTFTSELATILD[K([17-carboxy-
heptadecanoyl]-isoGlu)]EAARLFIAWLIEHKITD;
H[Aib]EGTFSSELATILD[K([17-carboxy-
heptadecanoyl]-isoGlu)]QAARDFIAWLIQHKITD;
H[Aib]EGSFTSELATILD[K([17-carboxy-
heptadecanoyl]-isoGlu)]QAARDFIAWLIQHKITD;
H[Aib]EGTFTSELATILD[K([17-carboxy-
heptadecanoyl]-isoGlu)]QAARDFIAWLIQHKITD;
H[Aib]EGTFSSELATILD[K([17-carboxy-
heptadecanoyl]-isoGlu)]QAARDFIAWLIEHKITD;
H[Aib]EGTFSSELATILD[K([17-carboxy-
heptadecanoyl]-isoGlu)]QAARDFIAWLIAHKITD;
H[Aib]EGSFTSELATILD[K([17-carboxy-
heptadecanoyl]-isoGlu)]QAARDFIAWLIAHKITD;
H[Aib]EGTFTSELATILD[K([17-carboxy-
heptadecanoyl]-isoGlu)]QAARDFIAWLIAHKITD;
H[Aib]EGSFTSELATILD[K([17-carboxy-
heptadecanoyl]-isoGlu)]QAARDFIAWLIEHKITD;
H[Aib]EGTFTSELATILD[K([17-carboxy-
heptadecanoyl]-isoGlu)]QAARDFIAWLIEHKITD;
H[Aib]EGSFTSELATILD[K([17-carboxy-
heptadecanoyl]-isoGlu)]QAARDFIAWLIHHKITD;
H[Aib]EGSFTSELATILD[K([17-carboxy-
heptadecanoyl]-isoGlu)]QAARDFIAWLIYHKITD;
H[Aib]EGSFTSELATILD[K([17-carboxy-
heptadecanoyl]-isoGlu)]QAARDFIAWLILHKITD;
H[Aib]EGSFTSELATILD[K([17-carboxy-
heptadecanoyl]-isoGlu)]QAARDFIAWLIKHKITD;
H[Aib]EGSFTSELATILD[K([17-carboxy-
heptadecanoyl]-isoGlu)]QAARDFIAWLIRHKITD;
H[Aib]EGSFTSELATILD[K([17-carboxy-
heptadecanoyl]-isoGlu)]QAARDFIAWLISHKITD;
H[Aib]EGSFTSELATILD[K([Hexadecanoyl]-
pAla)]QAARDFIAWLQQHKITD;
H[Aib]EGSFTSELATILD[K([17-carboxy-
heptadecanoyl]iso-Glu-Peg3)]
QAARDFIAWLYQHKITD;
H[Aib]EGSFTSELATILD[K([19-carboxy-
nonadecanoyl]iso-Glu-Peg3-Peg3)]
QAARDFIAWLKQHKITD;
H[Aib]EGSFTSELATILD[K([19-carboxy-
nonadecanoyl]iso-Lys-Peg3-Peg3-Peg3)]
QAARDFIAWLIQQKITD;
H[Aib]EGSFTSELATILD[K(Octadecanoyl)]
QAARDFIAWLIQYKITD;
H[Aib]EGTFSSELSTILE[K(Hexadecanoyl-isoGlu)]
QASREFIAWLIAYKITE;
H[Aib]EGTFSSELATILDEQAARDFIAWLIAHKITDkkkkkk
([17-carboxy-
Heptadecanoyl]-isoGlu)];
H[Aib]EGTFTSELATILDEQAARDFIAWLIAHKITDkkkkkk
([17-carboxy-Heptadecanoyl]-isoGlu)];
H[Aib]EGSFTSELATILDEQAARDFIAWLIEHKITDkkkkkk
([17-carboxy-Heptadecanoyl]-isoGlu)];
H[Aib]EGTFTSELATILD[K([19-Carboxy-
nonadecanoyl]-isoGlu)]QAARDFIAWLIQHKITD;
H[Aib]EGSFTSE[K([19-carboxy-
nonadecanoyl]iso-Glu-Peg3-Peg3)]
ATILDEQAARDFIAWLIEHKITD;
H[Aib]EGSFTSELATILD[K([19-carboxy-
nonadecanoyl]iso-Glu-Peg3-Peg3)]
KAARDFIAWLIEHKITD;
H[Aib]EGSFTSELATILEG[K([19-carboxy-
nonadecanoyl]iso-Glu-Peg3-Peg3)]
AARDFIAWLIEHKITD;
H[Aib]EGSFTSELATILDEQAA[K([19-carboxy-
nonadecanoyl]iso-Glu-Peg3-Peg3)]
DFIAWLIEHKITD;
H[Aib]EGTFTSELATILDEQAA[K([19-carboxy-
nonadecanoyl]iso-Glu-Peg3-Peg3)]
DFIAWLIEHKITD;
H[AIb]EGTFSSELATILD[K([17-Carboxy-
heptadecanoyl]-isoGlu-KEK-Peg3)]
QAARDFIAWLIQHKITD;
H[Aib]EGTFSSELATILD[K([19-Carboxy-
nonadecanoyl]-isoGlu-KEK-Peg3)]
QAARDFIAWLIQHKITD;
H[Aib]EGTFSSELATILD[K([17-Carboxy-
heptadecanoyl]-isoGlu-KEK-Peg3)]
QAARDFIAWLIEHKITD;
H[Aib]EGTFSSELATILD[K([19-Carboxy-
nonadecanoyl]-isoGlu-KEK-Peg3)]
QAARDFIAWLIEHKITD;
H[Aib]EGTFTSELATILD[K([19-Carboxy-
nonadecanoyl]-isoGlu-KEK)]
QAARDFIAWLIQHKITD;
H[Aib]EGTFTSELATILD[K([19-Carboxy-
nonadecanoyl]-isoGlu-KEK-Peg3)]
QAARDFIAWLIQHKITD;
H[Aib]EGSFTSE[K([19-Carboxy-
nonadecanoyl]-isoGlu-KEK-Peg3)]
ATILDEQAARDFIAWLIEHKITD;
H[Aib]EGTFTSE[K([19-Carboxy-
nonadecanoyl]-isoGlu-KEK-Peg3)]
ATILDEQAARDFIAWLIEHKITD;
H[Aib]EGSFTSE[K([19-carboxy-
nonadecanoyl]iso-Glu-KEK-Peg3-Peg3)]
ATILDEQAARDFIAWLIEHKITD;
H[Aib]EGTFTSELATILD[K([19-Carboxy-
nonadecanoyl]-isoGlu-KEK-Peg3)]
QAARDFIAWLIEHKITD;
H[Aib]EGSFTSELATILD[K([19-Carboxy-
nonadecanoyl]-isoGlu-KEK-Peg3)]
QAARDFIAWLIEHKITD;
H[Aib]EGSFTSELATILD[K([19-Carboxy-
nonadecanoyl]-isoGlu-KEK-Peg3)]
QAARDFIAWLIAHKITD;
H[Aib]EGSFTSELATILD[K([19-Carboxy-
nonadecanoyl]-isoGlu-KEK-Peg3)]
KAARDFIAWLIEHKITD;
H[Aib]EGSFTSELATILD[K([19-carboxy-
nonadecanoyl]iso-Glu-KEK-Peg3-Peg3)]
QAARDFIAWLIEHKITD;
H[Aib]EGSFTSELATILEG[K([19-Carboxy-
nonadecanoyl]-isoGlu-KEK-Peg3)]
AARDFIAWLIEHKITD;
H[Aib]EGSFTSELATILDA[K([19-Carboxy-
nonadecanoyl]-isoGlu-KEK-Peg3)]
AARDFIAWLIEHKITD;
H[Aib]EGSFTSELATILDA[K([19-carboxy-
nonadecanoyl]iso-Glu-KEK-Peg3-Peg3)]
AARDFIAWLIEHKITD;
H[Aib]EGSFTSELATILDEQAA[K([19-Carboxy-
nonadecanoyl]-isoGlu-KEK-Peg3)]
DFIAWLIEHKITD;
H[Aib]EGTFTSELATILDEQAA[K([19-Carboxy-
nonadecanoyl]-isoGlu-KEK-Peg3)]
DFIAWLIEHKITD;
H[Aib]EGSFTSELATILDEQAA[K([19-carboxy-
nonadecanoyl]iso-Glu-KEK-Peg3-Peg3)]
DFIAWLIEHKITD;
H[Aib]EGTFTSELATILDEQAA[K([19-carboxy-
nonadecanoyl]iso-Glu-KEK-Peg3-Peg3)]
DFIAWLIEHKITD;
or
H[Aib]EGSFTSELATILDAKAA[K([19-Carboxy-
nonadecanoyl]-isoGlu-KEK-Peg3)]
DFIAWLIEHKITD.

52. The agonist combination for use according to embodiment 51 which is:

Hy-H[Aib]EGTFSSELATILD[K([17-
carboxy-heptadecanoyl]-
isoGlu)]EAARDFIAWLIEHKITD-OH (Compound 1);
Hy-H[Aib]EGSFTSELATILD[K([17-
carboxy-heptadecanoyl]-
isoGlu)]EAARDFIAWLIEHKITD-OH (Compound 2);
Hy-H[Aib]EGTFTSELATILD[K([17-
carboxy-heptadecanoyl]-
isoGlu)]EAARDFIAWLIEHKITD-OH (Compound 3);
Hy-H[Aib]EGTFSSELATILD[K([17-
carboxy-heptadecanoyl]-
isoGlu)]KAARDFIAWLIEHKITD-OH (Compound 4);
Hy-H[Aib]EGSFTSELATILD[K([17-
carboxy-heptadecanoyl]-
isoGlu)]KAARDFIAWLIEHKITD-OH (Compound 5);
Hy-H[Aib]EGTFTSELATILD[K([17-
carboxy-heptadecanoyl]-
isoGlu)]KAARDFIAWLIEHKITD-OH (Compound 6);
Hy-H[Aib]EGTFSSELATILDG[K([17-
carboxy-heptadecanoyl]-
isoGlu)]AARDFIAWLIEHKITD-OH (Compound 7);
Hy-H[Aib]EGSFTSELATILDG[K([17-
carboxy-heptadecanoyl]-
isoGlu)]AARDFIAWLIEHKITD-OH (Compound 8);
Hy-H[Aib]EGTFTSELATILDG[K([17-
carboxy-heptadecanoyl]-
isoGlu)]AARDFIAWLIEHKITD-OH (Compound 9);
Hy-H[Aib]EGTFSSELATILD[K([17-
carboxy-heptadecanoyl]-
isoGlu)]LAARDFIAWLIEHKITD-OH (Compound 10);
Hy-H[Aib]EGSFTSELATILD[K([17-
carboxy-heptadecanoyl]-
isoGlu)]LAARDFIAWLIEHKITD-OH (Compound 11);
Hy-H[Aib]EGTFTSELATILD[K([17-
carboxy-heptadecanoyl]-
isoGlu)]LAARDFIAWLIEHKITD-OH (Compound 12);
Hy-H[Aib]EGTFSSELATILD[K([17-
carboxy-heptadecanoyl]-
isoGlu)]LAARDFIAWLIAHKITD-OH (Compound 13);
Hy-H[Aib]EGSFTSELATILD[K([17-
carboxy-heptadecanoyl]-
isoGlu)]LAARDFIAWLIAHKITD-OH (Compound 14);
Hy-H[Aib]EGTFTSELATILD[K([17-
carboxy-heptadecanoyl]-
isoGlu)]LAARDFIAWLIAHKITD-OH (Compound 15);
Hy-H[Aib]EGTFTSELATILD[K([17-
carboxy-heptadecanoyl]-
isoGlu)]EAARLFIAWLIEHKITD-OH (Compound 16);
Hy-H[Aib]EGTFSSELATILD[K([17-
carboxy-heptadecanoyl]-
isoGlu)]QAARDFIAWLIQHKITD-OH (Compound 17);
Hy-H[Aib]EGSFTSELATILD[K([17-
carboxy-heptadecanoyl]-
isoGlu)]QAARDFIAWLIQHKITD-OH (Compound 18);
Hy-H[Aib]EGTFTSELATILD[K([17-
carboxy-heptadecanoyl]-
isoGlu)]QAARDFIAWLIQHKITD-OH (Compound 19);
Hy-H[Aib]EGTFSSELATILD[K([17-
carboxy-heptadecanoyl]-
isoGlu)]QAARDFIAWLIEHKITD-OH (Compound 20);
Hy-H[Aib]EGTFSSELATILD[K([17-
carboxy-heptadecanoyl]-
isoGlu)]QAARDFIAWLIAHKITD-OH (Compound 21);
Hy-H[Aib]EGSFTSELATILD[K([17-
carboxy-heptadecanoyl]-
isoGlu)]QAARDFIAWLIAHKITD-OH (Compound 22);
Hy-H[Aib]EGTFTSELATILD[K([17-
carboxy-heptadecanoyl]-
isoGlu)]QAARDFIAWLIAHKITD-OH (Compound 23);
Hy-H[Aib]EGSFTSELATILD[K([17-
carboxy-heptadecanoyl]-
isoGlu)]QAARDFIAWLIEHKITD-OH (Compound 24);
Hy-H[Aib]EGTFTSELATILD[K([17-
carboxy-heptadecanoyl]-
isoGlu)]QAARDFIAWLIEHKITD-OH (Compound 25);
Hy-H[Aib]EGSFTSELATILD[K([17-
carboxy-heptadecanoyl]-
isoGlu)]QAARDFIAWLIHHKITD-OH (Compound 26);
Hy-H[Aib]EGSFTSELATILD[K([17-
carboxy-heptadecanoyl]-
isoGlu)]QAARDFIAWLIYHKITD-OH (Compound 27);
Hy-H[Aib]EGSFTSELATILD[K([17-
carboxy-heptadecanoyl]-
isoGlu)]QAARDFIAWLILHKITD-OH (Compound 28);
Hy-H[Aib]EGSFTSELATILD[K([17-
carboxy-heptadecanoyl]-
isoGlu)]QAARDFIAWLIKHKITD-OH (Compound 29);
Hy-H[Aib]EGSFTSELATILD[K([17-
carboxy-heptadecanoyl]-
isoGlu)]QAARDFIAWLIRHKITD-OH (Compound 30);
Hy-H[Aib]EGSFTSELATILD[K([17-
carboxy-heptadecanoyl]-
isoGlu)]QAARDFIAWLISHKITD-OH (Compound 31).
Hy-H[Aib]EGSFTSELATILD[K([Hexadecanoyl]-
pAla)]QAARDFIAWLQQHKITD-OH
(Compound 32);
Hy-H[Aib]EGSFTSELATILD[K([17-
carboxy-heptadecanoyl]iso-Glu-
Peg3)]QAARDFIAWLYQHKITD-OH (Compound 33);
Hy-H[Aib]EGSFTSELATILD[K([19-
carboxy-nonadecanoyl]iso-Glu-Peg3-
Peg3)]QAARDFIAWLKQHKITD-OH (Compound 34);
Hy-H[Aib]EGSFTSELATILD[K([19-
carboxy-nonadecanoyl]iso-Lys-Peg3-Peg3-
Peg3)]QAARDFIAWLIQQKITD-OH (Compound 35);
Hy-H[Aib]EGSFTSELATILD[K(Octadecanoyl)]
QAARDFIAWLIQYKITD-OH
(Compound 36);
Hy-H[Aib]EGTFSSELSTILE[K(Hexadecanoyl-
isoGlu)]QASREFIAWLIAYKITE-OH
(Compound 37);
Hy-H[Aib]EGTFSSELATILDEQAARDFIAWLIAH
KITDkkkkkk([17-carboxy-
Heptadecanoyl]-isoGlu)]-[NH2] (Compound 38);
Hy-H[Aib]EGTFTSELATILDEQAARDFIAWLIAHK
ITDkkkkkk([17-carboxy-
Heptadecanoyl]-isoGlu)]-[NH2] (Compound 39);
Hy-H[Aib]EGSFTSELATILDEQAARDFIAW
LIEHKITDkkkkkk([17-carboxy-
Heptadecanoyl]-isoGlu)]-[NH2] (Compound 40);
Hy-H[Aib]EGTFTSELATILD[K([19-
Carboxy-nonadecanoyl]-
isoGlu)]QAARDFIAWLIQHKITD-OH (Compound 41);
Hy-H[Aib]EGSFTSE[K([19-
carboxy-nonadecanoyl]iso-Glu-Peg3-
Peg3)]ATILDEQAARDFIAWLIEHKITD-OH
(Compound 42);
Hy-H[Aib]EGSFTSELATILD[K([19-
carboxy-nonadecanoyl]iso-Glu-Peg3-
Peg3)]KAARDFIAWLIEHKITD-OH (Compound 43);
Hy-H[Aib]EGSFTSELATILEG[K([19-
carboxy-nonadecanoyl]iso-Glu-Peg3-
Peg3)]AARDFIAWLIEHKITD-OH (Compound 44);
Hy-H[Aib]EGSFTSELATILDEQAA[K([19-
carboxy-nonadecanoyl]iso-Glu-Peg3-
Peg3)]DFIAWLIEHKITD-OH (Compound 45);
Hy-H[Aib]EGTFTSELATILDEQAA[K([19-
carboxy-nonadecanoyl]iso-Glu-Peg3-
Peg3)]DFIAWLIEHKITD-OH (Compound 46).
Hy-H[Aib]EGTFSSELATILD[K([17-
Carboxy-heptadecanoyl]-isoGlu-KEK-
Peg3)]QAARDFIAWLIQHKITD-OH (Compound 47);
Hy-H[Aib]EGTFSSELATILD[K([19-
Carboxy-nonadecanoyl]-isoGlu-KEK-
Peg3)]QAARDFIAWLIQHKITD-OH (Compound 48);
Hy-H[Aib]EGTFSSELATILD[K([17-
Carboxy-heptadecanoyl]-isoGlu-KEK-
Peg3)]QAARDFIAWLIEHKITD-OH (Compound 49);
Hy-H[Aib]EGTFSSELATILD[K([19-
Carboxy-nonadecanoyl]-isoGlu-KEK-
Peg3)]QAARDFIAWLIEHKITD-OH (Compound 50);
Hy-H[Aib]EGTFTSELATILD[K([19-
Carboxy-nonadecanoyl]-isoGlu-
KEK)]QAARDFIAWLIQHKITD-OH (Compound 51);
Hy-H[Aib]EGTFTSELATILD[K([19-
Carboxy-nonadecanoyl]-isoGlu-KEK-
Peg3)]QAARDFIAWLIQHKITD-OH (Compound 52);
Hy-H[Aib]EGSFTSE[K([19-
Carboxy-nonadecanoyl]-isoGlu-KEK-
Peg3)]ATILDEQAARDFIAWLIEHKITD-OH
(Compound 53);
Hy-H[Aib]EGTFTSE[K([19-
Carboxy-nonadecanoyl]-isoGlu-KEK-
Peg3)]ATILDEQAARDFIAWLIEHKITD-OH
(Compound 54);
Hy-H[Aib]EGSFTSE[K([19-
carboxy-nonadecanoyl]iso-Glu-KEK-Peg3-
Peg3)]ATILDEQAARDFIAWLIEHKITD-OH
(Compound 55);
Hy-H[Aib]EGTFTSELATILD[K([19-
Carboxy-nonadecanoyl]-isoGlu-KEK-
Peg3)]QAARDFIAWLIEHKITD-OH (Compound 56);
Hy-H[Aib]EGSFTSELATILD[K([19-
Carboxy-nonadecanoyl]-isoGlu-KEK-
Peg3)]QAARDFIAWLIEHKITD-OH (Compound 57);
Hy-H[Aib]EGSFTSELATILD[K([19-
Carboxy-nonadecanoyl]-isoGlu-KEK-
Peg3)]QAARDFIAWLIAHKITD-OH (Compound 58);
Hy-H[Aib]EGSFTSELATILD[K([19-
Carboxy-nonadecanoyl]-isoGlu-KEK-
Peg3)]KAARDFIAWLIEHKITD-OH (Compound 59);
Hy-H[Aib]EGSFTSELATILD[K([19-
carboxy-nonadecanoyl]iso-Glu-KEK-Peg3-
Peg3)]QAARDFIAWLIEHKITD-OH (Compound 60);
Hy-H[Aib]EGSFTSELATILEG[K([19-
Carboxy-nonadecanoyl]-isoGlu-KEK-
Peg3)]AARDFIAWLIEHKITD-OH (Compound 61);
Hy-H[Aib]EGSFTSELATILDA[K([19-
Carboxy-nonadecanoyl]-isoGlu-KEK-
Peg3)]AARDFIAWLIEHKITD-OH (Compound 62);
Hy-H[Aib]EGSFTSELATILDA[K([19-
carboxy-nonadecanoyl]iso-Glu-KEK-Peg3-
Peg3)]AARDFIAWLIEHKITD-OH (Compound 63);
Hy-H[Aib]EGSFTSELATILDEQAA[K([19-
Carboxy-nonadecanoyl]-isoGlu-KEK-
Peg3)]DFIAWLIEHKITD-OH (Compound 64);
Hy-H[Aib]EGTFTSELATILDEQAA[K([19-
Carboxy-nonadecanoyl]-isoGlu-KEK-
Peg3)]DFIAWLIEHKITD-OH (Compound 65);
Hy-H[Aib]EGSFTSELATILDEQAA[K([19-
carboxy-nonadecanoyl]iso-Glu-KEK-Peg3-
Peg3)]DFIAWLIEHKITD-OH (Compound 66);
Hy-H[Aib]EGTFTSELATILDEQAA[K([19-
carboxy-nonadecanoyl]iso-Glu-KEK-Peg3-
Peg3)]DFIAWLIEHKITD-OH (Compound 67);
or
Hy-H[Aib]EGSFTSELATILDAKAA[K([19-
Carboxy-nonadecanoyl]-isoGlu-KEK-
Peg3)]DFIAWLIEHKITD-OH (Compound 68).

53. The agonist combination for use according to embodiment 3 wherein said GLP-1/GLP-2 dual agonist is a compound represented by the formula:

R¹—X*-U-R²

wherein:

R¹ is hydrogen (Hy), C₁₋₄ alkyl (e.g. methyl), acetyl, formyl, benzoyl or trifluoroacetyl;

R² is NH₂ or OH;

X* is a peptide of formula I:

(I)
H-X2-EG-X5-F-X7-X8-E-X10-X11-TIL-X15-X16-X17-A-
X19-X20-X21-FI-X24-WL-X27-X28-X29-KIT-X33

wherein

X2 is Aib or G;

X5 is S or T;

X7 is S or T;

X8 is S, E or D;

X10 is L, M or V;

X11 is A, N or S;

X15 is D or E

X16 is E, A or G;

X17 is Q, E, L or K;

X19 is A, V or S;

X20 is R or K;

X21 is D, L or E;

X24 is A, N or S;

X27 is I, Y, Q, H or K;

X28 is A, E, H, Y, L, K, Q, R or S;

X29 is H, Y, K or Q;

X33 is D or E;

U is absent or a sequence of 1-15 residues, each independently selected from K and k;

and wherein at least one of X5 and X7 is T;

or a pharmaceutically acceptable salt or solvate thereof.

54. The agonist combination for use according to any one of embodiments 3 to 53, or a pharmaceutically acceptable salt or solvate thereof, wherein said agonist or dual agonist is in admixture with a carrier.

55. The agonist combination for use according to any preceding embodiment wherein said agonist combination is in a composition, preferably a pharmaceutical composition.

56 The agonist combination for use according to any preceding embodiment wherein said agonist combination is in admixture with a pharmaceutically acceptable carrier, excipient or vehicle.

57. The agonist combination for use according to any preceding embodiment wherein said agonist combination is administered at a dose of about 0.1 pmol/kg to 500 μmol/kg body weight, preferably about 50 pmol/kg to 500 nmol/kg.

58. The agonist combination for use according to any preceding embodiment wherein said agonist combination is administered for 1, 2, 3, 4, 5, 6, or 7 or more days after surgery.

59. The agonist combination for use according to embodiment 58 wherein said agonist combination is administered for 1, 2, 3, 4 or more weeks, 1, 2, 3, 4, 5, 6, 7, 8, 9, 10, 11 or 12 months, or about 1 year after surgery.

60. The agonist combination for use according to any preceding embodiment which is administered in an amount effective to achieve a blood concentration of at least 0.1 nmol/L of said dual agonist in said patient.

61. The agonist combination for use according to any preceding embodiment wherein said agonist combination is also administered to said patient prior to said surgical resection.

62. The agonist combination for use according to any preceding embodiment, wherein at least about 0.5, 0.6, 0.7. 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, or 20.0% of the bowel of said patient is removed.

63. The agonist combination for use according to embodiment 62, wherein at least about 30.0, 40.0, 50.0, 60.0, 70.0 or 80.0% of the bowel of said patient is removed.

64. The agonist combination for use according to any preceding embodiment, wherein said patient receives enteral nutrition within 48 hours of said surgical resection.

65. The agonist combination for use according to embodiment 64, wherein said patient receives enteral nutrition within 24 hours of said surgical resection.

66. The agonist combination for use according to embodiment 64 or embodiment 65, wherein said enteral nutrition constitutes less than about 80% of the recommended daily intake.

67. The agonist combination for use according to any one of embodiments 64 to 66, wherein said enteral nutrition provides at least about 100 kcal per day.

68. The agonist combination for use according to any preceding embodiment, wherein said patient does not receive enteral nutrition prior to said surgical resection.

69. The agonist combination for use according to any preceding embodiment, wherein said patient has short bowel syndrome secondary to one or more of digestion disorders, malabsorption syndromes, short-gut syndrome, cul-de-sac syndrome, inflammatory bowel disease, celiac sprue (for example arising from gluten induced enteropathy or celiac disease), tropical sprue, hypogammaglobulinemic sprue, enteritis, ulcerative colitis, small intestine damage Crohn's disease, mesenteric infarction, volvulus, multiple strictures due to adhesions or radiation, vascular ischemia, necrotising enteral colitis (NEC), intestinal malformations, intestinal atresia, bowel cancer, surgical complications, acute injury, for example a stab injury.

70. The agonist combination for use according to any preceding embodiment, wherein the agonist combination is effective to increase villus growth, increase intestinal length, increase cell growth (trophic effect), increase weight gain, increase efficiency of nutrient uptake, increase mucosal height, and/or increase intestinal weight.

1. An agonist combination for use in the treatment of a patient who has undergone surgical resection of the bowel, wherein said agonist combination is administered to said patient within about 7 days of said surgical resection, and wherein said agonist combination comprises a GLP-1 agonist and a GLP-2 agonist.

2. The agonist combination for use according to claim 1 wherein said GLP-1 agonist and said GLP-2 agonist are administered in the same composition.

3. The agonist combination for use according to claim 1 or claim 2 wherein said agonist combination is a dual GLP-1/GLP-2 agonist.

4. The agonist combination for use according to any one of claims 1 to 3 wherein said agonist combination is administered to said patient within about 6, 5, 4, 3, 2 or 1 day of said surgical resection.

5. The agonist combination for use according any preceding claim wherein said agonist combination is administered to said patient within about 48, 44, 40, 36, 32, 28, 24, 20, 16, 12, 8 or 4 hours of said surgical resection.

6. The agonist combination for use according to any preceding claim wherein said patient has short bowel syndrome.

7. The agonist combination for use according to any preceding claim wherein said GLP-1 and/or GLP-2 agonist, or dual agonist, is a peptide.

8. The agonist combination for use according to any one of embodiments 1, 2, and 4 to 7 wherein said GLP-2 agonist is selected from native GLP-2, a GLP-2 analog such as Teduglutide, Glepaglutide, BC-GLP-2, HM15912, NB1002, Apraglutide, a synthetic GLP-2 and a GLP-2 peptibody such as SHP681.

9. The agonist combination for use according to any one of embodiments 1, 2, and 4 to 8 wherein said GLP-1 agonist is selected from native GLP-1, a GLP-1 analog, such as Liraglutide, Lixisenatide, Dulaglutide, Semaglutide, Exenatide, and a synthetic GLP-1.

10. The agonist combination for use according to any one of claims 3 to 7 wherein said GLP-1/GLP-2 dual agonist is a compound represented by the formula:
R¹—X*-U-R²
wherein:
R¹ is hydrogen (Hy), C₁₋₄ alkyl (e.g. methyl), acetyl, formyl, benzoyl or trifluoroacetyl;
R² is NH₂ or OH;
X* is a peptide of formula I:
(I)
H-X2-EG-X5-F-X7-X8-E-X10-X11-TIL-X15-X16-X17-A-
X19-X20-X21-FI-X24-WL-X27-X28-X29-KIT-X33
wherein:
X2 is Aib or G
X5 is T or S;
X7 is T or S;
X8 is S, E or D;
X10 is L, M, V or Ψ;
X11 is A, N or S;
X15 is D or E;
X16 is G, E, A or Ψ;
X17 is Q, E, K, L or Ψ;
X19 is A, V or S;
X20 is R, K or Ψ;
X21 is D, L or E;
X24 is A, N or S;
X27 is I, Q, K, H or Y;
X28 is Q, E, A, H, Y, L, K, R or S;
X29 is H, Y, K or Q;
X33 is D or E;
U is absent or a sequence of 1-15 residues each independently selected from K, k, E, A, T, I, L and Ψ;
the molecule contains one and only one Ψ, wherein Ψ is a residue of K, k, R, Orn, Dap or Dab in which the side chain is conjugated to a substituent having the formula Z¹— or Z¹—Z²—, wherein
Z¹— is CH₃—(CH₂)₁₀₋₂₂—(CO)— or HOOC—(CH₂)₁₀₋₂₂—(CO)—; and
—Z²— is selected from —Z^{S1}—, —Z^{S1}—Z^{S2}—, —Z^{S2}—Z^{S1}, —Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, —Z^{S2}Z^{S3}Z^{S2}— wherein
Z^{S1} is isoGlu, β-Ala, isoLys, or 4-aminobutanoyl;
Z^{S2} is -(Peg3)ₘ- where m is 1, 2, or 3; and
—Z^{S3}— is a peptide sequence of 1-6 amino acid units independently selected from the group consisting of A, L, S, T, Y, Q, D, E, K, k, R, H, F and G;
and wherein at least one of X5 and X7 is T;
or a pharmaceutically acceptable salt or solvate thereof.

11. The agonist combination for use according to any preceding claim wherein said agonist combination is in a composition, preferably a pharmaceutical composition.

12. The agonist combination for use according to any preceding claim wherein said agonist combination is administered at a dose of about 0.1 pmol/kg to 500 μmol/kg body weight, preferably about 50 pmol/kg to 500 nmol/kg.

13. The agonist combination for use according to any preceding claim, wherein said patient receives enteral nutrition within 48 hours of said surgical resection.

14. The agonist combination for use according to any preceding claim, wherein said patient has short bowel syndrome secondary to one or more of digestion disorders, malabsorption syndromes, short-gut syndrome, cul-de-sac syndrome, inflammatory bowel disease, celiac sprue (for example arising from gluten induced enteropathy or celiac disease), tropical sprue, hypogammaglobulinemic sprue, enteritis, ulcerative colitis, small intestine damage Crohn's disease, mesenteric infarction, volvulus, multiple strictures due to adhesions or radiation, vascular ischemia, necrotising enteral colitis (NEC), intestinal malformations, intestinal atresia, bowel cancer, surgical complications, acute injury, for example a stab injury.

15. The agonist combination for use according to any preceding claim, wherein the agonist combination is effective to increase villus growth, increase intestinal length, increase cell growth (trophic effect), increase weight gain, increase efficiency of nutrient uptake, increase mucosal height, and/or increase intestinal weight.


SEQUENCE LISTING (187 sequences)

SEQ ID NO: 1 - protein, 33 aa, Artificial Sequence
  GLP-1/GLP-2 dual agonist peptide
  MISC_FEATURE (2)..(2): Xaa is Aib or G
  MISC_FEATURE (5)..(5): Xaa is T or S
  MISC_FEATURE (7)..(7): Xaa is T or S
  MISC_FEATURE (8)..(8): Xaa is S, E or D
  MISC_FEATURE (10)..(10): Xaa is L, M, V or a residue of K, k (D-lys), R, Orn, Dap or Dab in which the side chain is conjugated to a substituent
  MISC_FEATURE (11)..(11): Xaa is A, N or S
  MISC_FEATURE (15)..(15): Xaa is D or E
  MISC_FEATURE (16)..(16): Xaa is G, E, A or a residue of K, k (D-lys), R, Orn, Dap or Dab in which the side chain is conjugated to a substituent
  MISC_FEATURE (17)..(17): Xaa is Q, E, K, L or a residue of K, k (D-lys), R, Orn, Dap or Dab in which the side chain is conjugated to a substituent
  MISC_FEATURE (19)..(19): Xaa is A, V or S
  MISC_FEATURE (20)..(20): Xaa is R, K or a residue of K, k (D-lys), R, Orn, Dap or Dab in which the side chain is conjugated to a substituent
  MISC_FEATURE (21)..(21): Xaa is D, L or E
  MISC_FEATURE (24)..(24): Xaa is A, N or  S
  MISC_FEATURE (27)..(27): Xaa is I, Q, K, H or Y
  MISC_FEATURE (28)..(28): Xaa is Q, E, A, H, Y, L, K, R or S
  MISC_FEATURE (29)..(29): Xaa is H, Y, K or Q
  MISC_FEATURE (33)..(33): Xaa is D or E
HXEGXFXXEXXTILXXXAXXXFIXWLXXXKITX

SEQ ID NO: 2 - protein, 33 aa, Artificial Sequence
  GLP-1/GLP-2 dual agonist peptide
  MISC_FEATURE (2)..(2): Xaa is Aib or G
  MISC_FEATURE (5)..(5): Xaa is T or S
  MISC_FEATURE (7)..(7): Xaa is T or S
  MISC_FEATURE (10)..(10): Xaa is L or a residue of K, k (D-lys), R, Orn, Dap or Dab in which the side chain is conjugated to a substituent
  MISC_FEATURE (11)..(11): Xaa is A or S
  MISC_FEATURE (15)..(15): Xaa is D or E
  MISC_FEATURE (16)..(16): Xaa is G, E, A or a residue of K, k (D-lys), R, Orn, Dap or Dab in which the side chain is conjugated to a substituent
  MISC_FEATURE (17)..(17): Xaa is Q, E, K, L or a residue of K, k (D-lys), R, Orn, Dap or Dab in which the side chain is conjugated to a substituent
  MISC_FEATURE (19)..(19): Xaa is A or S
  MISC_FEATURE (20)..(20): Xaa is R or a residue of K, k (D-lys), R, Orn, Dap or Dab in which the side chain is conjugated to a substituent
  MISC_FEATURE (21)..(21): Xaa is D, L or E
  MISC_FEATURE (27)..(27): Xaa is I, Q, K or Y
  MISC_FEATURE (28)..(28): Xaa is Q, E, A, H, Y, L, K, R or S
  MISC_FEATURE (29)..(29): Xaa is H, Y or Q
  MISC_FEATURE (33)..(33): Xaa is D or E
HXEGXFXSEXXTILXXXAXXXFIAWLXXXKITX

SEQ ID NO: 3 - protein, 33 aa, Artificial Sequence
  GLP-1/GLP-2 Dual agonist peptide
  MISC_FEATURE (2)..(2): Xaa is Aib or G
  MISC_FEATURE (5)..(5): Xaa is T or S
  MISC_FEATURE (7)..(7): Xaa is T or S
  MISC_FEATURE (16)..(16): Xaa is G or a residue of K, k (D-lys), R, Orn, Dap or Dab in which the side chain is conjugated to a substituent
  MISC_FEATURE (17)..(17): Xaa is Q, E, K, L or a residue of K, k (D-lys), R, Orn, Dap or Dab in which the side chain is conjugated to a substituent
  MISC_FEATURE (21)..(21): Xaa is D or L
  MISC_FEATURE (28)..(28): Xaa is Q, E, A, H, Y, L, K, R or S
  MISC_FEATURE (29)..(29): Xaa is H, Y or Q
HXEGXFXSELATILDXXAARXFIAWLIXXKITD

SEQ ID NO: 4 - protein, 33 aa, Artificial Sequence
  GLP-1/GLP-2 dual agonist peptide
  MOD_RES (2)..(2): Aib
  MISC_FEATURE (5)..(5): Xaa is T or S where at least one of Xaa at position 5 or 7 is T
  MISC_FEATURE (7)..(7): Xaa is T or S where at least one of Xaa at position 5 or 7 is T
  MISC_FEATURE (10)..(10): Xaa is L or a residue of K, k (D-lys), R, Orn, Dap or Dab in which the side chain is conjugated to a substituent
  MISC_FEATURE (16)..(16): Xaa is G, E, A or a residue of K, k (D-lys), R, Orn, Dap or Dab in which the side chain is conjugated to a substituent
  MISC_FEATURE (17)..(17): Xaa is Q, E, K, L or a residue of K, k (D-lys), R, Orn, Dap or Dab in which the side chain is conjugated to a substituent
  MISC_FEATURE (20)..(20): Xaa is R or a residue of K, k (D-lys), R, Orn, Dap or Dab in which the side chain is conjugated to a substituent
  MISC_FEATURE (21)..(21): Xaa is D or L
  MISC_FEATURE (28)..(28): Xaa is E, A or Q
  MISC_FEATURE (29)..(29): Xaa is H, Y or Q
HXEGXFXSEXATILDXXAAXXFIAWLIXXKITD

SEQ ID NO: 5 - protein, 33 aa, Artificial Sequence; identical to SEQ ID NO: 3
  GLP-1/GLP-2 Dual agonist peptide
  MOD_RES (2)..(2): Aib
  MISC_FEATURE (5)..(5): Xaa is T or S where at least one of Xaa at position 5 or 7 is T
  MISC_FEATURE (7)..(7): Xaa is T or S where at least one of Xaa at position 5 or 7 is T
  MISC_FEATURE (16)..(16): Xaa is G or a residue of K, k (D-lys), R, Orn, Dap or Dab in which the side chain is conjugated to a substituent
  MISC_FEATURE (17)..(17): Xaa is E, K, L or a residue of K, k (D-lys), R, Orn, Dap or Dab in which the side chain is conjugated to a substituent
  MISC_FEATURE (21)..(21): Xaa is D or L
  MISC_FEATURE (28)..(28): Xaa is E or A
  MISC_FEATURE (29)..(29): Xaa is H, Y or Q

SEQ ID NO: 6 - protein, 33 aa, Artificial Sequence
  GLP-1/GLP-2 Dual agonist peptide
  MOD_RES (2)..(2): Aib
  MISC_FEATURE (5)..(5): Xaa is T or S where at least one of Xaa at position 5 or 7 is T
  MISC_FEATURE (7)..(7): Xaa is T or S where at least one of Xaa at position 5 or 7 is T
  MISC_FEATURE (16)..(16): Xaa is a residue of K, k (D-lys), R, Orn, Dap or Dab in which the side chain is conjugated to a substituent
  MISC_FEATURE (28)..(28): Xaa is Q, E, A, H, Y, L, K, R or S, e.g. Q, E, A, H, Y or L
  MISC_FEATURE (29)..(29): Xaa is H, Y or Q
HXEGXFXSELATILDXQAARDFIAWLIXXKITD

SEQ ID NO: 7 - protein, 6 aa, Artificial Sequence
  GLP-1/GLP-2 Dual agonist peptide
  MISC_FEATURE (1)..(6): Lys at positions 1-6 is D-Lys
KKKKKK

SEQ ID NO: 8 - protein, 4 aa, Artificial Sequence
  GLP-1/GLP-2 Dual agonist peptide
  MOD_RES (1)..(1): Xaa is isoGlu
XKEK

SEQ ID NO: 9 - protein, 5 aa, Artificial Sequence
  GLP-1/GLP-2 Dual agonist peptide
  MISC_FEATURE (2)..(2): Xaa is [17-Carboxy-heptadecanoyl]-isoGlu
KXKEK

SEQ ID NO: 10 - protein, 5 aa, Artificial Sequence
  GLP-1/GLP-2 Dual agonist peptide
  MISC_FEATURE (2)..(2): Xaa is [19-Carboxy-nonadecanoyl]-isoGlu
KXKEK

SEQ ID NO: 11 - protein, 6 aa, Artificial Sequence
  GLP-1/GLP-2 Dual agonist peptide
EKEKEK

SEQ ID NO: 12 - protein, 6 aa, Artificial Sequence
  GLP-1/GLP-2 Dual agonist peptide
  MISC_FEATURE (2)..(2): Xaa is D-lys
  MISC_FEATURE (4)..(4): Xaa is D-lys
  MISC_FEATURE (6)..(6): Xaa is D-lys
EXEXEX

SEQ ID NO: 13 - protein, 6 aa, Artificial Sequence
  GLP-1/GLP-2 Dual agonist peptide
AKAAEK

SEQ ID NO: 14 - protein, 6 aa, Artificial Sequence
  GLP-1/GLP-2 Dual agonist peptide
AKEKEK

SEQ ID NO: 15 - protein, 6 aa, Artificial Sequence
  GLP-1/GLP-2 Dual agonist peptide
ATILEK

SEQ ID NO: 16 - protein, 6 aa, Artificial Sequence
  GLP-1/GLP-2 Dual agonist peptide
  MISC_FEATURE (6)..(6): Xaa is a residue of K, k (D-lys), R, Orn, Dap or Dab in which the side chain is conjugated to a substituent
EKEKEX

SEQ ID NO: 17 - protein, 6 aa, Artificial Sequence
  GLP-1/GLP-2 Dual agonist peptide
  MISC_FEATURE (2)..(2): Xaa is D-lys
  MISC_FEATURE (4)..(4): Xaa is D-lys
  MISC_FEATURE (6)..(6): Xaa is a residue of K, k (D-lys), R, Orn, Dap or Dab in which the side chain is conjugated to a substituent
EXEXEX

SEQ ID NO: 18 - protein, 6 aa, Artificial Sequence
  GLP-1/GLP-2 Dual agonist peptide
  MISC_FEATURE (6)..(6): Xaa is a residue of K, k (D-lys), R, Orn, Dap or Dab in which the side chain is conjugated to a substituent
AKAAEX

SEQ ID NO: 19 - protein, 6 aa, Artificial Sequence
  GLP-1/GLP-2 Dual agonist peptide
  MISC_FEATURE (6)..(6): Xaa is a residue of K, k (D-lys), R, Orn, Dap or Dab in which the side chain is conjugated to a substituent
AKEKEX

SEQ ID NO: 20 - protein, 6 aa, Artificial Sequence
  GLP-1/GLP-2 Dual agonist peptide
  MISC_FEATURE (6)..(6): xaa is a residue of K, k (D-lys), R, Orn, Dap or Dab in which the side chain is conjugated to a substituent
ATILEX

SEQ ID NO: 21 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXEAARDFIAWLIEHKITD

SEQ ID NO: 22 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXEAARDFIAWLIEHKITD

SEQ ID NO: 23 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXEAARDFIAWLIEHKITD

SEQ ID NO: 24 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXKAARDFIAWLIEHKITD

SEQ ID NO: 25 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXKAARDFIAWLIEHKITD

SEQ ID NO: 26 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXKAARDFIAWLIEHKITD

SEQ ID NO: 27 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGTFSSELATILDGXAARDFIAWLIEHKITD

SEQ ID NO: 28 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGSFTSELATILDGXAARDFIAWLIEHKITD

SEQ ID NO: 29 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGTFTSELATILDGXAARDFIAWLIEHKITD

SEQ ID NO: 30 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXLAARDFIAWLIEHKITD

SEQ ID NO: 31 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXLAARDFIAWLIEHKITD

SEQ ID NO: 32 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXLAARDFIAWLIEHKITD

SEQ ID NO: 33 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXLAARDFIAWLIAHKITD

SEQ ID NO: 34 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXLAARDFIAWLIAHKITD

SEQ ID NO: 35 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXLAARDFIAWLIAHKITD

SEQ ID NO: 36 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXEAARLFIAWLIEHKITD

SEQ ID NO: 37 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXQAARDFIAWLIQHKITD

SEQ ID NO: 38 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIQHKITD

SEQ ID NO: 39 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXQAARDFIAWLIQHKITD

SEQ ID NO: 40 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXQAARDFIAWLIEHKITD

SEQ ID NO: 41 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXQAARDFIAWLIAHKITD

SEQ ID NO: 42 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIAHKITD

SEQ ID NO: 43 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXQAARDFIAWLIAHKITD

SEQ ID NO: 44 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIEHKITD

SEQ ID NO: 45 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXQAARDFIAWLIEHKITD

SEQ ID NO: 46 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIHHKITD

SEQ ID NO: 47 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIYHKITD

SEQ ID NO: 48 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLILHKITD

SEQ ID NO: 49 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIKHKITD

SEQ ID NO: 50 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIRHKITD

SEQ ID NO: 51 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLISHKITD

SEQ ID NO: 52 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLQQHKITD

SEQ ID NO: 53 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLYQHKITD

SEQ ID NO: 54 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLKQHKITD

SEQ ID NO: 55 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIQQKITD

SEQ ID NO: 56 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIQYKITD

SEQ ID NO: 57 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELSTILEXQASREFIAWLIAYKITE

SEQ ID NO: 58 - protein, 39 aa, Artificial Sequence
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa is a residue of K, k (D-lys), R, Orn, Dap or Dab in which the side chain is conjugated to a substituent
HXEGTFSSELATILDEQAARDFIAWLIAHKITDXXXXXX

SEQ ID NO: 59 - protein, 39 aa, Artificial Sequence
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa is a residue of K, k (D-lys), R, Orn, Dap or Dab in which the side chain is conjugated to a substituent
HXEGTFTSELATILDEQAARDFIAWLIAHKITDXXXXXX

SEQ ID NO: 60 - protein, 39 aa, Artificial Sequence
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa is a residue of K, k (D-lys), R, Orn, Dap or Dab in which the side chain is conjugated to a substituent
HXEGSFTSELATILDEQAARDFIAWLIEHKITDXXXXXX

SEQ ID NO: 61 - protein, 33 aa, Artificial Sequence
  GLP-1/GLP-2 Dual agonist peptide
  MOD_RES (2)..(2): Aib
  MISC_FEATURE (10)..(10): Xaa is a residue of K, k (D-lys), R, Orn, Dap or Dab in which the side chain is conjugated to a substituent
HXEGSFTSEXATILDEQAARDFIAWLIEHKITD

SEQ ID NO: 62 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGSFTSELATILEGXAARDFIAWLIEHKITD

SEQ ID NO: 63 - protein, 33 aa, Artificial Sequence
  GLP-1/GLP-2 Dual agonist peptide
  MOD_RES (2)..(2): Aib
  MISC_FEATURE (20)..(20): Xaa is a residue of K, k (D-lys), R, Orn, Dap or Dab in which the side chain is conjugated to a substituent
HXEGSFTSELATILDEQAAXDFIAWLIEHKITD

SEQ ID NO: 64 - protein, 33 aa, Artificial Sequence
  GLP-1/GLP-2 Dual agonist peptide
  MOD_RES (2)..(2): Aib
  MISC_FEATURE (20)..(20): Xaa is a residue of K, k (D-lys), R, Orn, Dap or Dab in which the side chain is conjugated to a substituent
HXEGTFTSELATILDEQAAXDFIAWLIEHKITD

SEQ ID NO: 65 - protein, 33 aa, Artificial Sequence
  GLP-1/GLP-2 Dual agonist peptide
  MOD_RES (2)..(2): Aib
  MISC_FEATURE (10)..(10): Xaa is a residue of K, k (D-lys), R, Orn, Dap or Dab in which the side chain is conjugated to a substituent
HXEGTFTSEXATILDEQAARDFIAWLIEHKITD

SEQ ID NO: 66 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGSFTSELATILDAXAARDFIAWLIEHKITD

SEQ ID NO: 67 - protein, 33 aa, Artificial Sequence
  GLP-1/GLP-2 Dual agonist peptide
  MOD_RES (2)..(2): Aib
  MISC_FEATURE (20)..(20): Xaa is a residue of K, k (D-lys), R, Orn, Dap or Dab in which the side chain is conjugated to a substituent
HXEGSFTSELATILDAKAAXDFIAWLIEHKITD

SEQ ID NO: 68 - protein, 33 aa, Artificial Sequence; identical to SEQ ID NO: 21
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 69 - protein, 33 aa, Artificial Sequence; identical to SEQ ID NO: 22
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 70 - protein, 33 aa, Artificial Sequence; identical to SEQ ID NO: 23
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 71 - protein, 33 aa, Artificial Sequence; identical to SEQ ID NO: 24
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 72 - protein, 33 aa, Artificial Sequence; identical to SEQ ID NO: 25
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 73 - protein, 33 aa, Artificial Sequence; identical to SEQ ID NO: 26
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 74 - protein, 33 aa, Artificial Sequence; identical to SEQ ID NO: 27
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 75 - protein, 33 aa, Artificial Sequence; identical to SEQ ID NO: 28
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 76 - protein, 33 aa, Artificial Sequence; identical to SEQ ID NO: 29
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 77 - protein, 33 aa, Artificial Sequence; identical to SEQ ID NO: 30
  GLP-1/GLP-2 Dual agonist peptide
  MOD_RES (2)..(2): Aib
  misc_feature (16)..(16): Xaa can be any naturally occurring amino acid
  MISC_FEATURE (17)..(17): Xaa indicates an L-lysine residue in which the side chain is conjugated to the substituent Z1- or Z1Z2

SEQ ID NO: 78 - protein, 33 aa, Artificial Sequence; identical to SEQ ID NO: 31
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 79 - protein, 33 aa, Artificial Sequence; identical to SEQ ID NO: 32
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 80 - protein, 33 aa, Artificial Sequence; identical to SEQ ID NO: 33
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 81 - protein, 33 aa, Artificial Sequence; identical to SEQ ID NO: 34
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 82 - protein, 33 aa, Artificial Sequence; identical to SEQ ID NO: 35
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 83 - protein, 33 aa, Artificial Sequence; identical to SEQ ID NO: 36
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 84 - protein, 33 aa, Artificial Sequence; identical to SEQ ID NO: 37
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 85 - protein, 33 aa, Artificial Sequence; identical to SEQ ID NO: 38
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 86 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 87 - protein, 33 aa, Artificial Sequence; identical to SEQ ID NO: 40
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 88 - protein, 33 aa, Artificial Sequence; identical to SEQ ID NO: 41
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 89 - protein, 33 aa, Artificial Sequence; identical to SEQ ID NO: 42
  GLP-1/GLP-2 Dual agonist peptide
  MOD_RES (2)..(2): Aib
  MISC_FEATURE (16)..(16): Xaa indicates an L-lysine residue respectively in which the side chain is conjugated to the substituent Z1- or Z1Z2
  MISC_FEATURE (16)..(16): Xaa indicates an L-lysine residue in which the side chain is conjugated to the substituent Z1- or Z1Z2

SEQ ID NO: 90 - protein, 33 aa, Artificial Sequence; identical to SEQ ID NO: 43
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 91 - protein, 33 aa, Artificial Sequence; identical to SEQ ID NO: 44
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 92 - protein, 33 aa, Artificial Sequence; identical to SEQ ID NO: 45
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 93 - protein, 33 aa, Artificial Sequence; identical to SEQ ID NO: 46
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 94 - protein, 33 aa, Artificial Sequence; identical to SEQ ID NO: 47
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 95 - protein, 33 aa, Artificial Sequence; identical to SEQ ID NO: 48
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 96 - protein, 33 aa, Artificial Sequence; identical to SEQ ID NO: 49
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 97 - protein, 33 aa, Artificial Sequence; identical to SEQ ID NO: 50
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 98 - protein, 33 aa, Artificial Sequence; identical to SEQ ID NO: 51
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 99 - protein, 33 aa, Artificial Sequence; identical to SEQ ID NO: 52
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 100 - protein, 33 aa, Artificial Sequence; identical to SEQ ID NO: 53
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 101 - protein, 33 aa, Artificial Sequence; identical to SEQ ID NO: 54
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 102 - protein, 33 aa, Artificial Sequence; identical to SEQ ID NO: 55
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 103 - protein, 33 aa, Artificial Sequence; identical to SEQ ID NO: 56
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 104 - protein, 33 aa, Artificial Sequence; identical to SEQ ID NO: 57
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 105 - protein, 39 aa, Artificial Sequence; identical to SEQ ID NO: 58
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa indicates an D-lysine residue in which the side chain is conjugated to the substituent Z1- or Z1Z2

SEQ ID NO: 106 - protein, 39 aa, Artificial Sequence; identical to SEQ ID NO: 59
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa indicates an D-lysine residue in which the side chain is conjugated to the substituent Z1- or Z1Z2

SEQ ID NO: 107 - protein, 39 aa, Artificial Sequence; identical to SEQ ID NO: 60
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa indicates an D-lysine residue in which the side chain is conjugated to the substituent Z1- or Z1Z2

SEQ ID NO: 108 - protein, 33 aa, Artificial Sequence; identical to SEQ ID NO: 61
  GLP-1/GLP-2 Dual agonist peptide
  MOD_RES (2)..(2): Aib
  MISC_FEATURE (10)..(10): Xaa indicates an L-lysine residue in which the side chain is conjugated to the substituent Z1- or Z1Z2

SEQ ID NO: 109 - protein, 33 aa, Artificial Sequence; identical to SEQ ID NO: 62
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 110 - protein, 33 aa, Artificial Sequence; identical to SEQ ID NO: 63
  GLP-1/GLP-2 Dual agonist peptide
  MOD_RES (2)..(2): Aib
  MISC_FEATURE (20)..(20): Xaa indicates an L-lysine residue in which the side chain is conjugated to the substituent Z1- or Z1Z2

SEQ ID NO: 111 - protein, 33 aa, Artificial Sequence; identical to SEQ ID NO: 64
  GLP-1/GLP-2 Dual agonist peptide
  MOD_RES (2)..(2): Aib
  MISC_FEATURE (20)..(20): Xaa indicates an L-lysine residue in which the side chain is conjugated to the substituent Z1- or Z1Z2

SEQ ID NO: 112 - protein, 33 aa, Artificial Sequence; identical to SEQ ID NO: 65
  GLP-1/GLP-2 Dual agonist peptide
  MOD_RES (2)..(2): Aib
  MISC_FEATURE (10)..(10): Xaa indicates an L-lysine residue in which the side chain is conjugated to the substituent Z1- or Z1Z2

SEQ ID NO: 113 - protein, 33 aa, Artificial Sequence; identical to SEQ ID NO: 66
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 114 - protein, 33 aa, Artificial Sequence; identical to SEQ ID NO: 67
  GLP-1/GLP-2 Dual agonist peptide
  MOD_RES (2)..(2): Aib
  MISC_FEATURE (20)..(20): Xaa indicates an L-lysine residue in which the side chain is conjugated to the substituent Z1- or Z1Z2

SEQ ID NO: 115 - protein, 33 aa, Artificial Sequence; identical to SEQ ID NO: 21
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 116 - protein, 33 aa, Artificial Sequence; identical to SEQ ID NO: 22
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 117 - protein, 33 aa, Artificial Sequence; identical to SEQ ID NO: 23
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 118 - protein, 33 aa, Artificial Sequence; identical to SEQ ID NO: 24
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 119 - protein, 33 aa, Artificial Sequence; identical to SEQ ID NO: 25
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 120 - protein, 33 aa, Artificial Sequence; identical to SEQ ID NO: 26
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 121 - protein, 33 aa, Artificial Sequence; identical to SEQ ID NO: 27
  GLP-1/GLP-2 Dual agonist peptide
  MOD_RES (2)..(2): Aib
  MISC_FEATURE (17)..(17): Xaa is K([17-carboxy-heptadecanoyl]-isoGlu)

SEQ ID NO: 122 - protein, 33 aa, Artificial Sequence; identical to SEQ ID NO: 28
  GLP-1/GLP-2 Dual agonist peptide
  MOD_RES (2)..(2): Aib
  MISC_FEATURE (17)..(17): Xaa is K([17-carboxy-heptadecanoyl]-isoGlu)

SEQ ID NO: 123 - protein, 33 aa, Artificial Sequence; identical to SEQ ID NO: 29
  GLP-1/GLP-2 Dual agonist peptide
  MOD_RES (2)..(2): Aib
  MISC_FEATURE (17)..(17): Xaa  is K([17-carboxy-heptadecanoyl]-isoGlu)

SEQ ID NO: 124 - protein, 33 aa, Artificial Sequence; identical to SEQ ID NO: 30
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 125 - protein, 33 aa, Artificial Sequence; identical to SEQ ID NO: 31
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 126 - protein, 33 aa, Artificial Sequence; identical to SEQ ID NO: 32
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 127 - protein, 33 aa, Artificial Sequence; identical to SEQ ID NO: 33
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 128 - protein, 33 aa, Artificial Sequence; identical to SEQ ID NO: 34
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 129 - protein, 33 aa, Artificial Sequence; identical to SEQ ID NO: 35
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 130 - protein, 33 aa, Artificial Sequence; identical to SEQ ID NO: 36
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 131 - protein, 33 aa, Artificial Sequence; identical to SEQ ID NO: 37
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 132 - protein, 33 aa, Artificial Sequence; identical to SEQ ID NO: 38
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 133 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 134 - protein, 33 aa, Artificial Sequence; identical to SEQ ID NO: 40
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 135 - protein, 33 aa, Artificial Sequence; identical to SEQ ID NO: 41
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 136 - protein, 33 aa, Artificial Sequence; identical to SEQ ID NO: 42
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 137 - protein, 33 aa, Artificial Sequence; identical to SEQ ID NO: 43
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 138 - protein, 33 aa, Artificial Sequence; identical to SEQ ID NO: 44
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 139 - protein, 33 aa, Artificial Sequence; identical to SEQ ID NO: 45
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 140 - protein, 33 aa, Artificial Sequence; identical to SEQ ID NO: 46
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 141 - protein, 33 aa, Artificial Sequence; identical to SEQ ID NO: 47
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 142 - protein, 33 aa, Artificial Sequence; identical to SEQ ID NO: 48
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 143 - protein, 33 aa, Artificial Sequence; identical to SEQ ID NO: 49
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 144 - protein, 33 aa, Artificial Sequence; identical to SEQ ID NO: 50
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 145 - protein, 33 aa, Artificial Sequence; identical to SEQ ID NO: 51
  GLP-1/GLP-2 Dual agonist peptide
  MOD_RES (2)..(2): Aib
  misc (16)..(16): Xaa is K([17-carboxy-heptadecanoyl]-isoGlu)

SEQ ID NO: 146 - protein, 33 aa, Artificial Sequence; identical to SEQ ID NO: 52
  GLP-1/GLP-2 Dual agonist peptide
  MOD_RES (2)..(2): Aib
  MISC_FEATURE (16)..(16): Xaa is K([Hexadecanoyl]- betaAla)

SEQ ID NO: 147 - protein, 33 aa, Artificial Sequence; identical to SEQ ID NO: 53
  GLP-1/GLP-2 Dual agonist peptide
  MOD_RES (2)..(2): Aib
  MISC_FEATURE (16)..(16): Xaa is K([17-carboxy-heptadecanoyl]iso-Glu- Peg3)

SEQ ID NO: 148 - protein, 33 aa, Artificial Sequence; identical to SEQ ID NO: 54
  GLP-1/GLP-2 Dual agonist peptide
  MOD_RES (2)..(2): Aib
  MISC_FEATURE (16)..(16): Xaa is K([19-carboxy-nonadecanoyl]iso-Glu-Peg3- Peg3)

SEQ ID NO: 149 - protein, 33 aa, Artificial Sequence; identical to SEQ ID NO: 55
  GLP-1/GLP-2 Dual agonist peptide
  MOD_RES (2)..(2): Aib
  MISC_FEATURE (16)..(16): Xaa is K([19-carboxy-nonadecanoyl]iso-Lys-Peg3- Peg3-Peg3)

SEQ ID NO: 150 - protein, 33 aa, Artificial Sequence; identical to SEQ ID NO: 56
  GLP-1/GLP-2 Dual agonist peptide
  MOD_RES (2)..(2): Aib
  MISC_FEATURE (16)..(16): Xaa is K(Octadecanoyl)

SEQ ID NO: 151 - protein, 33 aa, Artificial Sequence; identical to SEQ ID NO: 57
  GLP-1/GLP-2 Dual agonist peptide
  MOD_RES (2)..(2): Aib
  MISC_FEATURE (16)..(16): Xaa is K(Hexadecanoyl-isoGlu)

SEQ ID NO: 152 - protein, 40 aa, Artificial Sequence
  GLP-1/GLP-2 Dual agonist peptide
  MOD_RES (2)..(2): Aib
  MISC_FEATURE (34)..(39): Xaa at positions 34-39 is D-Lys
  MISC_FEATURE (40)..(40): Xaa is ([17-carboxy-Heptadecanoyl]-isoGlu)
HXEGTFSSELATILDEQAARDFIAWLIAHKITDXXXXXXX

SEQ ID NO: 153 - protein, 40 aa, Artificial Sequence
  GLP-1/GLP-2 Dual agonist peptide
  MOD_RES (2)..(2): Aib
  MISC_FEATURE (34)..(39): Xaa at positions 34-39 is D-Lys
  MISC_FEATURE (40)..(40): Xaa is ([17-carboxy-Heptadecanoyl]-isoGlu)
HXEGTFTSELATILDEQAARDFIAWLIAHKITDXXXXXXX

SEQ ID NO: 154 - protein, 40 aa, Artificial Sequence
  GLP-1/GLP-2 Dual agonist peptide
  MOD_RES (2)..(2): Aib
  MISC_FEATURE (34)..(39): Xaa at positions 34-39 is D-Lys
  MISC_FEATURE (40)..(40): Xaa is ([17-carboxy-Heptadecanoyl]-isoGlu)
HXEGSFTSELATILDEQAARDFIAWLIEHKITDXXXXXXX

SEQ ID NO: 155 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa is K([19-Carboxy-nonadecanoyl]-isoGlu)

SEQ ID NO: 156 - protein, 33 aa, Artificial Sequence; identical to SEQ ID NO: 61
  GLP-1/GLP-2 Dual agonist peptide
  MOD_RES (2)..(2): Aib
  MISC_FEATURE (10)..(10): Xaa is K([19-carboxy-nonadecanoyl]iso-Glu-Peg3- Peg3)

SEQ ID NO: 157 - protein, 33 aa, Artificial Sequence; identical to SEQ ID NO: 25
  GLP-1/GLP-2 Dual agonist peptide
  MOD_RES (2)..(2): Aib
  MISC_FEATURE (16)..(16): Xaa is K([19-carboxy-nonadecanoyl]iso-Glu-Peg3- Peg3)

SEQ ID NO: 158 - protein, 33 aa, Artificial Sequence; identical to SEQ ID NO: 62
  GLP-1/GLP-2 Dual agonist peptide
  MOD_RES (2)..(2): Aib
  MISC_FEATURE (17)..(17): Xaa is K([19-carboxy-nonadecanoyl]iso-Glu-Peg3- Peg3)

SEQ ID NO: 159 - protein, 33 aa, Artificial Sequence; identical to SEQ ID NO: 63
  GLP-1/GLP-2 Dual agonist peptide
  MOD_RES (2)..(2): Aib
  MISC_FEATURE (20)..(20): Xaa is K([19-carboxy-nonadecanoyl]iso-Glu-Peg3- Peg3)

SEQ ID NO: 160 - protein, 33 aa, Artificial Sequence; identical to SEQ ID NO: 64
  GLP-1/GLP-2 Dual agonist peptide
  MOD_RES (2)..(2): Aib
  MISC_FEATURE (20)..(20): Xaa is K([19-carboxy-nonadecanoyl]iso-Glu-Peg3- Peg3)

SEQ ID NO: 161 - protein, 33 aa, Artificial Sequence; identical to SEQ ID NO: 37
  GLP-1/GLP-2 Dual agonist peptide
  MOD_RES (2)..(2): Aib
  MISC_FEATURE (16)..(16): Xaa is K([17-Carboxy-heptadecanoyl]-isoGlu-KEK- Peg3)

SEQ ID NO: 162 - protein, 33 aa, Artificial Sequence; identical to SEQ ID NO: 37
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 163 - protein, 33 aa, Artificial Sequence; identical to SEQ ID NO: 40
  GLP-1/GLP-2 Dual agonist peptide
  MOD_RES (2)..(2): Aib
  MISC_FEATURE (16)..(16): Xaa is K([17-Carboxy-heptadecanoyl]-isoGlu-KEK- Peg3)

SEQ ID NO: 164 - protein, 33 aa, Artificial Sequence; identical to SEQ ID NO: 40
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 165 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK)

SEQ ID NO: 166 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 167 - protein, 33 aa, Artificial Sequence; identical to SEQ ID NO: 61
  GLP-1/GLP-2 Dual agonist peptide
  MOD_RES (2)..(2): Aib
  MISC_FEATURE (10)..(10): Xaa is K([19-Carboxy-nonadecanoyl]-isoGlu-KEK- Peg3)

SEQ ID NO: 168 - protein, 33 aa, Artificial Sequence; identical to SEQ ID NO: 65
  GLP-1/GLP-2 Dual agonist peptide
  MOD_RES (2)..(2): Aib
  MISC_FEATURE (10)..(10): Xaa is K([19-Carboxy-nonadecanoyl]-isoGlu-KEK- Peg3)

SEQ ID NO: 169 - protein, 33 aa, Artificial Sequence; identical to SEQ ID NO: 61
  GLP-1/GLP-2 Dual agonist peptide
  MOD_RES (2)..(2): Aib
  MISC_FEATURE (10)..(10): Xaa is K([19-carboxy-nonadecanoyl]iso-Glu-KEK- Peg3-Peg3)

SEQ ID NO: 170 - protein, 33 aa, Artificial Sequence; identical to SEQ ID NO: 45
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 171 - protein, 33 aa, Artificial Sequence; identical to SEQ ID NO: 44
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 172 - protein, 33 aa, Artificial Sequence; identical to SEQ ID NO: 42
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 173 - protein, 33 aa, Artificial Sequence; identical to SEQ ID NO: 25
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 174 - protein, 33 aa, Artificial Sequence; identical to SEQ ID NO: 44
  GLP-1/GLP-2 Dual agonist peptide
  MOD_RES (2)..(2): Aib
  MISC_FEATURE (16)..(16): Xaa is K([19-carboxy-nonadecanoyl]iso-Glu-KEK- Peg3-Peg3)

SEQ ID NO: 175 - protein, 33 aa, Artificial Sequence; identical to SEQ ID NO: 62
  GLP-1/GLP-2 Dual agonist peptide
  MOD_RES (2)..(2): Aib
  MISC_FEATURE (17)..(17): Xaa is K([19-Carboxy-nonadecanoyl]-isoGlu-KEK- Peg3)

SEQ ID NO: 176 - protein, 33 aa, Artificial Sequence; identical to SEQ ID NO: 66
  GLP-1/GLP-2 Dual agonist peptide
  MOD_RES (2)..(2): Aib
  MISC_FEATURE (17)..(17): Xaa is K([19-Carboxy-nonadecanoyl]-isoGlu-KEK- Peg3)

SEQ ID NO: 177 - protein, 33 aa, Artificial Sequence; identical to SEQ ID NO: 66
  GLP-1/GLP-2 Dual agonist peptide
  MOD_RES (2)..(2): Aib
  MISC_FEATURE (17)..(17): Xaa is K([19-carboxy-nonadecanoyl]iso-Glu-KEK- Peg3-Peg3)

SEQ ID NO: 178 - protein, 33 aa, Artificial Sequence; identical to SEQ ID NO: 63
  GLP-1/GLP-2 Dual agonist peptide
  MOD_RES (2)..(2): Aib
  MISC_FEATURE (20)..(20): Xaa is K([19-Carboxy-nonadecanoyl]-isoGlu-KEK- Peg3)

SEQ ID NO: 179 - protein, 33 aa, Artificial Sequence; identical to SEQ ID NO: 64
  GLP-1/GLP-2 Dual agonist peptide
  MOD_RES (2)..(2): Aib
  MISC_FEATURE (20)..(20): Xaa is K([19-Carboxy-nonadecanoyl]-isoGlu-KEK- Peg3)

SEQ ID NO: 180 - protein, 33 aa, Artificial Sequence; identical to SEQ ID NO: 63
  GLP-1/GLP-2 Dual agonist peptide
  MOD_RES (2)..(2): Aib
  MISC_FEATURE (20)..(20): Xaa is K([19-carboxy-nonadecanoyl]iso-Glu-KEK- Peg3-Peg3)

SEQ ID NO: 181 - protein, 33 aa, Artificial Sequence; identical to SEQ ID NO: 64
  GLP-1/GLP-2 Dual agonist peptide
  MOD_RES (2)..(2): Aib
  MISC_FEATURE (20)..(20): Xaa is K([19-carboxy-nonadecanoyl]iso-Glu-KEK- Peg3-Peg3)

SEQ ID NO: 182 - protein, 33 aa, Artificial Sequence; identical to SEQ ID NO: 67
  GLP-1/GLP-2 Dual agonist peptide
  MOD_RES (2)..(2): Aib
  MISC_FEATURE (20)..(20): Xaa is K([19-Carboxy-nonadecanoyl]-isoGlu-KEK- Peg3)

SEQ ID NO: 183 - protein, 4 aa, Artificial Sequence
  GLP-1/GLP-2 Dual agonist peptide
  MISC_FEATURE (1)..(1): Xaa is betaAla
XKEK

SEQ ID NO: 184 - protein, 4 aa, Artificial Sequence
  GLP-1/GLP-2 Dual agonist peptide
  MISC_FEATURE (1)..(1): Xaa is isoLys
XKEK

SEQ ID NO: 185 - protein, 4 aa, Artificial Sequence
  GLP-1/GLP-2 Dual agonist peptide
  MISC_FEATURE (4)..(4): Xaa is isoGlu
KEKX

SEQ ID NO: 186 - protein, 4 aa, Artificial Sequence
  GLP-1/GLP-2 Dual agonist peptide
  MISC_FEATURE (4)..(4): Xaa is betaAla
KEKX

SEQ ID NO: 187 - protein, 4 aa, Artificial Sequence
  GLP-1/GLP-2 Dual agonist peptide
  MISC_FEATURE (4)..(4): Xaa is isoLys
KEKX